(19) United States
(12) Patent Application Publication
(10) Pub. No.: US 2020/0080081 A1
(43) Pub. Date: Mar. 12, 2020

(54) A METHOD FOR REGULATING THE FUNCTION OF A HEART CELL, RELATED NUCLEOTIDES AND COMPOUNDS

(71) Applicant: Agency for Science, Technology and Research, Singapore (SG)

(72) Inventors: Kelvin Zhenwei SEE, Singapore (SG); Roger Sik Yin FOO, Singapore (SG)

(73) Assignee: Agency for Science, Technology and Research, Singapore (SG)

(21) Appl. No.: 16/469,603

(22) PCT Filed: Dec. 14, 2017
(86) PCT No.: PCT/SG2017/050620

§ 371 Date: Jun. 13, 2019

(30) Foreign Application Priority Data
Dec. 14, 2016 (SG) 10201610502Y

(51) Int. Cl. C12N 15/113; C12Q 1/6881; C12N 15/115; A01K 67/027; A61P 9/00

(52) U.S. Cl. CPC C12N 15/113; C12N 2999/007; C12N 15/115; A01K 67/0276; A61P 9/00; A01K 2207/30; A01K 2217/052; A01K 2227/105; A01K 2267/0375; C12N 2310/113; C12N 2310/20; C12N 2750/14143; C12Q 2600/118; C12Q 2600/158; C12Q 2600/178; C12N 2310/14; C12Q 1/6881

(57) ABSTRACT

The invention relates to inhibitors of Sghrt and/or Gas5 lincRNAs, in particular to polynucleotides complementary to coding and non-coding sequences of said lincRNAs, and methods of producing said inhibitors. Also disclosed are the use of the afore agents to proliferate, regenerate or dedifferentiate a heart cell; methods for preventing and treating cardiac disease using the afore agents; and a prognostic or diagnostic assay to assess the regenerative or proliferative capacity of heart tissue before, after or during a cardiac treatment regimen, comprising determining the presence or amount of Sghrt and/or Gas5 lincRNAs. The present disclosure also relates to a method for screening for a therapeutic agent that can be used to treat or prevent a heart disorder, comprising analysing the functional expression and/or expression level of Sghrt and/or Gas5 lincRNAs in the presence and in the absence of the therapeutic agent.

### FIELD OF THE INVENTION

The invention relates to inhibitors of genes or lincRNAs in cardiomyocytes (LINCMs), in particular to polynucleotides having the ability to stimulate cardiac regeneration or proliferation and their use as cardio protective and/or cardio regenerative agents; methods for preventing and treating cardiac disease using the afore agents; the use of the afore agents to prevent or treat cardiac disease; and a prognostic or diagnostic assay to assess the regenerative or proliferative capacity of heart tissue before after or during a cardiac treatment regimen.

### BACKGROUND OF THE INVENTION

In the lifetime of an adult mouse or human heart, new cardiomyocytes (CMs) are generated albeit at very low rates of ˜1%. On the other hand, adult zebrafish and neonatal mouse hearts can fully regenerate upon surgical resection or infarct injury. Like the zebrafish and neonatal mouse, new CMs in the adult mouse appear to arise by mitosis of pre-existing CMs, but a sufficient level of endogenous mitosis is lacking to allow for adequate regeneration and repair during disease progression. Loss of the full capacity to regenerate occurs soon after the seventh postnatal day (P7) when CMs in the neonatal mouse heart exit the cell cycle.

This highlights two key questions for the field of cardiac regeneration: a) what holds back adult CMs from dividing and b) can any adult CM be induced to divide? Indeed lineage tracing studies in regenerating hearts of zebrafish and neonatal mice, show that proliferation potency is achieved by cell cycle re-entry of pre-existing CMs. Consistent with this, Hippo/Yap pathway components, the transcription factor Meis1, and a series of microRNA including members of the miR-15 family, miR-199a, miR-590, miR-17-92 cluster, miR-99/10, and Let-7a/c have been separately implicated in the regulation of CM proliferation. Others have shown that while the majority of CMs in adult mouse hearts permanently exit the cell cycle, a rare subset existing in relatively hypoxic microenvironment of the myocardium, retain proliferative neonatal CM features, and have smaller size, mono-nucleation and lower oxidative DNA damage. Although this specialized subset of CM may explain the ˜1% endogenous proliferation capacity in the adult myocardium, it remains unexplored whether heterogeneity of the stress-response gene expression changes among the larger majority of cell cycle-arrested CMs would uncover a sub-population that could be motivated to re-enter cell cycle.

Adult mammalian cardiomyocytes (CMs) rarely proliferate and this low rate of mitosis in pre-existing CMs and low percentage of endogenous cardiac progenitor cells in adult hearts preclude effective regeneration of new CMs during pathological conditions such as myocardial infarction or heart failure.

It is still unclear how the proliferation window is controlled in post-natal CMs and whether adult mouse CMs require a dedifferentiation step prior to re-entering the cell cycle, or indeed if they can be induced to re-enter cell cycle directly without dedifferentiation during the pathological disease stress response. The pathophysiology of both myocardial infarction and heart failure critically involves CM cell loss, and in both cases, cardiac regeneration would make for a novel therapy that could reverse the course of each disease.

Despite the complexity of CM proliferation, serendipitously, we have identified two novel endogenous regulators of CM proliferation. With this knowledge we have devised inhibitors that can regulate CM proliferation in a favourable manner and so encourage cardiac repair.

Indeed, we have mapped out gene regulatory networks that contained key nodal lincRNAs (Gas5 and Sghrt) that regulate dedifferentiation and cell cycle gene expression in CM subpopulations. We have investigated the functional role of these lincRNAs and shown that Gas5 and Sghrt regulate dedifferentiation and cell cycle checkpoints in vitro and in vivo. Moreover, Gas5 and Sghrt are able to extend the proliferation window of neonatal CMs to beyond P7. Our work has thus identified the first lincRNAs to negatively regulate the cell cycle in post-natal CMs and so inhibited cell proliferation.

### STATEMENTS OF INVENTION

According to a first aspect of the invention there is provided at least one inhibitor for inhibiting any one or more of the following:
a) a Sghrt lincRNA and/or a Gas5 lincRNA;
b) one or both of Sghrt gene and/or Gas5 gene transcription to produce a Sghrt lincRNA and Gas 5 lincRNA;
   comprising an isolated polynucleotide comprising or consisting of a sequence:
i) that is complementary to any one of Sghrt lincRNAs and/or any one of Gas5 lincRNAs or their coding sequences i.e. SEQ ID NO:1-54 or a part thereof; or
ii) that is complementary to any one of gDNA Sghrt non-coding sequences provided in SEQ ID NO:67, or a part thereof and/or gDNA Gas5 non-coding sequences provided in SEQ ID NO:68, or a part thereof; or
iii) a sequence that shares at least 75% identity with the polynucleotide of i) and/or ii).

Reference herein to an inhibitor, is to a polynucleotide that is capable of interacting with said lincRNAs of Sghrt and/or Gas5 in a manner that prevents their function or to a polynucleotide that is capable of interacting with said Sghrt gene and/or Gas5gene in a manner that prevents their transcription to produce lincRNAs Sghrt and Gas5. In this way, the inhibitor is able to overcome the negative regulatory role of said lincRNAs and so encourage or support division, proliferation, regeneration and/or dedifferentiation of a heart cell.

Thus, the invention concerns the realisation that lincRNAs of Sghrt and Gas5 have an inhibitory effect on heart tissue proliferation or regeneration and thus their inhibition, or the removal of their negative influence, can be used to encourage, support or provide for heart tissue proliferation or regeneration.

In a preferred embodiment of the invention said isolated and complementary polynucleotide interacts with its complementary sequence to block the function of same.

Most preferably said isolated and complementary polynucleotide interacts with said lincRNAs of Sghrt and/or Gas5 and so is complementary to any one of SEQ ID NOs:1, 3, 5, 7, 9, 11, 13, 15, 17, 19, 21, 23, 25, 27, 29, 31, 33, 35, 37, 39, 41, 43, 45, 47, 49, 51-53, or a part thereof. More particularly, said isolated and complementary polynucleotide interacts with said lincRNAs of Sghrt and so is complementary to any one of SEQ ID NOs:51-53, or a part thereof. Alternatively, said isolated and complementary polynucleotide interacts with said lincRNAs of Gas5 and so is complementary to any one of SEQ ID NOs:1, 3, 5, 7, 9, 11, 13, 15, 17, 19, 21, 23, 25, 27, 29, 31, 33, 35, 37, 39, 41, 43, 45, 47 and 49 or a part thereof.

Most preferably still said isolated and complementary polynucleotide interacts with said coding region for said lincRNAs of Sghrt and so is complementary to SEQ ID NOs:54, or a part thereof. More particularly, said isolated and complementary polynucleotide interacts with said coding region for said lincRNAs of Gas5 and so is complementary to any one of SEQ ID NOs:2, 4, 6, 8, 10, 12, 14, 16, 18, 20, 22, 24, 26, 28, 30, 32, 34, 36, 38, 40, 42, 44, 46, 48 and 50, or a part thereof.

Alternatively, said isolated and complementary polynucleotide interacts with said Sghrt and/or Gas5 gene and so is complementary to any one of the non-coding sequences provided in SEQ ID NOs:67-68, or a part thereof.

Accordingly, said isolated and complementary polynucleotide is selected from the group comprising or consisting of an antisense oligonucleotide, a gapmer, a short interfering RNA, a short hairpin RNA, a peptide and a CRISPR-Cas. Ideally the polynucleotide is a CRISPR-Cas and, more ideally still it comprises CRISPR-Cas9.

Those skilled in the art will appreciate that knowledge of the lincRNAs Sghrt and Gas5 or the gene sequence structure of Sghrt and Gas5 enables those skilled in the art to make polynucleotides that prevent, in the case of the former, the function of said lincRNAs or, in the case of the latter, the transcription of said genes to produce said lincRNAs. Indeed, online tools are available for this purpose such as BLAST (Basic Local Alignment Search Tool) i.e. an algorithm for comparing primary biological sequence information, such as the nucleotides of RNA/DNA sequences and identify those sequences that resemble the query sequence above a certain threshold.

In a preferred embodiment of the invention said isolated and complementary polynucleotide shares at least about 75% and, in ascending order of preference, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 97%, 99% and 100% sequence identity with the polynucleotide of i) or ii).

The skilled person will appreciate that homologues, orthologues or functional derivatives of the polynucleotide will also find use in the context of the present invention. Thus, for instance polynucleotides which include one or more additions, deletions, substitutions or the like are encompassed by the present invention. In addition, it may be possible to replace one nucleotide with another of similar “type”.

Thus, the term “homologue/homologous” as used herein refers to sequences which have a sequence with at least 75% etc. homology or similarity or identity to/with the claimed polynucleotide sequence.

In yet a further preferred embodiment of the invention said polynucleotide has a sequence selected from the group comprising or consisting of:

Sghrt miR RNAi
(SEQ ID No. 57)
GGGTCTTTGCCTGGGTTTGTT;
Sghrt miR RNAi
(SEQ ID No. 58)
TGGAATGTATCTGGCTCAGAA;
Sghrt sgRNA1
(SEQ ID No. 61)
TTTCGTCTGAGAGTCGGCTG;
Sghrt sgRNA2
(SEQ ID No. 62)
ACCAGGTAGCCACTGACCGT;
Sghrt KD:
(SEQ ID NO: 64)
TTCGGAACTTGAAGGA;
Gas5 miR RNAi
(SEQ ID No. 55)
AGGTATGCAATTTCCTGAGTA;
Gas5 miR RNAi
(SEQ ID No. 56)
CTCTGTGATGGGACATCTTGT;
Gas5 sgRNA1
(SEQ ID No. 59)
GGAGCGAGCGACGTGCCGGA;
and
Gas5 sgRNA2
(SEQ ID No. 60)
CATGCTGAGTCGTCTTTGTC.
Gas5 KD:
(SEQ ID NO: 63)
AGAACTGGAAATAAGA;

According to a further aspect of the invention there is provided a pharmaceutical composition comprising the afore said polynucleotide and a suitable carrier, adjuvant, diluent and/or excipient.

According to a yet further aspect of the invention there is provided a vector comprising or encoding said isolated polynucleotide of the invention.

As used herein, the term “vector” refers to an expression vector, and may be for example in the form of a plasmid, a viral particle, a phage, lipid based vehicle and cell based vehicles. Examples of such delivery vehicles include: biodegradable polymer microspheres, lipid based formulations such as liposome carriers, coating the construct onto colloidal gold particles, lipopolysaccharides, polypeptides, polysaccharides, pegylation of viral vehicles etc. Further, such vectors may also include: adenoviruses, retroviruses, lentiviruses, adeno-associated viruses, herpesviruses, vaccinia viruses, foamy viruses, cytomegaloviruses, Semliki forest virus, poxviruses, pseudorabies, RNA virus vector and DNA virus vector. Such viral vectors are well known in the art. Further the invention includes bacterial plasmids, phage DNA, baculovirus, yeast plasmids, vectors derived from combinations of plasmids and phage DNA. Large numbers of suitable vectors are known to those of skill in the art and are commercially available.

However, any other vector may be used as long as it is replicable and viable in the host. The polynucleotide sequence, preferably the DNA sequence in the vector is operatively linked to an appropriate expression control sequence(s) (promoter) to direct mRNA synthesis. As representative examples of such promoters, one can mention prokaryotic or eukaryotic promoters such as CMV immediate early, HSV thymidine kinase, early and late SV40, LTRs from retrovirus, and mouse metallothionein-I. The expression vector also contains a ribosome-binding site for translation initiation and a transcription vector. The vector may also include appropriate sequences for amplifying expression.

In addition, the vector preferably contains one or more selectable marker genes to provide a phenotypic trait for selection of transformed host cells such as dihydrofolate reductase or neomycin resistance for eukaryotic cell culture, or such as tetracycline or ampicillin resistance in E. coli.

According to a yet further aspect of the invention there is provided a host cell transformed with or transfected with or comprising the said vector.

As used herein, the term “host cell” relates to a host cell, which has been transduced, transformed or transfected with the polynucleotide or with the vector described previously. As representative examples of an appropriate host cell, one can use a bacterial cell, such as E. coli, Streptomyces, Salmonella typhimurium, fungal cell such as yeast, insect cell such as Sf9, animal cell such as CHO or COS, or a plant cell etc. The selection of an appropriate host is deemed to be within the scope of those skilled in the art from the teachings herein. Preferably, said host cell is an animal cell, and most preferably a human cell.

According to a yet further aspect of the invention there is provided a method for producing the isolated polynucleotide of the invention, the method comprising:
culturing the host cell of the invention under suitable conditions to permit production of the polynucleotide; and
recovering the polynucleotide so produced.

According to a further aspect of the invention there is provided said polynucleotide of the invention for use as a medicament.

According to a yet further aspect of the invention there is provided said polynucleotide of the invention for use in the prevention or treatment of cardiac disease.

According to a yet further aspect of the invention there is provided said polynucleotide of the invention for use in the manufacture of a medicament to treat cardiac disease.

According to a further aspect of the invention, there is provided a method for preventing or treating cardiac disease comprising administering an effective amount of said polynucleotide of the invention to an individual to be treated.

Ideally said individual is a mammal and most ideally human.

Reference herein to a cardiac disease includes, but is not limited to, myocardial infarction, heart failure, coronary artery disease (narrowing of the arteries, heart attack, abnormal heart rhythms, arrhythmias, heart failure, heart valve disease, congenital heart disease, heart muscle disease (cardiomyopathy), pericardial disease, aorta disease, marfan syndrome, genetic cardiomyopathy, non-genetic cardiomyopathy, cardiac hypertrophy, pressure overload-induced cardiac dysfunction and damaged heart tissues.

In a preferred embodiment of the invention said preventing or treating cardiac disease comprises rescuing or improving heart function or at least partially rescuing or improving one or more of the following: ejection fraction, left ventricle wall thickness, right ventricle wall thickness, left ventricular wall stress, right ventricular wall stress, ventricular mass, contractile function, cardiac hypertrophy, end diastolic volume, end systolic volume, cardiac output, cardiac index, pulmonary capillary wedge pressure and pulmonary artery pressure.

Reference herein to an “effective amount” of the polynucleotide or a composition comprising same is one that is sufficient to achieve a desired biological effect, in this case cardiac protection and/or cardiac repair. It is understood that the effective dosage will be dependent upon the age, sex, health, and weight of the recipient, kind of concurrent treatment, if any, frequency of treatment, and the nature of the effect desired. Typically the effective amount is determined by those administering the treatment.

Compounds for use in medicine will generally be provided in a pharmaceutical or veterinary composition and therefore according to a yet further aspect of the invention there is provided a pharmaceutical composition comprising a polynucleotide as defined herein and a pharmaceutically acceptable carrier, adjuvant, diluent or excipient.

According to a yet further aspect of the invention there is provided said polynucleotide for use as a cardiac regenerative agent or a cardiac proliferative agent or a cardiac dedifferentiation agent.

According to a further aspect of the invention, there is provided a method for the proliferation, regeneration or dedifferentiation of a heart cell, the method comprising contacting the heart cell with the inhibitor or polynucleotide according to the invention.

In a preferred embodiment the heart cell comprises a cardiomyocyte, ideally, an adult cardiomyocyte and more ideally still, the method is undertaken in vitro, although it may also be practiced in vivo.

According to a further aspect of the invention, there is provided a prognostic or diagnostic method to assess the regenerative or proliferative capacity of heart tissue before, after or during a cardiac treatment regimen comprising: determining the presence or amount of lincRNA(s) Sghrt and/or lincRNA(s) Gas5 in a cardiac sample of said heart tissue; and

where either one or more of lincRNA(s) Sghrt and/or lincRNA(s) Gas5 is present concluding the proliferative capacity of said heart tissue is poor; and where either one or more of lincRNA(s) Sghrt and/or lincRNA(s) Gas5 is absent concluding the proliferative capacity of said heart tissue is good.

In a preferred method of the invention, said determining step involves extracting RNA and performing single nuclear RNA-sequencing then comparing the RNA sequences obtained with any one or more of SEQ ID Nos:1-53 to determine whether any one or more of lincRNA(s) Sghrt and/or Gas5 is present. Ideally, an amplification is undertaken before said RNA-sequencing step.

In an alternative method of the invention said determining step involves assaying for the functional activity of said lincRNAs, for example via use of a competitive binding assay for the lincRNA target.

According to a yet further aspect of the invention there is provided a kit comprising PCR primers for amplifying the polynucleotide of any one of SEQ ID Nos:1, 3, 5, 7, 9, 11, 13, 15, 17, 19, 21, 23, 25, 27, 29, 31, 33, 35, 37, 39, 41, 43, 45, 47, 49, 51-53 and/or probes for hybridizing to said polynucleotide(s).

The invention also extends to a method for screening for a therapeutic agent that can be used to treat or to prevent a heart disorder in an individual, the method comprising:
analyzing a functional expression and/or an expression level of one or more lincRNA(s) Sghrt and/or lincRNA(s) Gas5 in the presence and in the absence of a candidate therapeutic agent; and
determining if the candidate therapeutic agent is a useful therapeutic agent for treating or preventing a heart disorder based on the differences in the functional expression and/or expression level of oneor more lincRNA(s) Sghrt and/or lincRNA(s) Gas5 in the presence of the candidate therapeutic agent and in the absence of the candidate therapeutic agent.

In a preferred method of the invention the candidate therapeutic agent is identified as a useful therapeutic agent for treating or preventing a heart disorder if the functional expression and/or expression level of the polynucleotide is reduced in the presence of the candidate therapeutic agent as compared to in the absence of the candidate therapeutic agent.

According to a yet further aspect of the invention there is provided at least one inhibitor for inhibiting:
a) one or both of Sghrt gene and/or Gas5 gene transcription to produce a Sghrt transcript and/or a Gas 5 transcript;
   comprising an isolated polynucleotide comprising or consisting of a sequence:
i) that is complementary to gDNA Sghrt SEQ ID NO:67, or a part thereof and/or gDNA Gas5 SEQ ID NO:68, or a part thereof; or
ii) a sequence that shares at least 75% identity with the polynucleotide of i).

In a preferred embodiment of this aspect of the invention the inhibitor, ideally but not exclusively, inhibits the function of the Sghrt and/or Gas5 to thus silence the gene and so prevent it from producing a transcript, typically an RNA—of any type—but in particular mRNA or lincRNA. Such inhibitors are known to those skilled in the art. We describe herein Sghrt and Gas5 Knock downs which are CRISPR based i.e. sgRNAs that specifically delete the promoter and first exon of either Gas5 or Sghrt. However, other inhibitors that silence either the Sghrt or Gas5 gene may be used to work the invention. Ideally, the use of the afore inhibitor is used to treat a cardiac disease and so provide for cardiac regeneration or a cardiac proliferation or a cardiac dedifferentiation.

Throughout the description and claims of this specification, the word “comprise” and variations thereof, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith. Moreover, unless stated otherwise, any feature disclosed herein may be replaced by an alternative feature serving the same or a similar purpose.

The invention will now be described by way of example only with reference to the Examples below and to the following Figures wherein:

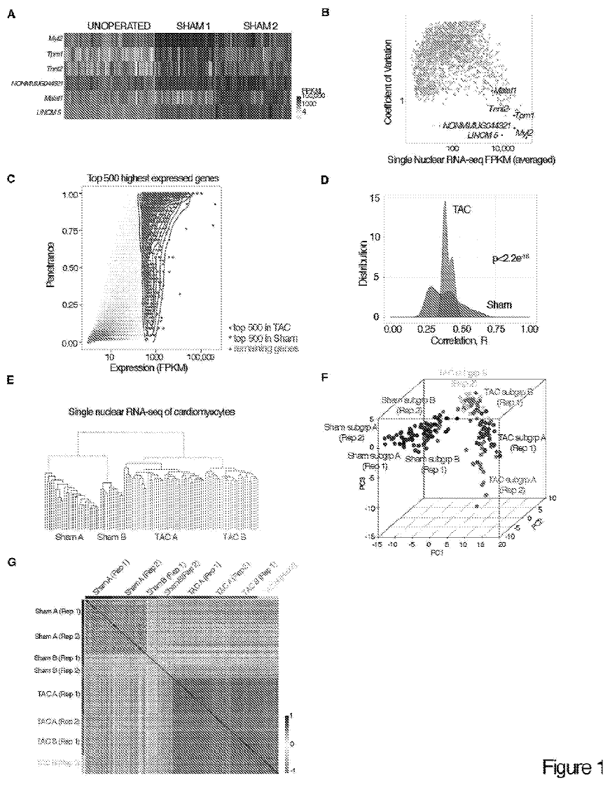

FIG. 1. Shows single nuclear RNA-seq reveals heterogeneity and gene regulatory modules specific to Sham and TAC nuclear subgroups in mouse left ventricle.

A-B, Core cardiac genes that are most highly expressed in every CM nucleus (A) exhibit high expression with low Coefficient of Variation (B).

C, Highly expressed genes in TAC nuclei have higher penetrance than highly expressed genes in Sham nuclei. Spearman's rank correlation (r=0.90, p<2.2e-16) shows good correlation between average expression level and penetrance.

D, Density distribution of correlation shows higher correlation in TAC nuclei than in Sham nuclei. p<2.2e-16 from Mann Whitney U test.

E-F, Unsupervised hierarchical clustering (E) and PCA (F) of single nuclear RNA-seq of CM reveal that CM nuclei accurately segregate into clusters specific to Sham or TAC subgroups (subgroup A, B) and is replicated across biological repeats (Rep).

G, Ranked Spearman correlation plot shows higher correlation in TAC nuclei than in Sham nuclei, which is replicated across biological repeats (Rep).

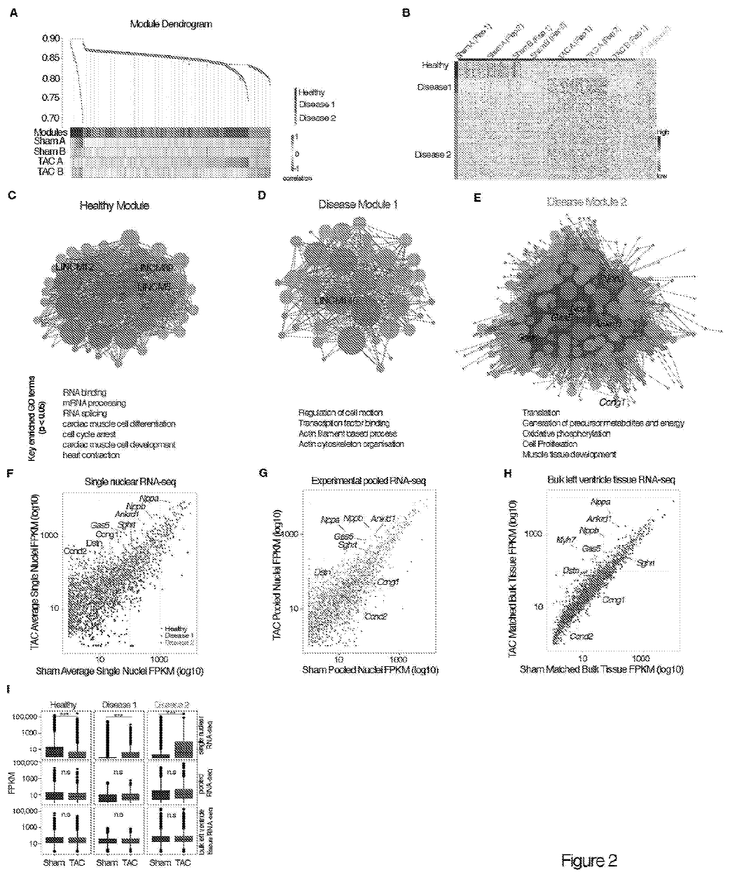

FIG. 2. Shows WGCNA of single CM nuclear RNA-seq identifies lincRNAs as nodal hubs in gene regulatory networks.

A-B, WGCNA identifies three distinct gene modules (Healthy, Disease 1 and Disease 2) (A) in Sham and TAC nuclei that represent expression signatures of specific Sham or TAC nuclear subgroups (B).

C-E, WGCNA reveals candidate lincRNAs in nodal hubs bearing the highest connectivity with other genes within the gene regulatory network modules. Gas5 and Sghrt are in nodal hubs within Disease Module 2 (E) and highly correlated with expression of other genes in the network such as Nppa, Dstn, Ccng1, Ccnd2. Size of bubbles represent strength and significance of connectivity. Key enriched Gene Ontology (GO) terms are listed for each module (p<0.05 Fischer's exact test).

F-H, Scatterplots showing the expression of genes from the 3 gene modules at the single-nuclear level (F), at pooled nuclei level (G) and matched bulk left ventricle tissue RNA-seq (H).

I, Significant differential expression of genes from the 3 gene modules between Sham and TAC samples is detected only by single nuclear RNA-seq, and not by pooled nuclei or bulk tissue RNA-seq.

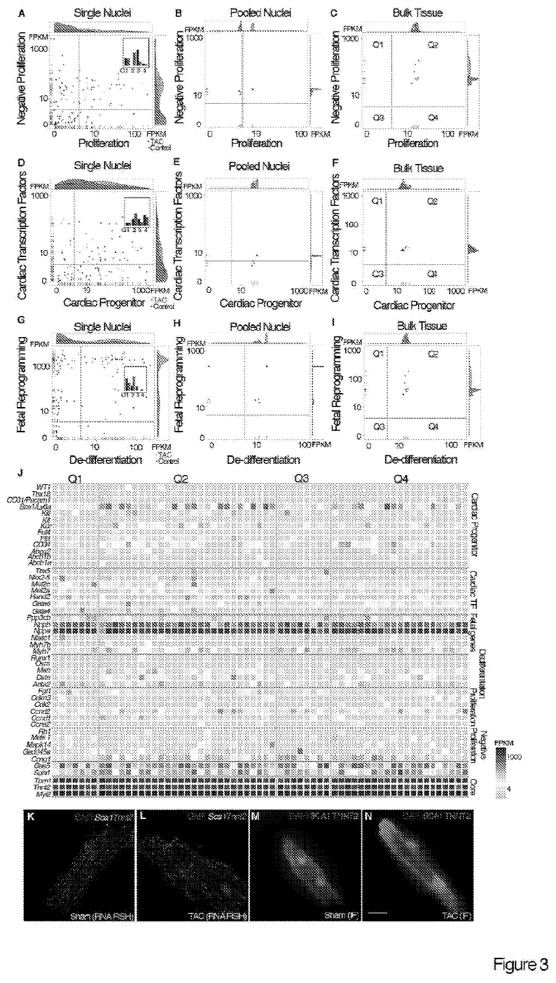

FIG. 3. Shows quadrant analyses reveal sub-populations of CM that co-express proliferation, cardiac progenitor, transcription factors and dedifferentiation genes.

A-C, Quadrant analysis for Proliferation vs Negative regulators of proliferation genes identifies increased co-expression in individual TAC nuclei (Q2; 44.4%; p=3.237e-07 Fischer's exact test), only detectable by single nuclear RNA-seq

(A), and not in pooled nuclei (B) or matched bulk left ventricle RNA-seq (C). Inset: histogram of nuclei distributed across quadrants.

D-F, Quadrant analysis for Cardiac Progenitor vs Cardiac Transcription Factor gene expression shows increased co-expression upon TAC stress in single CM nuclei (D) (Q2; 42.9%; p=2.548e-05 Fischer's exact test), again not detectable in pooled nuclei or bulk tissue RNA-seq (E, F).

G-I, Increased co-expression of fetal reprogramming genes and dedifferentiation markers under TAC stress only detected in single nuclear RNA-seq (G) (Q2; 58.73%; p=0.001371 Fischer's exact test) and not in non-single approaches (H-I).

J, High co-expression of cardiac progenitors, cardiac transcription factors, dedifferentiation, proliferation and negative proliferation markers is confined to single nuclear TAC samples in Q2 and Q4.

K-L, Single molecule RNA FISH shows Sca1 upregulation and co-expression of Tnnt2 in isolated adult mouse CMs from TAC hearts (L) compared to Sham (K). Number of Sca1+Sham CMs: 5/13; Sca1+ TAC CMs: 38/55; all together from 2 Sham and 3 TAC biological replicates.

M-N, Immunofluorescence confirms increase in cell surface SCA1 protein expression in TAC CMs (N) compared to Sham (M). Number of SCA1+Sham CMs: 8/23; SCA1+ TAC CMs: 43/66; all together from 2 Sham and 3 TAC biological replicates. Scale bar represents 20 μm.

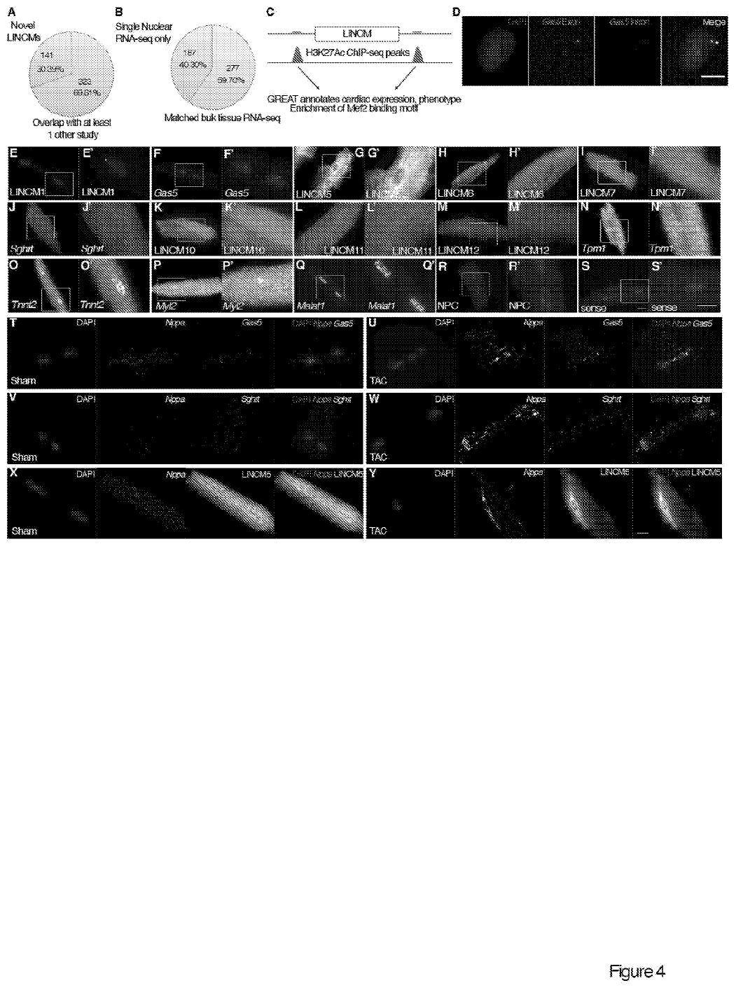

FIG. 4. Shows single molecule RNA FISH validates cellular expression of LINCMs in isolated adult mouse CMs.

A, Single nuclear RNA-seq identifies 141 novel lincRNAs in nuclei of CMs (LINCMs) that are not in current public databases (ENSEMBL, NONCODE) nor published cardiac transcriptome datasets.

B, Single nuclear RNA-seq identifies LINCMs that are not detectable in matched left ventricle bulk tissue RNA-seq, explained by the dilution of reads in cytoplasmic mRNA pool.

C, Active H3K27Ac enhancer chromatin regions proximal to LINCMs are enriched in MEF2 transcription factor binding motif and functionally annotated by GREAT analysis to have cardiac expression and phenotypes.

D, Sites of active transcription demonstrated by co-localization of exonic and intronic probes (asterisk) in nucleus. Scale bar represents 5 μm.

E-M′, Single molecule RNA FISH validates the expression of LINCMs in isolated adult mouse CMs.

N-Q′, Positive controls for highly abundant core genes Tpm1, Tnnt2, Myl2 and Malat1.

R-S′, Negative controls with No Probe Control (NPC) (R,R′) and a sense probe (S,S′) to confirm signal specificity. Scale-bar represents 10 μm. E′-S′, Zoom-in view of same images in E-S.

T-U, Gas5 is upregulated in TAC CM and co-localizes with perinuclear Nppa transcripts.

V-W, Sghrt is upregulated and localizes to the cytoplasm of TAC CM.

X-Y, LINCM5 is downregulated in TAC CM. Scale bar represents 10 μm.

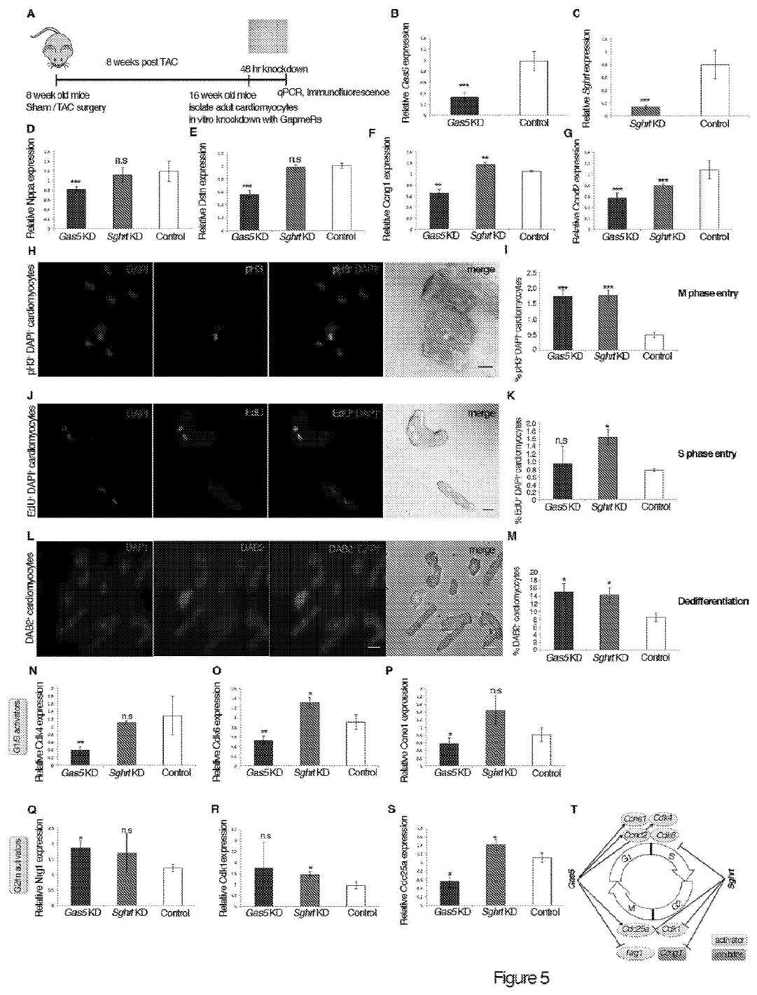

FIG. 5. Shows Gas5 and Sghrt transcriptionally regulate S phase and M phase entry of adult CMs during TAC stress.

A, Strategy to knockdown Gas5 or Sghrt independently in isolated adult TAC CMs in vitro.

B-C, In vitro knockdown of Gas5 or Sghrt in adult mouse CMs using GapmeRs is efficient and reproducible across biological replicates. N=5 biological replicates.

D-G, Gas5 knockdown in TAC CMs results in significant reduction of Nppa, Dstn, Ccng1 and Ccnd2 expression. Sghrt knockdown in TAC CMs results in significant increase in Ccng1 and reduction in Ccnd2 expression.

H, Representative image of pH3+ DAPI+ adult CM indicating M phase re-entry. Scale bar 20 μm.

I, Knockdown of either Gas5 or Sghrt in isolated adult mouse TAC CMs increases the percentage of CMs with pH3+ nuclei. mean±s.e.m. Over 3,600 CMs were imaged and counted from N=3 biological replicates of TAC-operated mice.

J, Representative image of EdU+ DAPI+ CM with nascent DNA synthesis in S phase re-entry. Scale bar 20 μm.

K, Knockdown of Sghrt, but not Gas5, in isolated adult mouse TAC CMs increases the percentage of CMs with EdU+ DAPI+ nuclei. mean±s.e.m. Over 3,600 CMs were imaged and counted from N=3 biological replicates of TAC-operated mice.

L, Representative image of DAB2+ CM expressing the dedifferentiation marker.

M, Knockdown of either Gas5 or Sghrt in isolated adult mouse TAC CMs increases the percentage of DAB2+ CMs.

N-P, Gas5 knockdown reduces expression of G1/S phase activators (Cdk4, Cdk6, Ccne1, Ccnd2), but Sghrt knockdown results in significant increase in Cdk6 expression.

Q, Gas5 knockdown increases Nrg1 expression.

R-S, Knockdown of Sghrt, but not Gas5, increases expression of G2/M phase activators (Cdk1, Cdc25a).

T, Schematic of transcriptional regulation of G1/S phase and G2/M phase by Gas5 and Sghrt.

All gene expression analysis is normalized to housekeeping transcript (Rplp0). *P<0.05, **P<0.01, ***P<0.001, n.s. not significant; mean±s.e.m. All representative of N=3-5 biological replicates of TAC-operated mice.

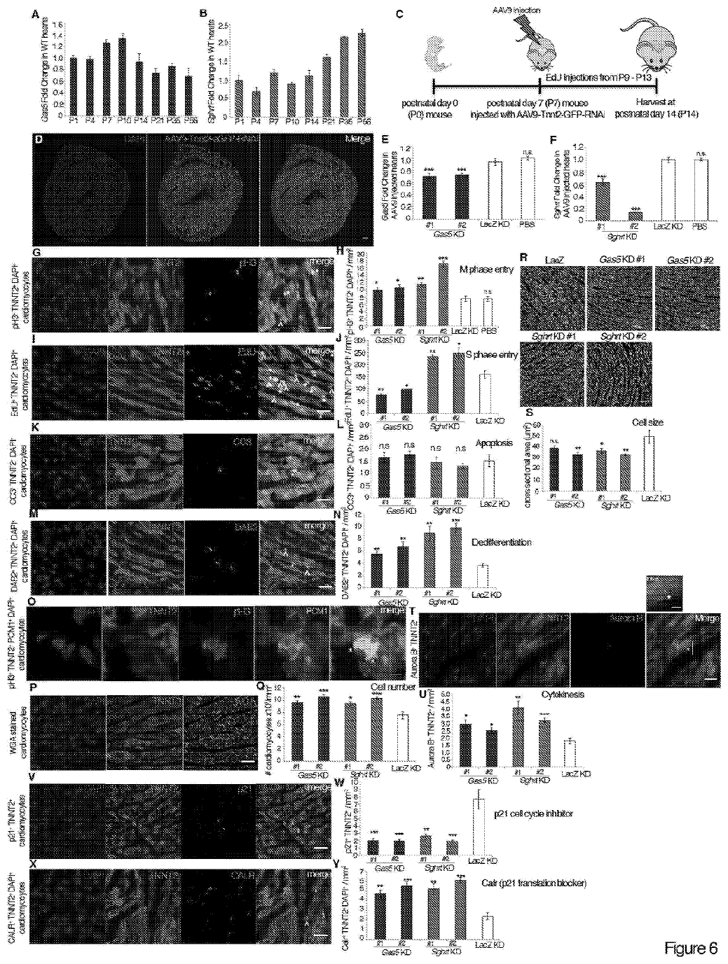

FIG. 6. Shows Gas5 and Sghrt regulate S phase entry, M phase entry and proliferation of CM in vivo.

A-B, Expression of endogenous Gas5 (A) and Sghrt (B) in mouse heart across post-natal stages. Gas5 expression peaks at P7-P10 and reduces with age (A). Sghrt expression peaks at P7 and gradually increases with age (B). The increase in expression of Gas5 and Sghrt at P7 coincides with the endogenous loss of CM proliferation potential.

C, Strategy to knockdown Gas5 or Sghrt specifically in mouse CMs in vivo D, Representative images of histological heart section of AAV9-TNNT2-eGFP-RNAi injected mouse showing strong eGFP reporter expression. Scale bar 100 μm.

E-F, In vivo knockdown of LINCM is significant and reproducible across biological replicates. Animals injected for AAV9-TNNT2-eGFP-RNAi experiments in E-Q: N=8 mice Gas5 KD #1, N=7 mice Gas5 KD #2, N=7 mice Sghrt KD #1, N=8 mice Sghrt KD #2, N=5 mice LacZ KD, N=8 mice PBS.

G, Representative image of pH3+ TNNT2+ DAPI+ CM (asterisks) with M phase entry in vivo. Arrowhead indicates pH3+ DAPI+ TNNT2− non-CMs not included in quantification. Scale bar 30 μm.

H, Knockdown of Gas5 or Sghrt significantly increased M phase entry of CMs in vivo.

I, Representative image of EdU+ TNNT2+ DAPI+ CM (asterisks) with S phase entry in vivo. Arrowheads indicate EdU+ DAPI+ TNNT2− non-CMs not included in quantification. Scale bar 30 μm.

J, Gas5 knockdown significantly reduced S phase entry, whereas Sghrt knockdown significantly increased S phase entry of CMs in vivo.

K, Representative image of CC3+ TNNT2+ DAPI+ CM (asterisk) with apoptosis in vivo. Scale bar 30 μm.

L, No significant change in apoptosis induced by knockdown of Gas5 or Sghrt in vivo.

M, Representative image of DAB2+ TNNT2+ DAPI+ CM (asterisk) with dedifferentiation in vivo. Arrowheads indicate DAB2+ DAPI+ TNNT2− non-CMs not included in quantification. Scale bar 30 μm.

N, Knockdown of Gas5 or Sghrt significantly increased CM dedifferentiation in vivo.

O, Representative image of two adjacent pH3+ PCM1+ TNNT2+ DAPI+ CM nuclei (asterisks) undergoing M phase in vivo. Scale bar 3 μm.

P, Representative image of WGA stained CMs for quantification. Scale bar 30 μm.

Q, Significant increase in number of CMs per mm2 after knockdown of Gas5 or Sghrt in vivo.

R, Representative images of WGA stained CMs in histological sections showing reduced cross sectional area of CMs after Gas5 KD or Sghrt KD compared to LacZ KD. Scale bar 50 μm.

S, Significant reduction of cross sectional area (μm2) in Gas5 KD or Sghrt KD compared to LacZ KD suggests smaller cell size after knockdown.

T, Representative image of Aurora B+ TNNT2+ CMs (asterisk) detecting cytokinesis in vivo. Scale bar 20 μm, inset scale bar 5 μm.

U, Significant increase in Aurora B+ TNNT2+ CMs after knockdown of Gas5 or Sghrt in vivo.

V, Representative image of p21+ TNNT2+ CMs (asterisks) expressing the p21 cell cycle inhibitor. Scale bar 30 μm.

W, Significant reduction in p21+ TNNT2+ CMs after knockdown of Gas5 or Sghrt in vivo.

X, Representative image of CALR+ DAPI+ TNNT2+ CMs (asterisks) expressing Calreticulin that blocks p21 protein translation. Arrowheads indicate CALR+ DAPI+ TNNT2− non-CMs not included in quantification. Scale bar 30 μm.

Y, Significant increase in CALR+ DAPI+ TNNT2+ CMs after knockdown of Gas5 or Sghrt in vivo.

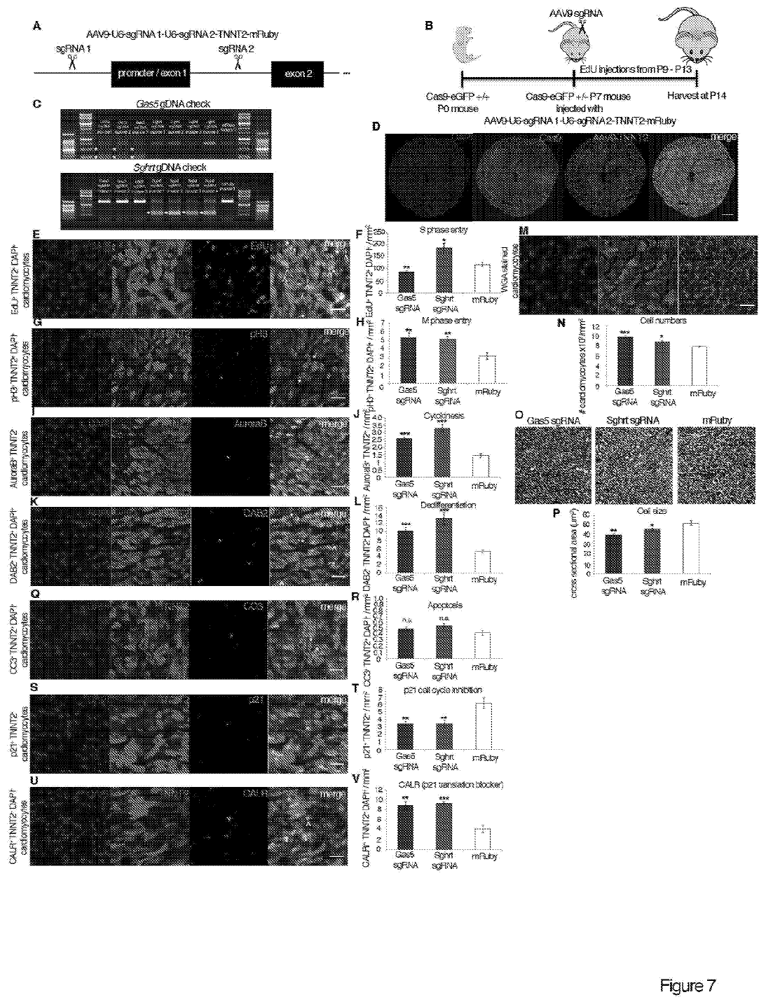

FIG. 7. AAV9-CRISPR Cas9 mediated genomic deletions in vivo recapitulates Gas5 and Sghrt regulated proliferation of CM in vivo.

A, Schematic design of sgRNA pairs to delete genomic regions containing promoter and first exon of Gas5 or Sghrt.

B, Strategy to generate genomic deletions of Gas5 or Sghrt specifically in mouse heart in vivo.

C, AAV9-CRISPR Cas9 cuts specifically at target genomic regions in mouse heart in vivo. Truncated PCR amplicons (asterisks) were cloned and sequenced for confirmation. Negative control (AAV9-TNNT2-mRuby2) and reciprocal genomic regions confirmed the absence of crossover off-target editing.

D, Representative image of AAV9-injected mouse heart showing robust co-expression of Cas9-eGFP transgene, with U6-sgRNA-TNNT2-mRuby2 in vivo. Animals injected for AAV9-CRISPR Cas9 experiments in E-V: N=8 mice Gas5 sgRNA; N=10 mice Sghrt sgRNA; N=7 mice mRuby2 controls.

E, Representative image of EdU+ TNNT2+ DAPI+ CM (asterisks) with S phase entry in vivo. Arrowheads indicate EdU+ DAPI+ TNNT2− non-CMs not included in quantification. Scale bar 30 μm.

F, Gas5 AAV9-CRISPR Cas9 KD significantly reduced S phase entry, whereas Sghrt AAV9-CRISPR Cas9 KD significantly increased S phase entry of CMs in vivo.

G, Representative image of pH3+ TNNT2+ DAPI+ CM (asterisks) with M phase entry in vivo. Scale bar 30 μm.

H, Knockdown of Gas5 or Sghrt by AAV9-CRISPR Cas9 significantly increased M phase entry of CMs in vivo.

I, Representative image of Aurora B+ TNNT2+ CMs (asterisk) evident for cytokinesis in vivo. Scale bar 30 μm.

J, Significant increase in Aurora B+ TNNT2+ CMs after Gas5 or Sghrt KD by AAV9-CRISPR Cas9 in vivo.

K, Representative image of DAB2+ TNNT2+ DAPI+ CM (asterisks) with dedifferentiation in vivo. Arrowheads indicate DAB2+ DAPI+ TNNT2− non-CMs not included in quantification. Scale bar 30 μm.

L, Knockdown of Gas5 or Sghrt by AAV9-CRISPR Cas9 significantly increased CM dedifferentiation in vivo.

M, Representative image of WGA stained CMs for quantification. Scale bar 30 μm.

N, Significant increase in number of CMs per mm2 after Gas5 or Sghrt KD by AAV9-CRISPR Cas9 in vivo.

O, Representative images of WGA stained CMs in histological sections showing reduced cross sectional area of CMs after Gas5 or Sghrt KD by AAV9-CRISPR Cas9 in vivo. Scale bar 50μm.

P, Significant reduction of cross sectional area (pmt) suggests smaller cell size after Gas5 or Sghrt KD by AAV9-CRISPR Cas9 in vivo.

Q, Representative image of CC3+ TNNT2+ DAPI+ CM (asterisk) with apoptosis in vivo. Arrowhead indicates CC3+ TNNT2− non-CMs not included in quantification. Scale bar 30 μm.

R, No significant change in apoptosis induced after Gas5 or Sghrt KD by AAV9-CRISPR Cas9 in vivo.

S, Representative image of p21+ TNNT2+ CMs (asterisk) expressing the p21 cell cycle inhibitor. Scale bar 30μm.

T, Significant reduction in p21+ TNNT2+ CMs after Gas5 or Sghrt KD by AAV9-CRISPR Cas9 in vivo.

U, Representative image of CALR+ DAPI+ TNNT2+ CMs (asterisks) expressing Calreticulin that blocks p21 protein translation. Arrowheads indicate CALR+ DAPI+ TNNT2− non-CMs not included in quantification. Scale bar 30 μm.

V, Significant increase in CALR+ DAPI+ TNNT2+ CMs after Gas5 or Sghrt KD by AAV9-CRISPR Cas9 in vivo.

Images in G, I, K, Q, S, U were stained with Alexa647 secondary antibodies to avoid spectral overlap with mRuby2 or eGFP.

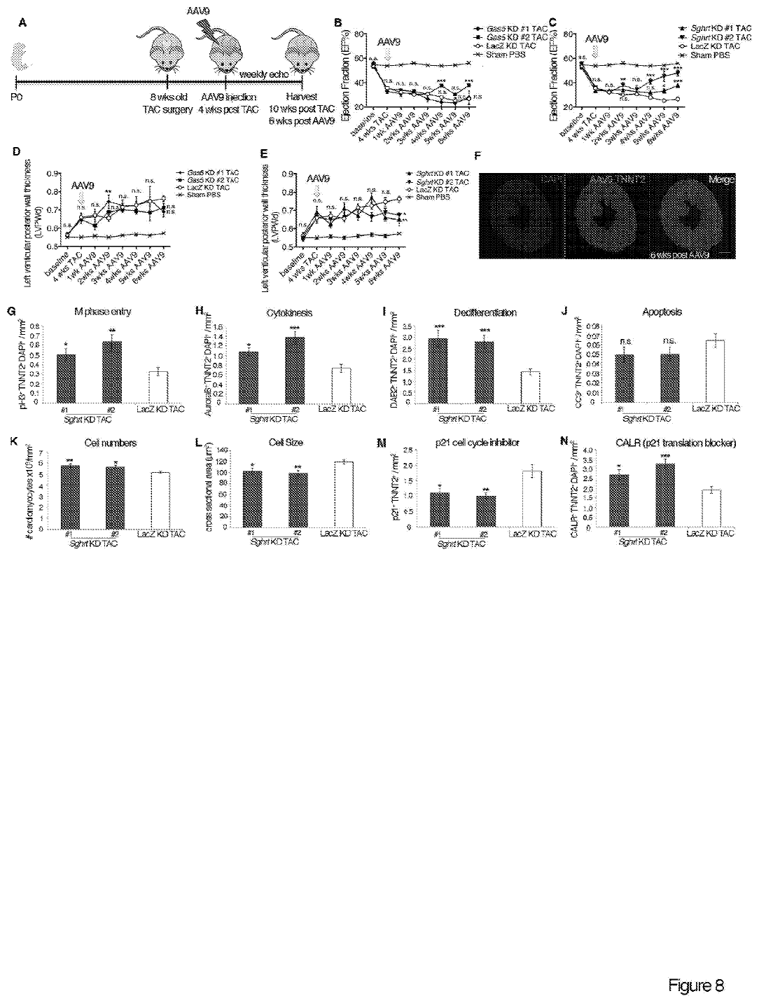

FIG. 8. Rescue of heart function in TAC mouse model of heart failure after onset of hypertrophy by knockdown of Sghrt in vivo.

A, Strategy to knockdown Gas5 or Sghrt in vivo at 4 weeks post TAC (after onset of hypertrophy and reduced ejection fraction) followed by weekly monitoring by echocardiography up to 6 weeks post AAV9 injection. Animals injected for TAC rescue experiments: N=6 mice Gas5 KD #1 TAC, N=6 mice Gas5 KD #2 TAC, N=5 mice Sghrt KD #1 TAC, N=7 mice Sghrt KD #2 TAC, N=9 mice LacZ KD TAC, N=14 mice PBS injected Sham.

B-C, Gas5 KD at 4 weeks post-TAC did not result in significant rescue of ejection fraction (EF %) (B), whereas Sghrt KD at 4 wks post-TAC resulted in partial rescue of EF % starting from 5 weeks post AAV9 injection (C).

D-E, Gas5 KD at 4 weeks post-TAC showed no significant effect on hypertrophy as measured by left ventricular posterior wall thickness in diastole (LVPWd) (D), whereas Sghrt KD at 4 weeks post-TAC resulted in significant regression of LVPWd starting at 6 weeks post AAV9 injection (E).

F, Representative image of AAV9-TNNT2-eGFP-RNAi injected TAC mouse heart showing robust expression in vivo even after 6 weeks post AAV9 injection. Scale bar 1000 μm.

G, Sghrt KD resulted in significant increase in M phase entry of CMs (pH3+ TNNT2+ DAPI+) at 6 weeks post-AAV9.

H, Sghrt KD resulted in significant increase in cytokinesis of CMs (AuroraB+ TNNT2+) at 6 weeks post-AAV9.

I, Sghrt KD resulted in significant increase in dedifferentiation of CMs (DAB2+ TNNT2+ DAPI+) at 6 weeks post-AAV9.

J, Sghrt KD had no significant effect on CM apoptosis (CC3+ TNNT2+ DAPI+) at 6 weeks post-AAV9.

K, Sghrt KD resulted in significant number of CMs per mm2 at 6 weeks post-AAV9.

L, Sghrt KD resulted in significant reduction in cross sectional area of CMs (pmt) at 6 weeks post-AAV9.

M, Sghrt KD resulted in significant reduction of p21 cell cycle inhibitor protein levels in CMs (p21+ TNNT2+) at 6 weeks post-AAV9.

N, Sghrt KD resulted in significant increase of Calreticulin that blocks p21 translation in CMs (CALR+ TNNT2+ DAPI+) at 6 weeks post-AAV9.

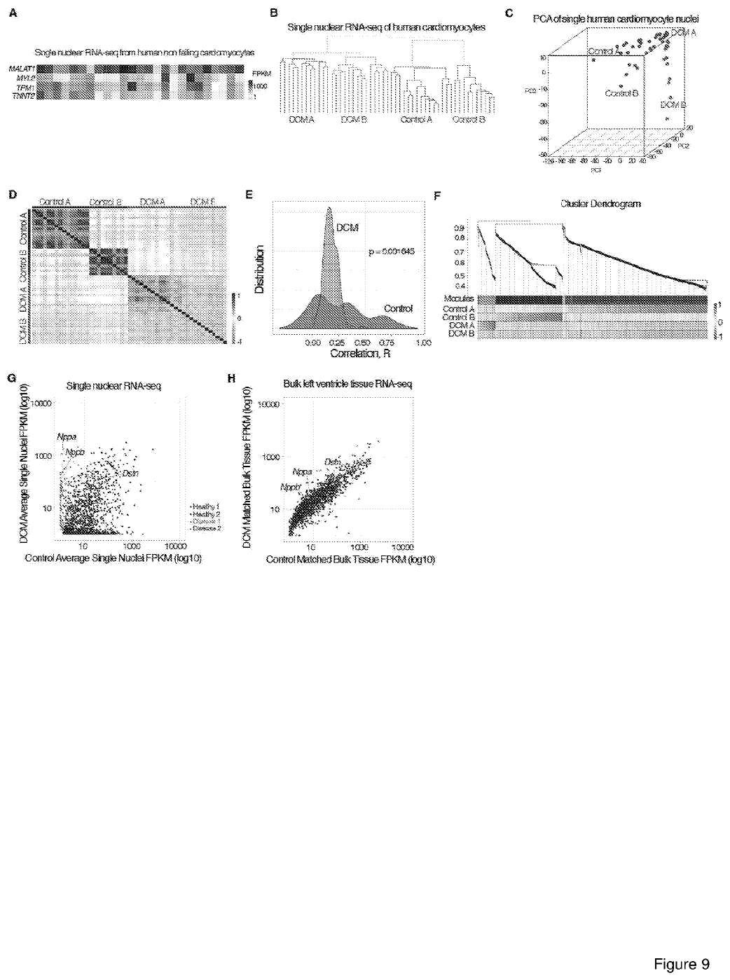

FIG. 9 (Supplementary FIG. 3). Human single nuclear RNA-seq of cardiomyocytes is similar to mouse single nuclear RNA-seq.

A, Core cardiac genes in human CMs are similar to mouse.

B-D, Unsupervised hierarchical clustering (B), PCA (C) and Spearman correlation analysis (D) produced 2 distinct subgroups in each of Control and Dilated Cardiomyopathy (DCM) nuclei.

E, Density distribution of correlation shows narrower distribution for DCM nuclei compared to control. P value from Mann Whitney U test.

F, WGCNA identifies gene modules (Healthy 1, Healthy 2, Disease 1, Disease 2) that are specific for DCM or control nuclear subgroups.

G-H, Classifiers from human gene modules show differential expression at single nuclear level (G), but not in matched bulk left ventricle RNA-seq (H).

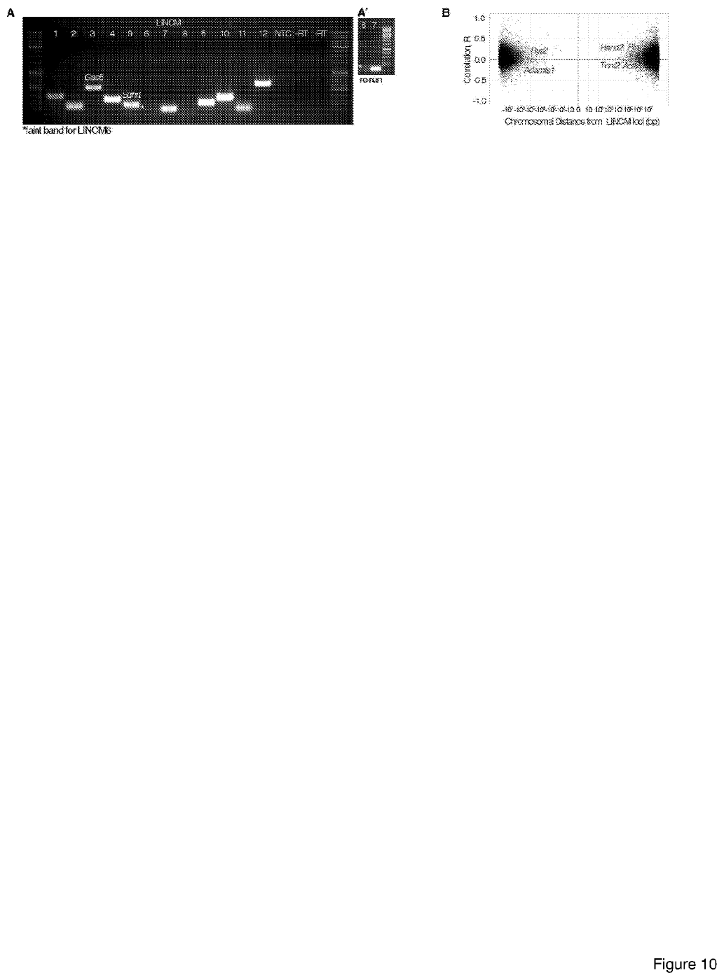

FIG. 10 (Supplementary FIG. 5). Validation of LINCMs in heart by RT-PCR

A-A′, Successful amplification of LINCMs by RT-PCR on DNAse treated RNA to exclude genomic DNA contamination. Right panel, re-run of LINCM6* shows a faint band of correct size on a separate gel (A′). Products were gel extracted and Sanger sequenced to confirm identity.

B, Correlation of gene expression increased with increasing linear chromosomal distance away from LINCM loci.

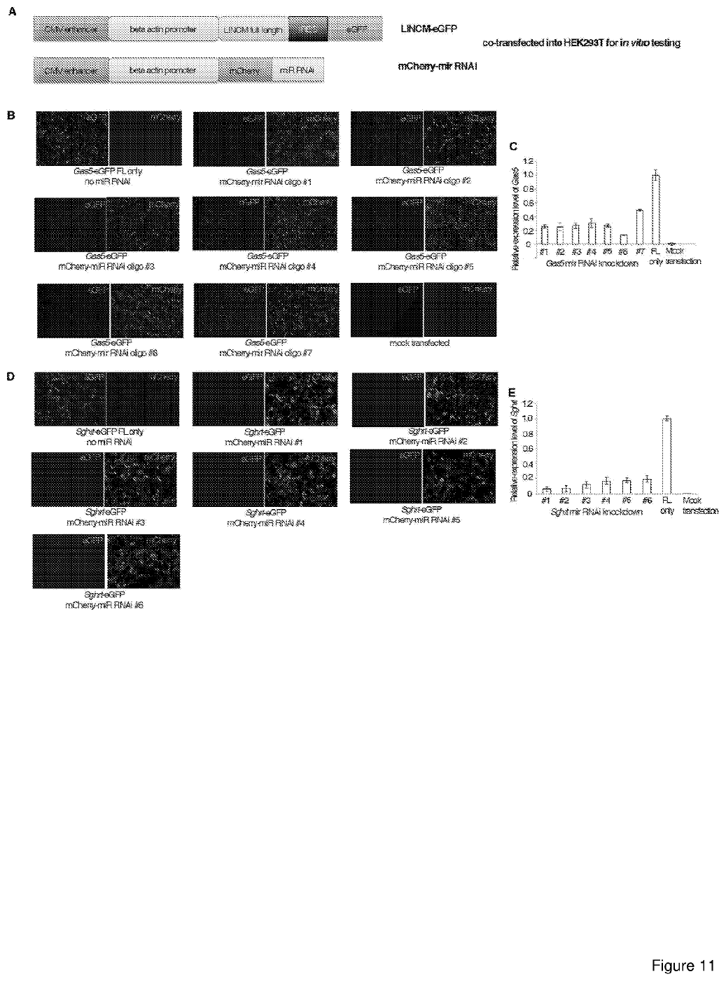

FIG. 11 (Supplementary FIG. 6). In vitro testing of LINCM knockdown efficiency

A, Plasmids used for co-transfection of full length LINCMs (FL) tagged with IRES-eGFP and mCherry-miR RNAi in HEK293T cells for in vitro testing of knockdown efficiency

B-C, Co-transfection of full length Gas5-IRES-eGFP with mCherry-miR RNAi oligos identifies Gas5 oligo #1 and Gas5 oligo #6 (thereafter known as Gas5 KD #2 in FIG. 6) with best knockdown efficiency by fluorescence (B) and validated by qPCR (C).

D-E, Co-transfection of full length Sghrt-IRES-eGFP with mCherry-miR RNAi oligos identifies Sghrt oligo #1 and Sghrt oligo #2 with best knockdown efficiency by fluorescence (B) and validated by qPCR (C).

Full length LINCM-eGFP (FL) alone gave strong signal in for LINCM-eGFP but not for mCherry-miR RNAi.

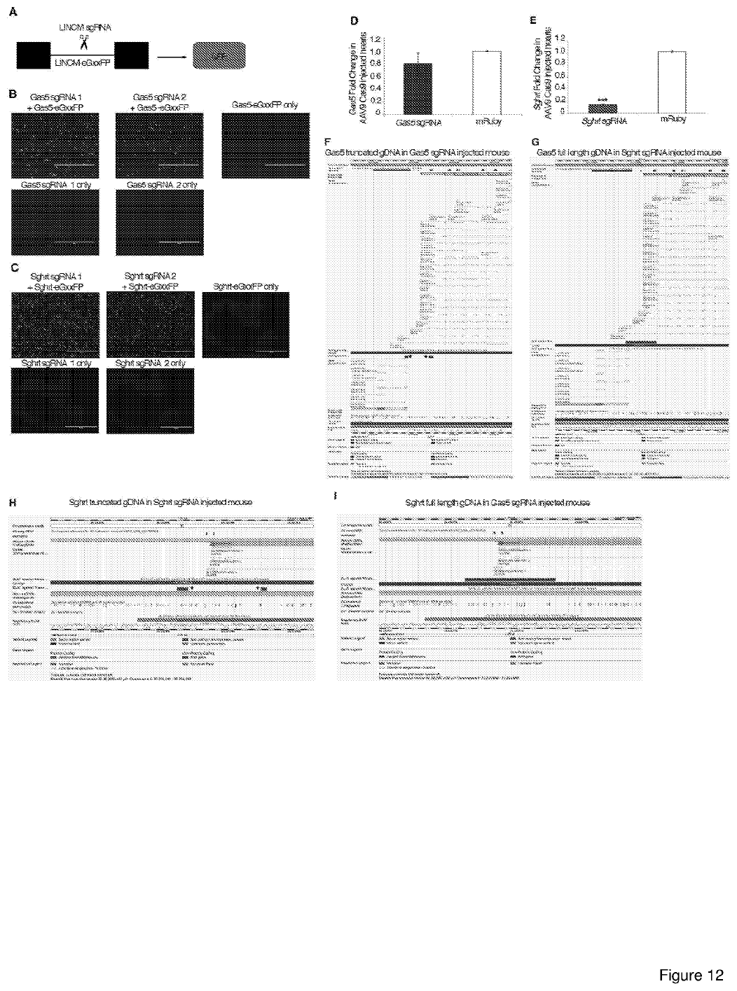

FIG. 12 (Supplementary FIG. 8). In vitro and in vivo validations of CRISPR Cas9 generated genomic deletions of Gas5 or Sghrt.

A, Schematic drawing of pCAG-EGxxFP complementation assay used for in vitro testing of sgRNA against Gas5 or Sghrt target genomic regions. Successful deletions of target genomic regions result in the reconstitution of eGFP fluorescence.

B-C, Representative images of in vitro testing of sgRNAs against Gas5 or Sghrt genomic regions. Negative controls consist of sgRNA only and LINCM-EGxxFP only that are non-fluorescent.

D-E, Validation of reduction in Gas5 or Sghrt transcript levels in resected apex of mouse hearts after AAV9-CRISPR Cas9 mediated genomic deletions in vivo.

F-G, ENSEMBL BLAT of TOPO cloned and Sanger sequenced genomic bands show clear deletions (asterisks) at Gas5 target region in Gas5 sgRNA injected mice (F) but not in Sghrt sgRNA injected mice (G).

H-I, ENSEMBL BLAT of TOPO cloned and Sanger sequenced genomic bands show clear deletions (asterisks) at Sghrt target region in Sghrt sgRNA injected mice (H) but not in Gas5 sgRNA injected mice (I).

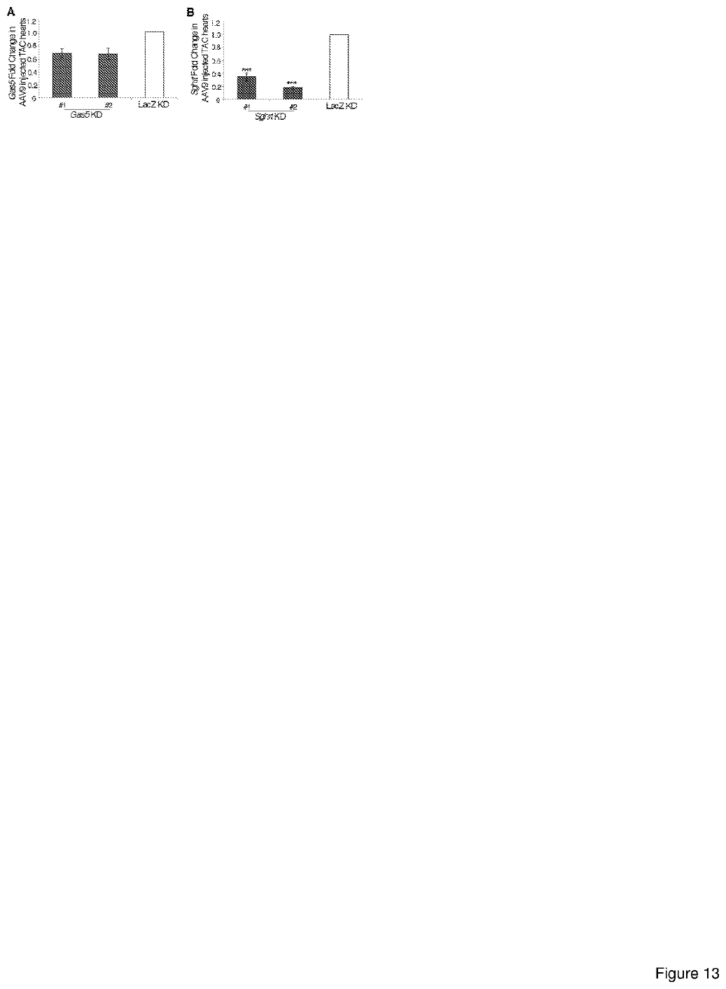

FIG. 13 (Supplementary FIG. 9). Validation of knockdown in TAC mouse hearts at 6 weeks post-AAV9 injection.

A-B, Knockdown of Gas5 (A) or Sghrt (B) transcript levels is observed even after 6 weeks post-AAV9 injection in TAC mouse hearts.

### METHODS AND MATERIALS

### EXPERIMENTAL PROCEDURES

Single Nuclear RNA-Seq Library Preparation

Single nuclei were isolated from snap-frozen mouse and human left ventricle and processed by mechanical dissociation at 4000 Hz (4×20 s pulses) in Lysonator™ cartridges (SG Microlab devices) and counterstained with DAPI. CM nuclei were stained with PCM1 antibody (1:500, HPA023374, Sigma), secondary anti-rabbit Alexa 488 or Alexa 568 antibody, and captured individually using C1 Single Cell Auto Prep system (10-17 uM mRNA seq chip, Fluidigm). Automated imaging of captured nuclei was performed on an inverted microscope (Olympus) with 10× objective (Olympus) and CCD camera (Axiocam MR3, Zeiss) to confirm the identity of wells containing only single PCM1+ CM captured. PCM1+ CM nuclear RNA-seq libraries were prepared using Nextera XT DNA sample preparation kit (Illumina). Each sample was sequenced with paired end 2×101 bp reads on HiSeq 2500 (Illumina).

Human Left Ventricle Samples

Human left ventricles were collected with a protocol approved by the Papworth (Cambridge) Hospital Tissue Bank Review Board and the Cambridgeshire Research Ethics Committee (UK). Written consent was obtained from all individuals according to the Papworth Tissue Bank protocol. DCM left ventricles were from patients undergoing cardiac transplantation for end-stage DCM, harvested as previously described 66,67. Healthy normal left ventricles were from age matched male individuals, through Papworth Hospital or Ethical Tissue (University of Bradford), governed by the UK Human Tissue Authority.

Mouse TAC Surgery and Isolation of Adult Mouse Ventricular CM for FISH

TAC surgery was performed by a standard protocol as previously published68. CM isolations were performed by enzymatic dissociation using perfusion buffer, 37° C. pre-warmed 40 ml enzyme solution (Collagenase II 0.5mg/ml (Worthington), Collagenase IV 0.5 mg/ml (Worthington), and Protease XIV 0.05 mg/ml) at a rate of 2 ml/minute. Enzymes were neutralized with fetal bovine serum (FBS) to final concentration of 5%. Cell suspensions were filtered through 100 μm nylon mesh cell strainers (Thermo Fisher Scientific) and allowed to settle by gravity. Calcium concentration was increased gradually to 1.0 mM. Cells were resuspended in plating medium containing M199 medium with glutamine (2 mM), BDM (10 mM) and FBS (5%), plated onto laminin-coated glass coverslips (#1, Thermo Fisher Scientific) and incubated for 1 hr at 37° C. in a humidified environment with 5% ambient CO2. Non-attached cells were removed by gentle washing in PBS.

Single Molecule RNA FISH

Isolated CM adhered onto laminin coated #1 coverslips (ThermoScientific) were fixed for 10 mins at r.t.p with Fixation Buffer (3.7% formaldehyde in PBS), washed twice in 1× PBS and permeabilized with 70% EtOH at 4° C. for at least an hour. RNA FISH was performed using 20-mer Stellaris Biosearch Probes for LINCMs and core genes conjugated to Quasar 670 or CAL Fluor Red 610. Briefly, cells were washed with Wash Buffer (10% formamide in 2× SSC) prior to overnight 37° C. hybridization with target probes (125 nM) in Hybridization buffer (100 mg/ml Dextran Sulfate, 10% Formamide in 2>SSC). After hybridization, cells were washed in Wash Buffer for 30 mins at 37° C., counterstained with DAPI (5ng/ml in Wash Buffer) for 30 mins at 37° C., and washed in 2×SSC at r.t.p. Coverslips were transferred onto glass slides with mounting medium (Vectashield) and imaging was performed immediately on upright microscope (Nikon Ni-E) with 100× Objective (Nikon) on a cooled CCD/CMOS camera (Qi-1, Qi-2, Nikon).

For the notable exception of Sca1/Tnnt2 RNA FISH co-staining, RNA FISH was performed using 50-mer ZZ ACD RNAScope probes due to the short unique sequence of Sca1 available for probe design and high degree of homology to other members of Ly6 family. Cells were fixed and permeabilized as above in 70% EtOH, washed in 1×PBS and 1× Hybwash buffer for 10 and 30 mins respectively at r.t.p. prior to incubation with 1× Target Probe Mix at 40° C. for 3 hrs. Cells were washed thrice in 1× Hybwash at r.t.p, incubated in 1× Pre Amp Mix for 40 mins at 40° C., washed thrice in 1× Hybwash at r.t.p, incubated in 1× Amp Mix for 30 mins at 40° C., washed twice in 1× Hybwash before incubation in 1× Label Probe Mix (Alexa Fluo 488, ATTO0550) at 40° C. for 25 mins. Cells were washed thrice in 1× Hybwash in dark at r.t.p, counterstained with DAPI (5ng/ml) prior to mount and imaging.

Immunofluorescence

Isolated CM adhered onto coverslips were fixed in 4% formaldehyde and permeabilized with 0.5% Triton X for 10mins at r.t.p, prior to blocking in 5% BSA/PBS at r.t.p for 30 mins. Cells were then incubated with primary antibodies diluted in 3% BSA/PBS overnight at 4° C. Primary antibodies used include TNNT2 (1:100, ab8295, Abcam), DAB2 (1:200, sc-13982, Santa Cruz), CC3 (1:300, #9661, Cell Signalling). Cells were washed thrice in 1×PBS, incubated in appropriate fluorescent secondary antibodies Donkey anti Rat Alexa Fluo 488, Donkey anti Goat Alexa Fluo 488 or Rabbit anti Mouse Alexa Fluo 568 and DAPI (5ng/ml) for 60 mins at r.t.p in dark. Cells were washed thrice in 1×PBS in dark before being mounted onto slides and imaged on an upright microscope Ni-E (Nikon). SCA1 immunofluorescence was performed using two independent antibodies from different companies SCA1 (1:50, E13 161-7, Abcam), SCA1 (1:100, AF1226, R&D) for technical validation and no Triton-X was used for permeabilization to preserve cell surface epitopes of Sca-1.

pH3/EdU Imaging and Analysis

For phospho-histone H3 (pH3) immunofluorescence, cells were first permeabilized with 0.5% Triton X in PBST at r.t.p for 10 mins before blocking in 5% BSA/PBST at r.t.p for 30 mins with the rest of procedure as described above using anti-pH3 (Ser10) antibody (1:100, 06-570, Millipore). EdU staining was performed according to manufacturer's instructions (Click-iT EdU Alexa Fluor 488/Fluo 594, Life Technologies). Imaging of isolated adult CM involved 20-40 random fields of view per condition using a 20× objective (Nikon) on an upright microscope Ni-E (Nikon). A total of 8026 adult CMs were imaged and used for quantification across three independent biological replicates of TAC operated mice each for pH3 and EdU. Mice injected with AAV9 constructs at P7 were administered intraperitoneal injections of EdU (Life Technologies, 5 mg/kg) per day between day 9 to day 13.

Immunohistochemistry

For immunohistochemistry, protocol is similar to immunofluorescence described above with inclusion of an antigen retrieval step by incubation with 0.2M Boric Acid (pH7.2) for 1 hr at 55° C. Complete histological sections (4 μm thickness) were imaged using a 10× objective (Nikon) under programmed acquisition to automatically stitch a large 4×4 (P14 mouse) or 6×6 (adult TAC mouse) image together per section. Myocyte quantification on WGA-stained sections was performed using Fiji similar to previously described 56. Watershed algorithm was used to separate closely separated particles and cells with size range from 10 μm2 to 1000 μm2 were included. All quantifications were normalized to area of histological section (mm2).

Knockdown of LINCMs

LNA™ GapmeRs were designed and ordered from Exiqon. Five different oligos were tested per LINCM for knockdown efficiency by qPCR at 48 hrs post transfection and the oligo with the best LINCM knockdown efficiency was used for subsequent experiments. Isolated Sham or TAC adult CMs were transfected with lipofectamine/GapmeR at a concentration of 100 nM and RNA extracted 48 hrs post transfection. Crucially, fetal reprogramming gene (Nppa) was highly upregulated (average ˜27×) in TAC CM compared to Sham CM at the time of RNA harvest, indicating that during the short period in culture, the stress gene response remained intact in the isolated TAC cells. Negative control oligo with no known mRNA, IncRNA, miRNA targets in mouse or humans as well as mock-transfected cells (lipofectamine only) were used as negative controls. At least three to five independent biological replicates were performed for each qPCR experiment. Each experiment had validated knockdown of target LINCM. Sequences of GapmeRs used are as follows.

Gas5 KD:
SEQ ID NO: 63
AGAACTGGAAATAAGA
Sghrt KD:
SEQ ID NO: 64
TTCGGAACTTGAAGGA
Negative control KD:
SEQ ID NO: 65
AACACGTCTATACGC

Real Time qPCR after Knockdown of LINCMs

SuperScript® III First-Strand Synthesis Reverse Transcriptase (Life Technologies) was used to reverse transcribe poly(A) RNA to cDNA. qPCR reactions were performed using SYBR® Green master mix (SensiFAST, Bioline) in a LightCycler® 480 machine (Roche). Threshold cycle (Ct) and melting curve measurements were determined by LightCycler® 480 software. Each qPCR sample had at least three technical replicates on the same qPCR plate. Rplp0 was used as housekeeping gene and Ct values were normalized to mock transfected (no oligo, lipofectamine only) samples. P values from Student's t test and error bars represent s.e.m. At least three to five biological replicates of adult isolated TAC CMs were used for qPCR analysis of each gene. Primers used are listed in Extended Experimental Procedures.

AAV9 Viral Production and Purification

21 bp miR RNAi oligonucleotides targeting Gas5, Sghrt, LacZ or p21 were cloned into AAV9-TNNT2-eGFP-miR RNAi vectors. The target AAV9 vectors were packaged by triple transfection method with helper plasm ids pAdΔF6 and pAAV2/9 (Penn vector core) as previously described 55,56. Sequences of miR RNAi oligonucleotides used are as follows:

Gas5 KD #1:
SEQ ID NO: 55
AGGTATGCAATTTCCTGAGTA
Gas5 KD #2:
SEQ ID NO: 56
CTCTGTGATGGGACATCTTGT
Sghrt KD #1:
SEQ ID NO: 57
GGGTCTTTGCCTGGGTTTGTT
Sghrt KD #2:
SEQ ID NO: 58
TGGAATGTATCTGGCTCAGAA
LacZ KD Control:
SEQ ID NO: 66
GACTACACAAATCAGCGATTT

AAV9-CRISPR Cas9

pCAG-EGxxFP was obtained from Masahito Ikawa (Addgene plasmid #50716). The AAV9-TNNT2-eGFP-miR RNAi vector was modified to replace eGFP with mRuby2 reporter to avoid spectral overlap with the Cas9-eGFP reporter. Two U6 promoters driving expression of sgRNA 1 and sgRNA 2 respectively were cloned into the AAV9-TNNT2-mRuby2 vector. The sequences of the 20 bp sgRNA are listed as follows:

Gas5 sgRNA 1:
SEQ ID NO: 59
GGAGCGAGCGACGTGCCGGA
Gas5 sgRNA 2:
SEQ ID NO: 60
CATGCTGAGTCGTCTTTGTC
Sghrt sgRNA 1:
SEQ ID NO: 61
TTTCGTCTGAGAGTCGGCTG
Sghrt sgRNA 2:
SEQ ID NO: 62
ACCAGGTAGCCACTGACCGT

Results

Single Nuclear RNA-Seq of Left Ventricular CMs In Vivo

Adult CMs are predominantly binucleated and undergo polyploidisation and multi-nucleation during heart failure. To avoid confounding differences in comparing single cells with different number of nuclei, we reasoned that each single CM nucleus represents the simplest unit of transcription. We therefore performed single nuclear RNA-sequencing of PCM1+ CM nuclei isolated from the left ventricles of Transverse Aortic Constriction (TAC) mouse model of heart failure and Sham-operated control mice, as well as human end-stage failing hearts (non-ischaemic dilated cardiomyopathy: DCM) and age- and sex-matched healthy controls. We focused on the left ventricle as it is the major site of pathological initiation of heart failure. PCM1 is an established marker of CM nuclei. Since single cell transcript detection stabilizes at low read depths, we performed RNA-seq to an average depth of 8.5±3.29M mapped reads per sample, for a total of 359 single PCM1+ CM nuclei from both mouse and human left ventricles using a well-published microfluidic single cell transcriptomic platform 20,21,23,24. Correlations showed good agreement of single nuclear expression with matched experimental pooled CM nuclei (r=0.83 Sham, r=0.86 TAC), matched in silico pooled CM nuclei (r=0.94 Sham, r=0.98 TAC), and even with matched bulk left ventricles (r=0.61 Sham, r=0.68 TAC), which contain CM as well as other cell types such as fibroblasts and endothelial cells. In all cases, correlation values plateaued once we had sampled ˜30 nuclei, similar to saturation observed in previous single-cell RNA-seq reports, demonstrating that our chosen sample size had sufficiently exceeded this saturation limit. A recent mouse RNA-seq paper using a similar TAC induced pressure overload mouse model at 8-week post TAC timepoint reported using a cut-off of at least FPKM>=3 (˜1 copy per cell) in at least 1 heart to detect cardiac-relevant genes in bulk mouse heart tissue. In view of potential noise issues in single nuclear RNA-seq, we set a more stringent criteria for genes to be expressed if it had at least FPKM>=4 in at least 5 samples. In total, we achieved ˜4.29 billion mapped reads and identified a total of 4,787 and 7,642 genes expressed in Sham and TAC mouse CM nuclei respectively. Notably, previous whole tissue RNA-seq comparison of TAC versus Sham mouse hearts reported a dramatic increase in the number of differentially expressed genes (1,435 genes) in hearts at the 8-week post-TAC time point compared to 1-week post-TAC, consistent with much more extensive cardiac remodelling at 8-week and similar to the large increase in expressed genes we found at this same time point.

To address any potential issue of technical variability in single nuclear RNA-seq, we performed several controls. First, we undertook technical replicates of the same nuclear RNA-seq samples using three independent library preparations and found good correlation (r=0.99) across all three technical replicates, reflecting a consistent library preparation procedure, and the absence of a batch effect in this regard. Second, we took the same nuclear RNA-seq samples with identical library preparation we had previously sequenced and performed sequencing again and found similarly good correlation (r=0.94). Next, we loaded ERCC spike-in mix at pre-defined concentrations onto two separate microfluidic C1 chips and again recovered good correlation (r=0.99) between single samples within the same chip, and also across two independent C1 chips (r=0.99). Observed FPKM levels for the spike-in mix were consistent at expected concentrations. Taken together, these controls excluded any significant technical variability in our single nuclear RNA-seq procedure.

Core CM Gene Network

First, our single nuclear RNA-seq dataset allowed us to define molecular markers that are present in every healthy CM nucleus. We identified 6 “core genes” that were the most highly expressed in every Sham nucleus, and also in healthy un-operated nuclei, at low coefficient of variation. We recognized that the consistent high expression specifically of Tnnt2, Tpm1 and Myl2, and not other previously assumed markers such as myosin heavy chain genes, imply their ideal suitability as markers for CM identity. Interestingly, the other three core genes were non-coding RNAs, reflecting a previously unappreciated abundance or function of these non-coding RNAs in CM nuclei.

Heterogeneity and Sub-Populations of CMs in Healthy and Failing Hearts

We next explored heterogeneity across samples. Instead of assessing the spectrum of expression level for each gene, we considered each sample categorically as either expressing or not expressing each gene; leading to a “penetrance” value for each gene, defined as the percentage of samples expressing the gene. In general, highly expressed genes were expressed in the vast majority of samples (Spearman ranked correlation r=0.90, p<2.2e-16) but we noted that this observation was more so in TAC than in Sham (FIG. 1C). Consistent with the notion that CM responded to TAC stress by activating a unifying transcriptional program across individual nuclei, we found that among TAC nuclei there was a narrower distribution of correlation values than Sham (p<2.2e-16 Mann Whitney U test). Furthermore, by using either unsupervised hierarchical clustering, principal component analysis or ranked Spearman's correlation, we consistently detected only two distinct large subgroups of nuclei in Sham and TAC respectively, replicated in a further set of biological repeats (FIG. 1E-G).

We performed weighted gene correlation network analysis (WGCNA) for the nuclear subgroups and identified highly correlated gene modules (FIG. 2A-B). Gene Ontology (GO) analysis for the healthy module showed significant enrichment of genes for RNA binding, mRNA processing, RNA splicing, cardiac muscle cell differentiation, cell cycle arrest, cardiac muscle cell development and heart contraction (FIG. 2C). Disease module 1 contained apoptosis and autophagy genes, reflecting well-known pathways in heart failure, and enrichment of genes in regulation of cell motion, transcription factor binding, actin filament based process and actin cytoskeleton organization (FIG. 2D). Disease module 2 was enriched for genes in translation, generation of precursor metabolites, oxidative phosphorylation, response to oxidative stress, cell proliferation and cardiac muscle tissue development, including well-known featal reprogramming markers Nppa and Nppb (FIG. 2E). All three modules also contained important cardiac-expressed genes known to cause human dilated cardiomyopathy, hypertrophic cardiomyopathy and congenital heart disease, reflecting the overall physiological relevance of our gene modules to cardiac function.

Notably, genes in these modules now form a set of novel classifier markers because they are significantly differentially expressed in sub-populations of CM nuclei across Sham and TAC (FIG. 2F,I), otherwise masked by pooled and bulk tissue RNA-seq approaches (FIG. 2G-I). Prominent exceptions to this remain classical fetal reprogramming genes such as Myh7, Nppa and Nppb (FIG. 2H), which were stress-genes readily detectable even at bulk tissue level.

Single Nuclear RNA-Seq of CM from Human Left Ventricles

We extended the same analysis to human CM nuclei from left ventricles of male DCM patients with end-stage heart failure compared with age-matched, male healthy controls. Remarkably, we found similar highly expressed core cardiac genes, nuclear subgroups and reduced heterogeneity in DCM compared to controls (FIG. 9, S3A-F). Gene Ontology analysis for gene modules gave similar functional annotations as mouse. Differential expression of the dedifferentiation marker DSTN was detected at the single nuclear level, but not in bulk tissue RNA-seq (FIG. 9, S3G-H), consistent with reports of increased DSTN protein in human DCM patient biopsies.

Heterogeneous Activation of Cell Cycle Genes in Sub-Populations of CMs During Stress Response In Vivo

Leveraging on the single nuclear resolution to give detailed insight into gene co-expression, we undertook “Quadrant Analysis” (Extended Experimental Procedures) to compare expression profiles of sets of candidate genes, selected based on known importance for relevant CM biology (see methods). We started with “Proliferation” and “Negative regulators of Proliferation” markers in Sham and TAC mouse samples, and found a significant shift of nuclei from Sham in Q3 (Quadrant 3: not expressing either set of markers) to TAC in Q2 (Quadrant 2: co-expressing both sets of markers: 44.4%; p<3.237e-07 Fischer's exact test; FIG. 3A). This suggested that TAC nuclei activated proliferation gene transcription, and the same nuclei concurrently expressed negative regulators of proliferation acting as “molecular brakes” thus preventing successful cytokinesis. Among the candidate markers, Ccnd2 and Ccng1 were the major ones differentially expressed in the subgroup of TAC nuclei. Of note, transgenic overexpression of Ccnd2 induced adult mouse CM to re-enter the cell cycle and proliferate, while overexpression of Ccng1 induced cell cycle arrest by inhibiting cytokinesis and led to multiploidy. Endogenous rate of division of pre-existing adult mouse CM is otherwise very low, with only a small increase during myocardial stress1. Accordingly, Q4 nuclei with high proliferation marker expression alone (6.4%, Q4; FIG. 3A) could be nuclei that retained the uninhibited potential for cytokinesis. Alternatively, there may be negative regulators in Q4 nuclei yet to be identified. Notably, only with the single nuclear resolution could we attain these results because the same population shifts were neither seen in pooled CM nuclei nor bulk left ventricle tissue (FIG. 3B-C).

Heterogeneous Stress-Response of Early Progenitor Markers and Markers of Dedifferentiation

Next, we performed quadrant analysis for co-expression of cardiac progenitors and cardiac transcription factors, and observed upregulation of both markers in a large subset of TAC nuclei (Q2: 42.9%, Fischer's exact test, p=2.548e-05, FIG. 3D). This was again only detectable by single nuclear analysis, and not by pooled or bulk tissue analyses (FIG. 3E-F). Sca1/Ly6a, Kdr, CD34 as well as Hand2, Nkx2-5, Mef2a, Mef2c were the major expressed markers in the subset of TAC CM (FIG. 3J). Endogenous c-Kit derived CMs were previously detected only at the very low percentage of ˜0.03% in mouse hearts in vivo34. Among our samples, c-Kit was detected in only 3 mouse nuclei (0.83% of all nuclei). The cardiac progenitor marker Isl1 was undetected in any sample. In contrast, high expression of Sca1/Ly6a, Kdr, CD34 in failing adult CMs is surprising because these are markers of hematopoietic and endothelial progenitors that only give rise to very few adult CM in vivo35,36. Moreover, Sca1+ cardiac progenitor cells do not express cardiac contractile genes. We therefore assessed whether our Sca1+ nuclei were from progenitor cells or pre-existing adult CMs. In support of the latter, our Sca1+ nuclei co-expressed high abundance of core cardiac genes (Tnnt2, Myl2, Tpm1) (FIG. 3J). Furthermore, Sca1+ nuclei made up a large proportion of TAC nuclei (Q2 and Q4: 81.0%; FIG. 3J) across biological replicates (70.3%), contradicting the alternative possibility that these are progenitor-derived CMs. We confirmed low basal expression of Sca1 RNA and cell-surface SCA1 protein expression in Sham CM and strong upregulation in TAC CMs by single molecule RNA FISH and immunofluorescence (FIG. 3K-N). Notably, we show that Sca1+ CMs co-expressed Tnnt2 RNA and protein (FIG. 3K-N), confirming their identity as adult CMs and not fibroblasts or endothelial cells.

We further hypothesized that stressed nuclei exhibiting the featal gene response would co-express dedifferentiation markers. Indeed, while TAC nuclei clearly had high expression of featal genes, high co-expression with dedifferentiation markers was again only revealed by single nuclear analysis (FIG. 3G-I). Overall, key to the heterogeneous spectrum of stress-response was that upregulated co-expression of progenitor markers (Sca1, Kdr), dedifferentiation markers (Dstn, Msn, Actn2) and cell-cycle genes (Ccnd2, Ccng1) were limited to the subset of TAC nuclei in Q2 and Q4 (FIG. 3J). This finding is important because it suggests that transcription of dedifferentiation and cell-cycle entry genes during stress response in vivo could be controlled by common regulating factor(s) within each of these nuclei.

Novel Long Intergenic Noncoding RNA (lincRNA) in Nuclei of CMs In effort to identify novel gene regulators in our nuclear RNA-seq datasets, we discovered a large number of lincRNAs in nucleus of CMs (LINCMs). Some of these were highly co-expressed with genes within our healthy and disease modules (FIG. 2C-F), raising the possibility that some LINCMs could play a regulatory role for coordinating the stress-response within gene modules. To ensure reliable annotation of LINCMs, we used Coding Potential Assessment Tool (CPAT) to rule out transcripts with coding potential. This led to a curated list of 464 LINCMs, of which 30.4% (141/464) were novel and 69.6% (323/464) were previously reported in public databases (ENSEMBL, NONCODE) or independent published cardiac transcriptome datasets (FIG. 4A). We reasoned that we have detected more lincRNA because our RNA-seq was performed on nuclei instead of whole cells. Indeed, 40.3% (187/464) of our LINCMs were specifically detected only in our nuclear RNA-seq and not in matched bulk left ventricle RNA-seq (FIG. 4B). To ensure a fair comparison between the single nuclear and bulk tissue RNA-seq, we used either similar sequencing depths or ˜8 fold higher sequencing depths in the bulk tissue, and the conclusion was the same: that our novel LINCMs were detectable only via the nuclear approach, and not in bulk tissue. It is hence possible that bulk tissue RNA-seq reads are predominantly occupied by the large pool of cytoplasmic mRNA, diluting out more lowly expressed lincRNAs that are specifically nuclear retained, and which are therefore not readily detected in bulk RNA-seq. Indeed as an example, we found that LINCM6 is barely detectable in bulk left ventricle by RT-PCR but have high abundance in our single nuclear RNA-seq, and confirmed to be nuclear localized by RNA FISH (FIG. 4H-H′).

We explored the possibility of interactions between transcription factors and our list of LINCMs by performing motif analysis of empirical H3K27Ac ChIP-seq peaks demarcating active enhancer chromatin regions43 proximal to LINCMs loci. There was significant enrichment of cardiac transcription factor co-occupancy motifs at these loci (FIG. 4C). Notably, MEF2, a central transcription factor for cardiac development and myocardial stress-response43 was enriched in 57.1% of loci. To provide functional annotation of LINCM loci, Genomic Regions Enrichment of Annotations Tool (GREAT) analysis showed significant specific enrichment of cardiac expression and functions (FIG. 4C). Global correlation of expression levels between LINCM with nearby genes, including cardiac protein coding genes, strengthened with increasing linear chromosomal distance from LINCM loci (FIG. 10, S5B), implying that LINCMs may act through distal regulatory interactions or long-range chromosomal looping interactions. Taken together, this suggests our LINCMs are biologically relevant to CM and could serve important epigenetic regulatory functions.

To ensure that our LINCMs exist in CMs and are not simply sequencing artifacts, we validated 12 candidate LINCMs by RT-PCR (FIG. 10, S5A) and single molecule RNA FISH in isolated adult CM (FIG. 4D-S′), concurrently demonstrating their sub-cellular localization patterns. Intronic and exonic probes co-localized at bright foci within the nucleus (FIG. 4D, asterisk), representing sites of active transcription. Positive controls included highly abundant core cardiac genes Tpm1, Tnnt2, Myl2 and Malat1 (FIG. 4N-Q′) and negative controls included no-probe control (NPC) and sense probe controls (FIG. 4R-S′). We confirmed that LINCM3 (also called Gas5) and LINCM9 (previously annotated 1810058i24Rik, which we now call “Singheart”, Sghrt) were upregulated in TAC CMs, while LINCM5 was downregulated in TAC CMs as compared to Sham CMs (FIG. 4T-Y). Gas5 is located in the nucleus of Sham CMs (FIG. 4T) but is upregulated under TAC stress and co-localized with Nppa transcripts in the perinuclear regions of TAC CMs (FIG. 4U). Sghrt has low basal expression in nuclei and cytoplasm of Sham CMs (FIG. 4V) but is upregulated under TAC stress (FIG. 4W). Gas5 and Sghrt specifically occupied highly inter-connected nodal hubs within the Disease module 2 (Eigengene based connectivity kME 0.87, 0.67 respectively; FIG. 2E), suggesting their potential role as key regulators of other genes in the gene regulatory network.

Gas5 and Sghrt Regulate Cell Cycle Re-Entry of Adult CMs

Our discovery of Gas5 and Sghrt in key nodal hubs presented the testable hypothesis that they regulate co-expressed genes within the same gene network including cell cycle genes: Ccng1 and Ccnd2 and others: Nppa and Dstn (FIG. 2E). To functionally test this hypothesis, we performed knockdown (KD) of Gas5 or Sghrt separately on isolated adult mouse CM (TAC-operated mice) using antisense LNA based GapmeRs and extracted RNA 48 hr post KD (FIG. 5A). To ensure that reliable knockdown was achieved, we performed qPCR and validated that Gas5 and Sghrt were significantly reduced by 67.3% and 86.0% respectively (FIG. 5B-C; Gas5 expression level after KD: 32.7%±8.29% s.e.m; Sghrt expression level after KD: 14.0%±3.50%; s.e.m). For negative controls, we used both non-targeting negative control oligo as well as mock transfected control.

Knockdown of Gas5 in adult TAC CMs significantly suppressed the expression of Nppa, Dstn, Ccng1 and Ccnd2 (FIG. 5D-G). Prior evidence show that Gas5 accumulates upon growth arrest48, is expressed in many tissues including the heart49, and regulates apoptosis50 and proliferation51 in cancer cells. Sghrt, on the other hand, is a novel lincRNA with no previously described functions. Knockdown of Sghrt caused a significant increase in Ccng1, reduction in Ccnd2, but no significant change in Nppa or Dstn expression (FIG. 5D-G).

We proceeded to test how Gas5 or Sghrt regulates adult CM cell cycle re-entry especially during the TAC stress-response. To this end, we again performed Gas5 or Sghrt knockdown independently on isolated TAC CMs and immunostained for pH3 (phosphorylated H3 Ser-10) to check for changes in M phase entry. Both knockdown of Gas5 or Sghrt resulted in increased M phase re-entry, with significant increase of 3.6 fold or 3.7 fold respectively in % pH3+ DAPI+ CM nuclei compared to negative control oligo (FIG. 5H-I). To check how Gas5 or Sghrt affects S phase entry, we labelled cells synthesizing nascent DNA using EdU, and found a significant increase of ˜2.1 fold in % EdU+ DAPI+ CMs after Sghrt KD (FIG. 5J-K). To our surprise, there was no significant change in % EdU+ DAPI+ CMs after Gas5 KD (FIG. 5K), suggesting a dichotomous effect by which the 2 LINCMs may control CM cell cycle re-entry. Furthermore, DAB2 has been used as a marker of CM dedifferentiation and recently reported to be a cardiac developmental regulator. We found a significant increase of 1.78 fold or 1.69 fold respectively in % DAB2+ CMs after Gas5 KD or Sghrt KD (FIG. 5L-M), reflecting that they regulate dedifferentiation of CMs as well.

Gas5 and Sghrt Regulate G1/S and G2/M Cell Cycle Checkpoints in CMs

To deepen our understanding of how Gas5 and Sghrt function, we profiled the expression levels of key cell-cycle checkpoints regulators of G1/S and G2/M phase. Consistent with the absence of S phase entry, Gas5 KD led to reduced levels of G1/S activators Cdk4, Cdk6, Ccne1 and Ccnd2 (FIG. 5G, N-P). Instead, there was concomitant increase in Nrg1 (FIG. 5Q) and reduction in levels of G2/M inhibitor Ccng1 (FIG. 5F), all together explaining the release of cells from G2/M arrest following Gas5 KD. Adult CMs are arrested at G2/M phase and could be poised for release to re-enter the cell cycle when provided with an appropriate signal such as inhibition of Gas5.

On the other hand, Sghrt KD resulted in increased expression of G1/S activator Cdk6 (FIG. 5O), as well as increased expression of G2/M activators Cdk1 and Cdc25a (FIG. 5R-S), together explaining both increased S phase and M phase entry respectively. There were no significant changes in levels of other G1/S activators (Ccne2, Ccnd1, Cdk2) and G2/M activators (Ccnb1, Cdc25b) after Gas5 KD or Sghrt KD (data not shown). Our data provide cell cycle gene expression evidence that Gas5 and Sghrt regulate CM cell cycle re-entry via transcriptional regulation of specific subsets of downstream cell cycle effectors (FIG. 5T).

Gas5 and Sghrt regulate CMs cell cycle re-entry and proliferation in vivo Mouse CMs exit cell cycle at the end of their proliferation window at approximately the seventh postnatal day (P7)4. Hence we assessed Gas5 and Sghrt transcript levels during the normal mouse heart development across this proliferation time-course window. By qPCR on P1 to P56 isolated mouse hearts, we found that Gas5 expression increases between P7 to P10, and decreases from P10 onwards with age (FIG. 6A). Conversely, Sghrt expression transiently spikes at P7 and increases progressively from P10 onwards with age (FIG. 6B). The end of the proliferation window at P7 therefore coincides with an expression spike in both Gas5 and Sghrt, consistent with their potential role at regulating CM cell cycle exit during this stage of development.

To test if Gas5 or Sghrt regulate CM cell cycle exit in vivo and to validate our in vitro knockdown data, we proceeded to perform in vivo CM-targeted knockdown in P7 mice (FIG. 6C,D) using the AAV9-TNNT2-eGFP RNAi delivery system 55,56. We first screened five to seven miR RNAi oligos per target in vitro and chose the top two oligos for each of Gas5 or Sghrt that yielded the best knockdown efficiency, as verified by co-expressed mCherry-tagged miR RNAi oligos together with full length Gas5 or Sghrt tagged with IRES-eGFP reporter in HEK cells (FIG. 11, S6A). Knockdown efficiency was on average 71.8%±0.03 s.e.m for Gas5 and 87.1%±0.04% s.e.m for Sghrt (FIG. 11, S6B-E).

Following injection of either AAV9-TNNT2-eGFP-Gas5 KD or AAV9-TNNT2-eGFP-Sghrt KD in P7 mice, hearts were harvested at P14 and checked for changes in pH3 (M phase marker) and EdU (S phase marker) (FIG. 6G-J). Negative controls included AAV9-Tnnt2-eGFP-LacZ RNAi that does not target any known mouse transcripts56 and phosphate buffered saline (PBS)-only injections on littermates (N=8 mice Gas5 KD oligo #1, N=7 mice Gas5 KD oligo #2, N=7 mice Sghrt KD oligo #1, N=8 mice Sghrt KD oligo #2, N=5 mice LacZ KD, N=8 mice PBS injected). Despite more limited in vivo knockdown efficiency than in vitro (FIG. 6E-F), we found an extent of phenotype that strongly corroborated our in vitro findings (FIG. 6H, J, N). pH3+ TNNT2+ DAPI+ CM nuclei were significantly increased (M phase entry) after knockdown of either Gas5 or Sghrt in vivo (FIG. 6G-H). EdU+ TNNT2+ DAPI+ CM nuclei were significantly reduced (S phase entry) after knockdown of Gas5, but increased after knockdown of Sghrt (FIG. 6I-J). There was no increase in apoptosis as assessed by cleaved caspase 3 (CC3+ TNNT2+ DAPI+ CM nuclei) (FIG. 6K-L), implying that loss of Gas5 or Sghrt did not induce cell death. Instead an increase in DAB2+ TNNT2+ CMs (FIG. 6M-N) demonstrated again that Gas5 or Sghrt knockdown led to CM dedifferentiation in addition to cell cycle re-entry in vivo.

Since histological sections in the mouse heart contain other cell types besides CMs such as fibroblasts and endothelial cells, we took careful precaution to focus on assessing only TNNT2+ CMs. To further confirm the specificity that EdU+ TNNT2+ DAPI+ and pH3+ TNNT2+ DAPI+ nuclei were from CMs and not other cell types, we also co-stained with the PCM1 CM nuclear membrane marker and confirmed consistent co-localization in confocal z slices (FIG. 60). We stained cellular outline using wheat germ agglutinin (WGA) in histological sections and validated significant increase in CM number (#CM per mm2) after knockdown of Gas5 or Sghrt in vivo (FIG. 6P-Q). Cross sectional areas of CMs were significantly smaller in either Gas5 KD or Sghrt KD, compared to LacZ KD controls (FIG. 6R-S). Similarly, we stained for Aurora B and found significant increase in Aurora B+ TNNT2+ CMs after Gas5 KD or Sghrt KD, consistent with evidence for cytokinesis (FIG. 6T-U). Overall, results support the conclusion that Gas5 and Sghrt regulate cell cycle re-entry and proliferation of CMs in vivo.

Cell Cycle Changes Recapitulated by AAV9-CRISPR Cas9 Mediated Deletion of Gas5 or Sghrt In Vivo.

To ensure that the phenotypes we have observed are not due to off-target effects by RNAi knockdown, we designed pairs of CRISPR sgRNAs that specifically delete the promoter and first exon of either Gas5 or Sghrt (FIG. 7A), screened for their individual cutting efficiency via pCAG-EGxxFP complementation assay58 in vitro (FIG. 12, S8A-C) and injected AAV9-U6-sgRNA1-U6-sgRNA2-TNNT2-mRuby2 into P7 homozygous Rosa26-Cas9-eGFP knockin mice expressing Cas9-eGFP under a constitutive CAG promoter59 (FIG. 7B). We first validated that there was efficient and specific genome editing in mouse hearts in vivo (FIG. 7C) with corresponding reduction in transcripts (FIG. 12, S8D-E) from the resected apex of injected mouse hearts containing CMs and also other cardiac cell types. We also confirmed robust co-expression of AAV9-U6-sgRNA1-U6-sgRNA2-TNNT2-mRuby2 with CAG-Cas9-eGFP throughout the hearts of injected Cas9-eGFP homozygous mice (FIG. 7D). In vivo AAV9-CRISPR Cas9 genome edited PCR fragments were gel extracted, cloned and Sanger sequenced to confirm their identities (FIG. 12, S8F-I). Negative controls were injections of AAV9-TNNT2-mRuby2 without sgRNA into homozygous Cas9-eGFP littermates (FIG. 7C; N=8 mice Gas5 sgRNA; N=10 mice Sghrt sgRNA; N=7 mice mRuby2 controls). We also confirmed the absence of any crossover off-target effects by checking for reciprocal genomic regions (FIG. 7C; FIG. 12, S8G,I).

Notably, a similar effect in S phase (EdU), M phase (pH3), cytokinesis (AuroraB), de-differentiation (DAB2), apoptosis (CC3), proliferation (cell numbers/mm2), cell size (cross sectional area), cell cycle inhibition (p21, CALR) (FIG. 7E-V) was confirmed to be consistent with the earlier AAV9-TNNT2-eGFP-miR RNAi KD in vivo data. This therefore provides evidence that the data obtained from AAV9-RNAi KD is validated via the independent approach of AAV9-CRISPR Cas9 mediated genomic deletions in vivo.

Partial Rescue of Function by Sghrt KD in TAC Model of Heart Failure.

Finally, to investigate if targeted inhibition of LINCMs in vivo can provide any therapeutic benefit in a pathological model of heart failure, we injected high titer (5×1013 viral genomes/kg) of either AAV9-TNNT2-eGFP-Sghrt RNAi or AAV9-TNNT2-eGFP-Gas5 RNAi into mice after 4-week post-TAC surgery, the time point at which mice presented with the evident effect of TAC with cardiac hypertrophy (LV posterior wall dimension) and reduced ejection fraction (EF %) (FIG. 8A-E). We proceeded to monitor their progress via weekly echocardiography. Negative controls were AAV9-TNNT2-eGFP-LacZ RNAi injected into TAC littermates and PBS injected Sham littermates (N=6 mice Gas5 KD oligo #1 TAC, N=6 mice Gas5 KD oligo #2 TAC, N=5 mice Sghrt KD oligo #1 TAC, N=7 mice Sghrt KD oligo #2 TAC, N=9 mice LacZ KD TAC, N=14 mice PBS injected Sham). There was no change in either Gas5 KD or Sghrt KD TAC mice during the first 4 weeks post AAV9 injection (FIG. 8B-C). Instead, it was remarkable that from 5 weeks post injection onwards, a partial and significant rescue of EF % and LV wall thickness became evident in Sghrt KD TAC, but not in Gas5 KD TAC mice (FIG. 8B-E). This rescue in EF % by Sghrt KD correlated well with the level of knockdown of Sghrt transcripts in the hearts of 6 weeks post AAV9 injected TAC mice (FIG. 13, S9B). We harvested mice at 6 weeks post AAV9 injection, and confirmed robust expression of the TNNT2-eGFP reporter even 6 weeks after AAV9 injection (FIG. 8F). Changes in M phase (pH3), cytokinesis (AuroraB), de-differentiation (DAB2), apoptosis (CC3), proliferation (cell numbers/mm2), cell size changes (WGA), cell cycle inhibition (p21, CALR) (FIG. 8G-N) were quantified, and notably consistent with the data from P14 harvested mice after AAV9-TNNT2-eGFP-Sghrt RNAi knockdown in vivo. All together, we show that the targeted inhibition of Sghrt can induce proliferation in vivo and rescue heart function even after the onset of hypertrophy in a heart failure mouse model.

Discussion

Our single nuclear RNA-seq study of CM from failing and non-failing mammalian hearts in vivo reveals heterogeneity of the myocardial stress-gene response for the first time. We uncover gene regulatory networks specific for CM nuclear sub-populations and identify hundreds of LINCMs, many of which occupy key nodal network hubs. In particular, Gas5 and Sghrt were identified as negative regulators of CM cell cycle re-entry and proliferation in vivo. To date, Gas5 and Sghrt are the first lincRNAs reported to regulate CM proliferation.

### REFERENCES

22. Shalek, A. K. et al. Single-cell transcriptomics reveals bimodality in expression and splicing in immune cells. Nature 498, 236-240 (2013).
23. Treutlein, B. et al. Reconstructing lineage hierarchies of the distal lung epithelium using single-cell RNA-seq. Nature 509, 371-375 (2014).
24. Wu, A. R. et al. Quantitative assessment of single-cell RNA-sequencing methods. Nat. Methods (2013). doi:10.1038/nmeth.2694
55. Jiang, J., Wakimoto, H., Seidman, J. G. & Seidman, C. E. Allele-specific silencing of mutant Myh6 transcripts in mice suppresses hypertrophic cardiomyopathy. Science 342, 111-114 (2013).
56. Jiang, J. et al. Cardiac myosin binding protein C regulates postnatal myocyte cytokinesis. Proceedings of the National Academy of Sciences 112, 9046-9051 (2015).
66. Movassagh, M. et al. Distinct epigenomic features in end-stage failing human hearts. Circulation 124, 2411-2422 (2011).
67. Movassagh, M., Vujic, A. & Foo, R. Genome-wide DNA methylation in human heart failure. Epigenomics 3, 103-109 (2011).

1-44. (canceled)

45. At least one inhibitor for inhibiting any one or more of the following:
a) a Sghrt transcript and/or a Gas5 transcript;
b) one or both of Sghrt gene transcription and/or Gas5 gene transcription;
comprising an isolated polynucleotide comprising or consisting of a sequence:
i) that is complementary to any one of Sghrt transcripts and/or any one of Gas5 transcripts or their coding sequences i.e. SEQ ID NOs:1-54 or a part thereof; or
ii) that is complementary to any one of gDNA Sghrt sequence provided in SEQ ID NO:67, or a part thereof and/or gDNA Gas5 sequence provided in SEQ ID NO:68, or a part thereof;
iii) a sequence that shares at least 75% identity with the polynucleotide of i) and/or ii),
optionally wherein said isolated polynucleotide interacts with its complementary sequence to block the function of same.

46. The inhibitor according to claim 45, wherein said isolated polynucleotide interacts with said transcripts of Sghrt and so is complementary to any one of SEQ ID NOs:51-53, or a part thereof and/or interacts with said coding region for said transcripts of Sghrt and so is complementary to SEQ ID NO:54, or a part thereof.

47. The inhibitor according to claim 45, wherein said isolated polynucleotide interacts with said transcripts of Gas5 and so is complementary to any one of SEQ ID NOs:1, 3, 5, 7, 9, 11, 13, 15, 17, 19, 21, 23, 25, 27, 29, 31, 33, 35, 37, 39, 41, 43, 45, 47 and 49, or a part thereof and/or interacts with said coding region for said transcripts of Gas5 and so is complementary to any one of SEQ ID NOs:2, 4, 6, 8, 10, 12, 14, 16, 18, 20, 22, 24, 26, 28, 30, 32, 34, 36, 38, 40, 42, 44, 46, 48 and 50, or a part thereof.

48. The inhibitor according to claim 45, wherein said isolated polynucleotide interacts with said Sghrt or Gas5 gene and so is complementary to any one of the sequences provided in SEQ ID NOs:67-68, respectively, or a part thereof.

49. The inhibitor according to claim 45, wherein said isolated polynucleotide is selected from the group comprising or consisting of: an antisense oligonucleotide; a gapmer; a short interfering RNA; a short hairpin RNA; a peptide, a CRISPR-Cas and CRISPR-Cas9.

50. The inhibitor according to claim 45, wherein said isolated polynucleotide shares at least about 90% percentage sequence identity with the polynucleotide of i) or ii).

51. The inhibitor according to claim 45, wherein said isolated polynucleotide shares at least about 95% percentage sequence identity with the polynucleotide of i) or ii).

52. The inhibitor according to claim 45, wherein said isolated polynucleotide comprises a sequence selected from the group comprising or consisting of:
Sghrt miR RNAi:
(SEQ ID NO: 57)
GGGTCTTTGCCTGGGTTTGTT;
Sghrt miR RNAi:
(SEQ ID NO: 58)
TGGAATGTATCTGGCTCAGAA;
Sghrt sgRNA1:
(SEQ ID NO: 61)
TTTCGTCTGAGAGTCGGCTG;
Sghrt sgRNA2:
(SEQ ID NO: 62)
ACCAGGTAGCCACTGACCGT;
Sghrt KD:
(SEQ ID NO: 64)
TTCGGAACTTGAAGGA;
Gas5 miR RNAi:
(SEQ ID NO: 55)
AGGTATGCAATTTCCTGAGTA;
Gas5 miR RNAi:
(SEQ ID NO: 56)
CTCTGTGATGGGACATCTTGT;
Gas5 sgRNA1:
(SEQ ID NO: 59)
GGAGCGAGCGACGTGCCGGA;
Gas5 sgRNA2:
(SEQ ID NO: 60)
CATGCTGAGTCGTCTTTGTC;
and
Gas5 KD:
(SEQ ID NO: 63)
AGAACTGGAAATAAGA.

53. The inhibitor according to claim 45, wherein the inhibitor comprises a pharmaceutical composition and optionally a suitable carrier, adjuvant, diluent and/or excipient.

54. The inhibitor of claim 45, wherein the inhibitor has been obtained from a host cell transformed with or transfected with or comprising a vector encoding for the isolated polynucleotide comprising or consisting of a sequence:
i) that is complementary to any one of Sghrt transcripts and/or any one of Gas5 transcripts or their coding sequences i.e. SEQ ID NOs:1-54 or a part thereof; or
ii) that is complementary to any one of gDNA Sghrt sequence provided in SEQ ID NO:67, or a part thereof and/or gDNA Gas5 sequence provided in SEQ ID NO:68, or a part thereof;
iii) a sequence that shares at least 75% identity with the polynucleotide of i) and/or ii).

55. The inhibitor according to claim 54, wherein said vector is selected from the group comprising or consisting of: a plasmid; a viral particle; a phage; a baculovirus; a yeast plasmid; a lipid based vehicle; a polymer microsphere, a liposome, and a cell based vehicle; a colloidal gold particle; lipopolysaccharide; polypeptide; polysaccharide; a viral vehicle; an adenovirus; a retrovirus; a lentivirus; an adeno-associated viruses; a herpesvirus; a vaccinia virus; a foamy virus; a cytomegalovirus; a Semliki forest virus; a poxvirus; a pseudorabies virus; an RNA virus vector; a DNA virus vector and a vector derived from a combination of a plasmid and a phage DNA.

56. A method for preventing or treating cardiac disease comprising administering an effective amount of said inhibitor according to claim 45 to an individual to be treated under a cardiac treatment regimen.

57. The method according to claim 56, wherein said individual is a mammal, including a human.

58. The method according to claim 56, wherein said cardiac disease is selected from the group comprising or consisting of: myocardial infarction; heart failure; coronary artery disease; narrowing of the arteries; heart attack; abnormal heart rhythms; arrhythmias; heart failure; heart valve disease; congenital heart disease; heart muscle disease; cardiomyopathy; pericardial disease; aorta disease; marfan syndrome; genetic cardiomyopathy; non-genetic cardiomyopathy; cardiac hypertrophy; pressure overload-induced cardiac dysfunction; and damaged heart tissue.

59. The method according to claim 56, the method further comprising assessing the regenerative or proliferative capacity of the individual's heart tissue before, after or during the cardiac treatment regimen, the assessing step comprising:
determining the presence or amount of Sghrt transcript(s) and/or Gas5 transcript(s) in a cardiac sample of said heart tissue; and
where either one or more of Sghrt transcript(s) and/or Gas5 transcript(s) is present concluding the proliferative capacity of said heart tissue is poor; and
where either one or more of Sghrt transcript(s) and/or Gas5 transcript(s) is absent concluding the proliferative capacity of said heart tissue is good.

60. The method according to claim 59, wherein said determining step involves extracting RNA from the cardiac sample and performing single nuclear RNA-sequencing and then comparing the RNA sequences obtained with any one or more of SEQ ID NOs:1-54 and 67-68 or a part thereof to determine whether any one or more of transcripts Sghrt and/or Gas5 is present.

61. The method according to claim 59, wherein said determining step involves assaying for the functional activity of said transcripts, including use of a competitive binding assay for the transcript target.

62. The method according to claim 56, wherein said preventing or treating cardiac disease comprises rescuing or improving heart function or at least partially rescuing or improving one or more of the following: ejection fraction; left ventricle wall thickness; right ventricle wall thickness; left ventricular wall stress; right ventricular wall stress; ventricular mass; contractile function; cardiac hypertrophy; end diastolic volume; end systolic volume; cardiac output; cardiac index; pulmonary capillary wedge pressure; and pulmonary artery pressure.

63. A method for the proliferation, regeneration or dedifferentiation of a heart cell, the method comprising contacting the heart cell with the inhibitor according to claim 45.

64. The method according to claim 63, wherein the heart cell comprises a cardiomyocyte.


SEQUENCE LISTING (68 sequences)

SEQ ID NO: 1 - DNA, 502 bp, Mus musculus
agcctttcggagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtgctagaatagaagaccagaaaatgaaatggtggagtttgaggctggatagacagtttgaaagttaactggttgcatgcttgttcatttggctggcttgcttgggtacaaataatggtttgaataaagaaaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataacatg

SEQ ID NO: 2 - DNA, 3375 bp, Mus musculus
agcctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatg

SEQ ID NO: 3 - DNA, 346 bp, Mus musculus
ctttcggagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtgctagaatagaagaccagaaaatgaaatggtggagtttgaggctggatagacagtttgaaagggctcctgtgacaagtggacatgcagtgactgcacctttgtttctgaggtgcctggatggaggctcaaatagaagatggtgtcagatatattgtgttaaaattttaccattaaagtgtattataacatgaa

SEQ ID NO: 4 - DNA, 3374 bp, Mus musculus
ctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatgaa

SEQ ID NO: 5 - DNA, 2556 bp, Mus musculus
ctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataacatgaa

SEQ ID NO: 6 - DNA, 3374 bp, Mus musculus; identical to SEQ ID NO: 4

SEQ ID NO: 7 - DNA, 793 bp, Mus musculus
tgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtggagtttgaggctggatagacagtttgaaagttaactggttgcatgcttgttcatttggctggcttgcttgggtacaaataatggtttgaataaagaaaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataacatgaa

SEQ ID NO: 8 - DNA, 3362 bp, Mus musculus
tgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatgaa

SEQ ID NO: 9 - DNA, 1066 bp, Mus musculus
tgagcaggaatggcagtgtggacctctgtgatgggacatcttgtgggatctcacagccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtgctagaatagaagaccagaaaatgaaatggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataacatgaa

SEQ ID NO: 10 - DNA, 3352 bp, Mus musculus
tgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatgaa

SEQ ID NO: 11 - DNA, 1614 bp, Mus musculus
attctgagcaggaatggcagtgtggacctctgtgatgggacatcttgtgggatctcacagccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataacatgaa

SEQ ID NO: 12 - DNA, 3356 bp, Mus musculus
attctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatgaa

SEQ ID NO: 13 - DNA, 557 bp, Mus musculus
gagggcgtgtgaggggcggggtttaagtggacgcagcaagcagctttgcgtcttgacgtcacgaagaccttatatactcgactcagccgccatctcagcctttcggagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtgctagaatagaagaccagaaaatgaaatggtggagtttgaggctggatagacagtttgaaaggtacaaataatggtttgaataaagaaaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataacatg

SEQ ID NO: 14 - DNA, 3471 bp, Mus musculus
gagggcgtgtgaggggcggggtttaagtggacgcagcaagcagctttgcgtcttgacgtcacgaagaccttatatactcgactcagccgccatctcagcctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatg

SEQ ID NO: 15 - DNA, 665 bp, Mus musculus
cccctcagcctgtactcctcagggaggcggagggcgggcctatcgtgcccgcggcaactccgccctccgggctctcgggggcgtggccagagggaaagttttgtgggcctgaagaaggtggggcttgaggaggagtctgagggcgtgtgaggggcggggtttaagtggacgcagcaagcagctttgcgtcttgacgtcacgaagaccttatatactcgactcagccgccatctcagcctttcggagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtgctagaatagaagaccagaaaatgaaatggtggagtttgaggctggatagacagtttgaaaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataaca

SEQ ID NO: 16 - DNA, 3607 bp, Mus musculus
cccctcagcctgtactcctcagggaggcggagggcgggcctatcgtgcccgcggcaactccgccctccgggctctcgggggcgtggccagagggaaagttttgtgggcctgaagaaggtggggcttgaggaggagtctgagggcgtgtgaggggcggggtttaagtggacgcagcaagcagctttgcgtcttgacgtcacgaagaccttatatactcgactcagccgccatctcagcctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataaca

SEQ ID NO: 17 - DNA, 430 bp, Mus musculus
gacagcccggagccaaaggggcggaaggtcgccgagtgctgggaggggaggtggggcagcgactgagcgccgcaggagctggctgcaacccagacgcggcccgggagcgtcgggaatggcagtgtggacctctgtgatgggacatcttgtgggatctcacagccagttctgtggcaaaggaggatgaaggcttacgaggactcgtcaggaagctggataacagagcgagcgcaatgtgctagaatagaagaccagaaaatgaaatggtggagtttgaggctggatagacagtttgaaagggctcctgtgacaagtggacatgcagtgactgcacctttgtttctgaggtgcctggatggaggctcaaatagaagatggtgtcagatatattgtgttaaaattttaccattaaagtgtattataacatgaa

SEQ ID NO: 18 - DNA, 3933 bp, Mus musculus
gacagcccggagccaaaggggcggaaggtcgccgagtgctgggaggggaggtggggcagcgactgagcgccgcaggagctggctgcaacccagacgcggcccgggagcgtcgggtatgagctaacggggcgggggtgcggggaacgagcccagtcaaggagcagcacctacctccgggggtggaaatgggccggaatgggccggaacaccgtcccggaagtgaaaccccccgccccctcctccggagcgagcgacgtgccggaaggaaatcagtcaccctcaccccgccccacccccagcgctttccttgcaggagccccctcccctcagcctgtactcctcagggaggcggagggcgggcctatcgtgcccgcggcaactccgccctccgggctctcgggggcgtggccagagggaaagttttgtgggcctgaagaaggtggggcttgaggaggagtctgagggcgtgtgaggggcggggtttaagtggacgcagcaagcagctttgcgtcttgacgtcacgaagaccttatatactcgactcagccgccatctcagcctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatgaa

SEQ ID NO: 19 - DNA, 712 bp, Mus musculus
gcagagccccgcggcaagggagttgcgggcgcacaggaatggcagtgtggacctctgtgatgggacatcttgtgggatctcacagccagttctgtggcaaaggaggatgaaggcttacgaggactcgtcaggaagctggataacagagcgagcgcaatgtgctagaatagaagaccagaaaatgaaatggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtattaatgggtcacctcaagtgaagg

SEQ ID NO: 20 - DNA, 3110 bp, Mus musculus
gcagagccccgcggcaagggagttgcgggcgcacaggtgaggcgcgggaccgggaagggggcggcgccccgggatcggtctagggcggcggaggttgccggggcgggggtgtgtctgggtgtggccccggcgcgcgcgcggggtcctgagggaggggctcgccaagggggcgggcccgcgagctcgacagcccggagccaaaggggcggaaggtcgccgagtgctgggaggggaggtggggcagcgactgagcgccgcaggagctggctgcaacccagacgcggcccgggagcgtcgggtatgagctaacggggcgggggtgcggggaacgagcccagtcaaggagcagcacctacctccgggggtggaaatgggccggaatgggccggaacaccgtcccggaagtgaaaccccccgccccctcctccggagcgagcgacgtgccggaaggaaatcagtcaccctcaccccgccccacccccagcgctttccttgcaggagccccctcccctcagcctgtactcctcagggaggcggagggcgggcctatcgtgcccgcggcaactccgccctccgggctctcgggggcgtggccagagggaaagttttgtgggcctgaagaaggtggggcttgaggaggagtctgagggcgtgtgaggggcggggtttaagtggacgcagcaagcagctttgcgtcttgacgtcacgaagaccttatatactcgactcagccgccatctcagcctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaagg

SEQ ID NO: 21 - DNA, 600 bp, Mus musculus
agcctttcggagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtgctagaatagaagaccagaaaatgaaatggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtacaaataatggtttgaataaagaaaggtattaatgggtcacctcaagtgaaggcactgcaaacacaat

SEQ ID NO: 22 - DNA, 2384 bp, Mus musculus
agcctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaat

SEQ ID NO: 23 - DNA, 904 bp, Mus musculus
agcctttcggagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtgctagaatagaagaccagaaaatgaaatggtggagtttgaggctggatagacagtttgaaaggtacaaataatggtttgaataaagaaaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataa

SEQ ID NO: 24 - DNA, 3371 bp, Mus musculus
agcctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataa

SEQ ID NO: 25 - DNA, 723 bp, Mus musculus
ctttcggagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtg

SEQ ID NO: 26 - DNA, 1891 bp, Mus musculus
ctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtg

SEQ ID NO: 27 - DNA, 759 bp, Mus musculus
ctttcggagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtggagtttgaggctggatagacagtttgaaaggtacaaataatggtttgaataaagaaaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataac

SEQ ID NO: 28 - DNA, 3369 bp, Mus musculus
ctttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataac

SEQ ID NO: 29 - DNA, 539 bp, Mus musculus
tttcggagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtgctagaatagaagaccagaaaatgaaatggtggagtttgaggctggatagacagtttgaaaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatgattggtcattctgaatttccggtccttcattctgaatttcaaaggctcctgtgacaagtggacat

SEQ ID NO: 30 - DNA, 3080 bp, Mus musculus
tttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacat

SEQ ID NO: 31 - DNA, 872 bp, Mus musculus
tctgagcaggaatggcagtgtggacctctgtgatgggacatcttgtgggatctcacagccagttctgtggcaaaggaggatgaaggcttacgaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtggagtttgaggctggatagacagtttgaaaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaat

SEQ ID NO: 32 - DNA, 3293 bp, Mus musculus
tctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaat

SEQ ID NO: 33 - DNA, 403 bp, Mus musculus
gaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtacaaataatggtttgaataaagaaaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgcctggatggaggctcaaatagaagatggtgtcagatatattgtgttaaaattttaccattaaagtgtattataacatgaa

SEQ ID NO: 34 - DNA, 1845 bp, Mus musculus
gaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatgaa

SEQ ID NO: 35 - DNA, 370 bp, Mus musculus
aagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatgattggtcattctgaatttccggtccttcattctgaatttcaaagtaagtctgtcacttgtttttgtacagattgtaatatagcttgtaaccagtgatgtgatgattctgccaaatgatacaaagtgatatc

SEQ ID NO: 36 - DNA, 817 bp, Mus musculus
aagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatc

SEQ ID NO: 37 - DNA, 422 bp, Mus musculus
gtacaaataatggtttgaataaagaaaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattata

SEQ ID NO: 38 - DNA, 1245 bp, Mus musculus
gtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattata

SEQ ID NO: 39 - DNA, 549 bp, Mus musculus
aggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatgattggtcattctgaatttccggtccttcattctgaatttcaaaggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataaca

SEQ ID NO: 40 - DNA, 1183 bp, Mus musculus
aggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaaatgtgcatgtattagttactttcaaccccttccaaacaagccgataccatgatgataacatagttcagcagacttaactctgatgaacaatcatgtctttcgctcctatctgatgtatctggctgtaatgttttaaagagttttccttgaatagtattaggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataaca

SEQ ID NO: 41 - DNA, 636 bp, Mus musculus
taggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgcctggatggaggctcaaatagaagatggtgtcagatatattgtgttaaaattttaccattaaagtgtattataacatg

SEQ ID NO: 42 - DNA, 820 bp, Mus musculus
taggaaatacaagataagagtcccaagtgatgaggccaaagcctgatgctaaggtggcatgtgctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatg

SEQ ID NO: 43 - DNA, 572 bp, Mus musculus
ctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaaggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatgaa

SEQ ID NO: 44 - DNA, 759 bp, Mus musculus
ctgtgccactgacttaacccagtaggacaattttattgtgaaaaatacataagttccctagggtatgacgattgaaactataaatctcaggcctcttggcaagagaattgatgtgagcccatggagactagtgaagacaactgaacactgagagaatagtgtgaaataaaatgaagataagtaactgcactaattatttttaattttcaaacagattggtcattctgaatttccggtccttcattctgaatttcaaagtaagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttctgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcaggtgcctggatggaggctcaaatagaagatggtgtcagatatattgtgttaaaattttaccattaaagtgtattataacatgaa

SEQ ID NO: 45 - DNA, 530 bp, Mus musculus
gagctgtgcggcattctgagcaggaatggcagtgtggacctctgtgatgggacatcttgtgggatctcacagccagttctgtggcaaaggaggatgaaggcttacgaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttg

SEQ ID NO: 46 - DNA, 1458 bp, Mus musculus
gagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttg

SEQ ID NO: 47 - DNA, 779 bp, Mus musculus
ttcggagctgtgcggcattctgagcaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtggagtttgaggctggatagacagtttgaaaggtacaaataatggtttgaataaagaaaggtattaatgggtcacctcaagtgaaggcactgcaaacacaatggtaagtctttgaa

SEQ ID NO: 48 - DNA, 2393 bp, Mus musculus
ttcggagctgtgcggcattctgagcaggtatgcaatttcctgagtatgttatggcttcgggccttggaggctgaagcactggaagctggtgggattcccggcgcagccggtgagcttagttcagggcacaagctggaaactttaggaggttggttctgcgtgtatttgtgacataaaatagcattgggttttggtctggacaccagacaaagacgactcagcatgcgtgttcatgctatgatgaaggctatgttggtagggacaactgagcttgttgatgaataccaacgattctgatggcagagcatgctgagtcacctcgtgcacggttcaacgggggtggaatgcgaagccacgcggtgtttctttcgcgatcaccatgggatgaaggcttttgctagagattgccagcaggccttagtcactaacaaagaggacgattgctcttgggaagtacctggattagagacttatatgtaactgctggatatgtgcaactcaacagtgcctatgtggacggagttctttgtaagacaggtccattttgtggaagtagaactactaaagattaacatttttcttaggaatggcagtgtggacctctgtgatgggacatcttgtgggatctcacagccaggtaactcagtgggggaggggcatgactagaacataatgaccacaaagcccatgatggtatgagagtagtggacagaagggatttctgaaaaacacttttctgaggctttaattcagctactggaaagggtttagttccgtgaagaaggggactgactgttcacatctcttatttccagttctgtggcaaaggaggatgaaggcttacgaggtgagtactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtacaaagggacatgcagagactttgaatcatcaaaactccagcatgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaatttttttcaggtacaaataatggtttgaataaagaaaggtaagcattgtaagtttggtatttactatatgaattaaggtactgttagtgatgatcaataaagttaaacagatgggaatctctctgaataagattgaagattgattgttaagctgaaacagtatttaaggacatcagtaaatatactttacaaatacatatttctaaacggagtgtaatttcttacaggtattaatgggtcacctcaagtgaaggcactgcaaacacaatggtaagtctttgaa

SEQ ID NO: 49 - DNA, 629 bp, Mus musculus
tactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtggagtttgaggctggatagacagtttgaaaggtaagtattgaaaaacacttgaatttgggtcagtac

SEQ ID NO: 50 - DNA, 869 bp, Mus musculus
tactgaatagtaatgagaagttaggttgaaaggacagtgccacaatgatgacatcatatttgctactcttgacagctggggtgacgatagctttaaaccaccatgattataactgaggcacacaaaataatgatttgggggctacttacagagtaagcagtttttacataaagattccccccccccaggactcgtcaggaagctggataacagagcgagcgcaatgtaagcaattttgtgtgtgcatgtggtcccattacaaggaatacccaatggcaaatgagcactaaagatccctcaacccacacattgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaatagaagaccagaaaatgaaatggtaagtcaaactaaaggaattgtagatgctaaacaggcttggctttgttgcactctgccaaatactggccacgtgttccatcctggtcatagctgaacacagcctgcatgatgacgaacaaatactgactacctgaagatcttattagctctatctgatggtgggcttccttgacatttattgtggggcaaactcaaatctgttgactgcattgaaaagctctttaatgtttttttctaggtggagtttgaggctggatagacagtttgaaaggtaagtattgaaaaacacttgaatttgggtcagtac

SEQ ID NO: 51 - DNA, 2521 bp, Mus musculus
ggggcgtaaaccggaacctggcagcgggttccgccctcgcgccggggaggagtcccggagccgagctggagctggagcacctctcacaccctcctcgccctccccaccgacatcatgctccagttcctgcttggatttactttgggcaacgtggttggaatgtatctggctcagaactatgaaatgccaaacctggctaaaaaacttgaagagattaagaaggacctggaagccaagaagaagccccctagttcctgatgcctgcctggcactgcaatctggacccaccagttccatcctggggggcctgtctccttcacagccacgtccaaagctgtgttcctctttggtctcagaccctgcgcgttgctgaggctccagcacgggccagaagtggagttagctttccgtttccagcatctccccgttcccgtatcttccgtccacctgcttctgagacctaagcctggaaccatggtgctaggttagcagccgcaaccatgacaggactcactcgcctgtgctctgggattcctaccgcctactgtcaagaaatgaatggatttggatagctgctggggactcactgtctagcagtgataacaggagcttgctagacaaactaaactgtgttaaagtcattaaagtcagtatcttcaagccagtttctggtgaattaatccatttattatatattgttaccaaggagctccatggttgatgatttaaccagtaaatcatttgctggtcattaaccaggaaactattttatgcattctataaaaagcaaacaaactgggaaaacttaaaacatgcaaagtaactgttgtcttcgtacagttcagtctatgcagtctatgtttttccttaatgatatatttgaactgaatctgccgtaaaagagccattctttatgctggcacttgataggatgggtggataaatgaggagctgttgataaaataatgttacttttttttttttaaacattaaaatgtatcctaattatataacttcatagtgaaccccctaagagaagtgtggtgcaaatgtgtggtaaagtgagtctctctgggatctgggatctgatgcttcccccacccccgccccccacccccacccccacccaccccccgcccccggttgaggcatcctccccggaagcaggacggaccatcaggcgtcagcaacttcattttttttaaagacagtctcccagtgttacccagggtggtctcaagctcctgggctcaagtaatcctcctgcctcagcttccgggtgctgggaccacaggtgtgcaccatggtgtccagcctagtccgtaatgtttttaattaaaaaatatttaaaaattattcccacaggagatgataggaaagtgatttcacatgaagactctttctagcctctgtcttattcccaggtacaattcctcaggcaatccctggtgtcaggttcctgtgtgcctttgagaaggagtgtctaaccacataagtgttttcgtcttttgttacatacatacatacatacatacatacatacatacatacatacacacacacagtatgtatgtattgtgtgtgtgtgtgtacacatatgctttttgtctcttcctatcacttttgtttttgcacagagtcatgacgcccacgcttttacagtcagtgataacattgaatttctgaccctcctgcctctgcctctcaggtgcagggatcgtaggtgtgcactgccatgcccaactgtgctttttaaagctctttttcttttctttcatttgcttgtttttttgagatagggtagccccagctggcctggaactcactctgtagaccaggctggcttcatactcccagagatccgtctgccctttgcctcctgagtgctgggattgaaggtggctccatcatgcttggcttgcttctttattcttaatggagagcatcccccttccgtcagtacgttaaccttccccctttgtttactgtgcgcggttccactgaatgggtgtttcataattgaatgtgctactgctgagtgcttaggctggtcttctgctggtgtggtgctagcactgaacctgatgcccatatctgcccagagtagagctataggataacgactcacagtctagtttgctaagacacggtgtgtctcgtttcagagggcttattgggcaacttccctgacaatgaatgccttttttgaagaccacaggttgcaaagataagttagggacatctgtctgttttcgggaagtgaggacagtgaccccatatgggaatcagaacatggcattgctccttagagatacattccatccctgtcaaggtcatctctgtatacgagacagaggttggcttctccctagcagtggggagtggggagtggtgtgttcttctgagactctgtaacatctacctcatgagggtttcctgggaaacagtgagatgatgttctgtgctctgttagactgtgctttgcaaactgtccaataaatagtagctacccattggaaaa

SEQ ID NO: 52 - DNA, 1232 bp, Mus musculus
acctctcacaccctcctcgccctccccaccgacatcatgctccagttcctggtgagtggggcctcggaggatcgggcctcgcggtcgcctcgttcccccggggtcccgggtcggcggtgcacggctctgcagttggggctcgtcgccctcgggtttgctcgcgcagttttcctctggcctcctcctaagtccttgggtctttgcctgggtttgtttatatgtagtccttcaagttccgaaaactttggggtgtcagtgatatgactgcgttttcaaaaaaccacttactgtgagctcttggcgaaatcttcgcgcttggatttactttgggcaacgtggttggaatgtatctggctcagaactatgaaatgccaaacctggctaaaaaacttgaagagattaagaaggacctggaagccaagaagaagccccctagttcctgatgcctgcctggcactgcaatctggacccaccagttccatcctggggggcctgtctccttcacagccacgtccaaagctgtgttcctctttggtctcagaccctgcgcgttgctgaggctccagcacgggccagaagtggagttagctttccgtttccagcatctccccgttcccgtatcttccgtccacctgcttctgagacctaagcctggaaccatggtgctaggttagcagccgcaaccatgacaggactcactcgcctgtgctctgggattcctaccgcctactgtcaagaaatgaatggatttggatagctgctggggactcactgtctagcagtgataacaggagcttgctagacaaactaaactgtgttaaagtcattaaagtcagtatcttcaagccagtttctggtgaattaatccatttattatatattgttaccaaggagctccatggttgatgatttaaccagtaaatcatttgctggtcattaaccaggaaactattttatgcattctataaaaagcaaacaaactgggaaaacttaaaacatgcaaagtaactgttgtcttcgtacagttcagtctatgcagtctatgtttttccttaatgatatatttgaactgaatctgccgtaaaagagccattctttatgctggcacttgataggatgggtggataaatgaggagctgttgataaaataatgttacttttttttttttaaacattaaaatgtatcctaattatataacttcatagtgaaccccctaagagaagtgtggtgc

SEQ ID NO: 53 - DNA, 690 bp, Mus musculus
gagctggagctggagcacctctcacaccctcctcgccctccccaccgacatcatgctccagttcctgtccttcaagttccgaaaactttggggtgtcagtgatatgactgcgttttcaaaaaaccacttactgtgagctcttggcgaaatcttcgcgcttggatttactttgggcaacgtggttggaatgtatctggctcagaactatgaaatgccaaacctggctaaaaaacttgaagagattaagaaggacctggaagccaagaagaagccccctagttcctgatgcctgcctggcactgcaatctggacccaccagttccatcctggggggcctgtctccttcacagccacgtccaaagctgtgttcctctttggtctcagaccctgcgcgttgctgaggctccagcacgggccagaagtggagttagctttccgtttccagcatctccccgttcccgtatcttccgtccacctgcttctgagacctaagcctggaaccatggtgctaggttagcagccgcaaccatgacaggactcactcgcctgtgctctgggattcctaccgcctactgtcaagaaatgaatggatttggatagctgctggggactcactgtctagcagtgataacaggagcttgctagacaaactaaactgtgttaaagtcattaaagtcagtatcttcaagc

SEQ ID NO: 54 - DNA, 8922 bp, Mus musculus
gagctggagctggagcacctctcacaccctcctcgccctccccaccgacatcatgctccagttcctggtgagtggggcctcggaggatcgggcctcgcggtcgcctcgttcccccggggtcccgggtcggcggtgcacggctctgcagttggggctcgtcgccctcgggtttgctcgcgcagttttcctctggcctcctcctaagtccttgggtctttgcctgggtttgtttatatgtagtccttcaagttccgaaaactttggggtgtcagtgatatgactgcgttttcaaaaaaccacttactgtgagctcttggcgaaatcttcgcggtaagttgggtcggaggagtcatcaagatgacttggctcctgtcctctccacgcgacttcccgtagacccccatctattatatacggcttatttgaggcctcagagtgcctttcacacgccgtgcattagtcagccacatcacgaggtcgcagcgagagatccacagtgccctccctcttcagacctttacagattgcccgctttgaggagtgagtcagactgccctgcgagtaactcttgtaaaaacctgcccctccccacaccctttcaagtctcctttttgagttaacacgtttttctggcaaaaatcaagtatttctaaaattgcctgttgaggattctaggaaacttgccagtcaaattttcagatgaaagttttcaaacactttcagatgaaagcgtttcatcttcgaattctaataaagcagatatcttctttctttcccacggtcagtggctacctggtgctccgagaatgagtattgtttacataaaactgccctacaaagactcagttgcctctggggtttctctagtgtcctaactaggccttagtttaggaggtaggagtaattagcagtaatttacttgttaggaatcacttttaaaagcagcatcagccagctagtgtaaggtgataggattggcccttccactgcatttgctgttcattgataagcagggagaacattaaatcgattacacttaaccttttaagtgctttgctgaatgaggctacctggtgcgggactagcgctctgtacttaaacatgggttttgattggaaaattcaaaattttcaacatttatggagagatgctgaagccgaccattagatgcttagctaggatgtaactgatgttccttctcttgccctgggtctgtctcctcttcattgcttttgttgggaaaagtggaatcagatgtacagagatggaatcaactttcctcttgtggaacttggtcaaacactgctagtttctgcaaatcttgctttttacattagccgtttcaactgttttctaaatggttaagagtttagaccctatctttaatctcaaggtgtgtgtgtatgtgtgtgtgttggggacgtggggttgggtgggtgttggccaggttatttaaattttctatgcttcagtttgcacctcatgaggtttcaaaaggagccaagatgttgaacacgtaaacactaaatcgtggttgagatatactgccctgtctcttctgtaaggtatcagaaatgggaactggcctcccactgataggagaggcttgggtgtggtggaaagattccaggctcttccctttgatggtgcaaggcagctgaatactttgtaacctctgagatgttgcctaagttgaaaacttgcatttgattgtgtggcttgtctacccattacccctatttccacaccgcactccacagcttgctcctagcttggctgaagttctttgaaggaacacctagatgggaattggactgtgagcactgctgtgagtgtcagggcacctgaacactgctacagtttctgttcctcttccccctggggcccagtagcatcacctcctcataactcaccctcattcagctgctggggcctgtgcaccgtgctgtgtgggttctgtcctgggatagctcagaattgcggagtgaggggaccatatctgggtgtagcagctgggaggctctctccctgtctctaatcttgtctcccgctgcttgcttctgctcttagttcctgtgaagtgcttaagtccggagctggctgtgcttcctaaactcacccttagggaacgggctcctccccaagccagcacctttcacatcagcttccaggacaaccttgaatccccttctctgtgcttccagatcccaagtatcaactgtcagtggctttcctttgcctttctctcagcctctactcatctggattgagtttgccttcctgctttcccagtcctttccccacacttaacgctcacatttgaaatgttgacatagtcattgatttctttctcgtattcatgcattcaccggtttatctagtcttttcttagaatcatgtaataccgtaggtgcatcttctttgttacattgcatccgtttatctgttgatgactgtgcgtgcctctcctgtgagggcctgcacgtgcaagaagtctgaggacagcctccagcagccggttctccttccctcatgtgggtctagggggttgagctcaggttgccaggctcgggtgcagggtgttaccctgctgagccatttttccacgccagacctgctgtgtagtcacgagtaagatctttacttgctccgtgcctcagtttcccccagcataagcctgggcagtaatagtggttattccacgtgtgcatgcttgtgaggatcaggctgtgaactagcagggttctccagtctggctggcatactgccacttctctccactactaaaggatttggttggggatataaagggacttagtcaatacgcagtgagtgacttcgtttgcactcagctctgcatattgctgggcccatccgcatatttaactaaccatggattagataaatatgaatgaatatgaaaaatataaatattagcagatagttgctcatcaatggtatgtgtacagtttttccttgccgttcctgagcagtgcagtgggatagactgccgcaggccttccatacagtaggtcttatgagtgatcaagagggttagagtgtatgggaggacatggggggctcccgtgcagttgtaggacctgcttgaatagctacagatgttggtgtggggggtgtcctgggatggagcccctcattaattttaggtcttccttcttcacctcattactaccgttgattataagatagtggttacaaggtagctccagcttcttttagagcaggcaaacccgtacacctttcccagagagatttcatgtacttctgttgaatattggcacattaaggaggtttttttttctccttcttttcttctctaagcagtattactgaatataaagcatgctttcctaggaagcagtgaagggctttagccctagcaaggttttgctttaatacacagaatgtagggttgtttttttttaaactactaaacatttggtgatagtcttcacactgagtttaaaaactaagacagagaaatggtagtgtgtgtacatgcctccttggaaggccttctgatttcttcttttgaatctgaatttaagaatgctgttgccatggctgaagagatgcctcggctgttgagagcgctggtcgctcttccaaaggacctgggtttgatttgctgtacattgatagaggctcacaacatctgtaactcgagtcctaggggttctgatgttctctttggcctctgtgcatactgcacgcatgtggtgggcattatattatacatgtgggcaggacacccatacaggtaaacataaatagcattttaaaatattttatcaacttagctttttattagttctagttagttaatgtacagttttttgagttgtagatgcttttaccataaaccaaatgtcatttttaggtttccttttcagtgctggcatgagcctatatttgcttaacaatttttaaaaaagattttattattttaagtgtgcttgtgctcagggtagaggtgtgagttctctggtgagttgcgggtaggcatgagctgcctggtatgggtgctaggactgagctcgcatcctctgaaagagtagcatgagctctgagtcgctgagccatctctccaaccctttttaaaaatagattcttaaatccttatcagtaacttgggcctgggtgtgttatttttgaaaatacagtttgatgagtcacattcggacttgtcagccgctttgaggttttgccaggagctgtgccagataaacattgcggttttatttgctggtctccagacacaactttctgaaagggaaggccctgagtggatggcagatgcatctgttttggagtaggcgctgtgccttttagcagagctttttagtgttaatggataaagcattgttgaccaatctgtttgtgtgttaaaagaaataggtacacttacaataatgtagactcctcatgcgcagactcttccatttattggactggttctattttgcacctctggaatttccattctttctaacaaagtccaggttggaagccatgaagttgtctttggtttcttctacttcactccaggtagttgagatgactttaccaaatttggtgatattgtttgtttgcttgtttgtttgcttgtttgttttgttttgtttggcattgagacaaaggttcacccatttattgtatgtaatccagtggcttcagtgtgctcagagatcgtgtaccttctcattttacataaaaaagaaactgtataatatagggagtcatttctcattcccctctccaaactggacaatcaacagttggttttctatcaatgtggaattgctgttttgtattttttttttaatagaaccatacagtatgtggtcttttctgactgcccttgactgcactgtcaaggctcctctactgacagcatggagcagtgagcgcttcatttctcgatatgatgagtatgtgttaatcagctctccagttgtttccgctcttggttgttaggaatgggctaagccaggaatattgaggaggcatttttgtgggggttcatgtccaccttttggtgcatgcccacactggagttgtcacatcctataactgtgcacacagcactctggaaagtggctagatggtttttcagagcggccatgtgttatacaccagtggaagatgaagacctccgccccatgtcttgccagcatttgttgtttgttttgtttggtttggttttgctgcttttgagtaaagccatctaagcggatgtgccgtggttcctcgtggttttgattagcgtttcctcggtgactgatgtcaaacatctcgtgggcttgttgactccgtgtctttggaggatccactcacagcttctgccactttttaaattgaattatgtatctccgtcatttaattgtgaaagaccagattatgatttgcaaataattctattttgtggtttgtcctttctctttgatgctgttatttgaagggcaaacatttttatatttcgtgtgtgtatgtacacgtgactatgcaggactgtttggctgtgcatacagatgtccaggtttagatttgcttttcttgtctgctcctacctgttgaccatctcctgaagcaagcacgctttcctgacttctgggttctccatcattttcctgagtgagtggcacttctcacatgtgctctgcatcatatttaaaatgtcgcgttgtagatttggtagtattctggaacgggtgccgcgtatagaggccatgggttttgtttgtcttctaaaataactgtccccttttctgccccagtgcttggtgacattgacaaaatagttagttaggcagtgtttattttgagacacacattgtaagaattaggaattctgtgtctgagccggaacgtttcagtgttgggctgattcagaggtgaggaagcgcagcggagctggagcgggttgagtggcccacgaaggcacggtctctcggacgaaggcacggtctcgtctctcttgcggggctactgcttctcacggttcctcgtgccggctaggattcacagcatcaaggagttctttccctctccttttaaattttgtctcaaattaaattatctttctggttttcagaaagttgtggtttgaatctactagagtgaaaaaaatgtaattctgattcttattgctaccttagtaatgacttttaaaaacatcagaaaatggttagcagcttgaaactaggtagcttcttttatgtttcttgctaacaaccaaccaatttaaaactgcctttgggtttaaaggttaagtataagacagacccagttgtagttctccttttttgtgttttctcccttcagggtttatttttactatttatggtggtggtggtggtagtggtagttgtagtgtgcactatgttcactggcatgtggatgccggcagagggcagaataaggtgttggttcctgaagctggagtcagttgtgagctgactaacatgggcgatgagaactgaactctggtactctgcagtggtcctaactgctgagccatctctccagtacccccatctgtatccttgcctcacctgcatctcatgtgtgagtgatgggacacttaatgctgttctttgatttctgtcttaacttctctgttgagactgagttagtggcaataccgtttaaacaaaacaatgggaaaaaagggtagatgccaatttgaatttattaagttagaaattagcccacagttacaagctctgaaataaaaagtcactgagggaaatcagaattatcattaaattgcacgtctgtggaactctggcccatttgtgtatatggagtatttgtgtatatggagtattctacattttcaggagaatactcactcagtggggtaaatgtttacagttctctaaaagttgtccatgtcccttttgtcattgagtgggagccacagaccttaagtgtcctgccctttagtccccacccttggcgtggtgctaggctccgtgatgtgtcagagtttcctgttcttctctgcccttgaggatttgagtccccgagtaactggagttgttgtctgctgctgctttcctgaataaccctgggcaaaccgctctagctcttttgaaataaggagagactaaactctgagagcccctttatttttctaagcttttttgattcaaacgggtttttgttgttgttgttttgagaagaagtcttgttttgtggggatgagacttttgctgggtagctgggactctctgtgtagaccaggatggcctctccaggccaagaatgcccctgcctctgaatgctgatggcaaatgtgagccaccatgcccagctcccgtgggtatttgcaagtgaataccttttttatcagctgtgtacaggagtaacagtacctacaacaaacagtaacgagtaatagtaacaaaatcagaacactggcttccagacctttcaaacttgctcaaagccaccgttattctgtcctaatgttggtgcatggtatatatgtatgtgattgtttcatagaagaaagatgtgacttctctgttggatgcagtcctacactgaggacctgggcagtcacctgccgtgcacttcttgggaagatgcagcccgtgtgtctgacatagttgcaggctcctggtgggttttgcctgcagtgtaactctcctgttctgttctttcagcttggatttactttgggcaacgtggttggaatgtatctggctcagaactatgaagtaagtgactacattcatgtctttcttggcttactgtaaccgtttcatagttacatagttacctctctcaaggtgaagtttgcgtgctaacacagtgaggtctgtaaatgtaactccttaattttatagaaagcttttctgttgtttgagaaagtaactgaactcacaaaggcacggtgtccgcacatcatcccaggagaactggatgtgaatttagcgtggcttgtaggaggaagacggtttgggaaagtgaggagtcacatccacctcactgttggtgtatgagggagggaggaggggtggaaaaatgctgtcacagcccaacgagaagcctcgaatgtgttgcagagagacaaaggattacattttcttctgtacccagggacaggaactttgttgctttcttattgcctatagatacacttcttttatctggcacttgagctgaaggtccaagtgtgtatctaaagggagtgctgggcctggctttcagagaggaaatgtaaatctaacactgatagctaatatatatagatacaagccaaggaaacaccactgagtaggtctccagaggaagccaaagccttggcaaagccctttgtcctttaaagaagagctgtgaaagatactggcaagtctttctagtcttcctatctatagctaaattataatgtttctttttttcctctttaaagatgccaaacctggctaaaaaacttgaagagattaagaaggacctggaagccaagaagaagccccctagttcctgatgcctgcctggcactgcaatctggacccaccagttccatcctggggggcctgtctccttcacagccacgtccaaagctgtgttcctctttggtctcagaccctgcgcgttgctgaggctccagcacgggccagaagtggagttagctttccgtttccagcatctccccgttcccgtatcttccgtccacctgcttctgagacctaagcctggaaccatggtgctaggttagcagccgcaaccatgacaggactcactcgcctgtgctctgggattcctaccgcctactgtcaagaaatgaatggatttggatagctgctggggactcactgtctagcagtgataacaggagcttgctagacaaactaaactgtgttaaagtcattaaagtcagtatcttcaagc

SEQ ID NO: 55 - DNA, 21 bp, Artificial sequence
  Oligonucleotide sequence
aggtatgcaatttcctgagta

SEQ ID NO: 56 - DNA, 21 bp, Artificial sequence
  Oligonucleotide sequence
ctctgtgatgggacatcttgt

SEQ ID NO: 57 - DNA, 21 bp, Artificial sequence
  Oligonucleotide sequence
gggtctttgcctgggtttgtt

SEQ ID NO: 58 - DNA, 21 bp, Artificial sequence
  Oligonucleotide sequence
tggaatgtatctggctcagaa

SEQ ID NO: 59 - DNA, 20 bp, Artificial sequence
  Oligonucleotide sequence
ggagcgagcgacgtgccgga

SEQ ID NO: 60 - DNA, 20 bp, Artificial sequence
  Oligonucleotide sequence
catgctgagtcgtctttgtc

SEQ ID NO: 61 - DNA, 20 bp, Artificial sequence
  Oligonucleotide sequence
tttcgtctgagagtcggctg

SEQ ID NO: 62 - DNA, 20 bp, Artificial sequence
  Oligonucleotide sequence
accaggtagccactgaccgt

SEQ ID NO: 63 - DNA, 16 bp, Artificial sequence
  Oligonucleotide sequence
agaactggaaataaga

SEQ ID NO: 64 - DNA, 16 bp, Artificial sequence
  Oligonucleotide sequence
ttcggaacttgaagga

SEQ ID NO: 65 - DNA, 15 bp, Artificial sequence
  Oligonucleotide sequence
aacacgtctatacgc

SEQ ID NO: 66 - DNA, 21 bp, Artificial sequence
  Oligonucleotide sequence
gactacacaaatcagcgattt

SEQ ID NO: 67 - DNA, 34186 bp, Homo sapiens
aaacatcctaaattccttccattgattgaacagaccctctttttggccaaagggactccagaaagaccttaaaactgagttcccagacatgatggtacgggaggtcagacatgcctcgttatatcccctctcctttgcagtttaaatgcaacaactgatcagcattaatgttaaaacacagaccagggggagtaagctctttcctttctgtctggcagcagccatcaggtaagccaaaatggatgcatacaagtacatccaggagcaatggaggaagaagcagtctgatgtcatgcgctttcttctgagggtctgctgctggcagtactgccagctatctgctgtccacggggctccctgccccacctggcctgataaagcacgccagctgggctacaaggccaagcaaggttacattatatataggattcatgttcaccatggtggtcaaaaatgcccaattcctaagggtgcaacttatggcaagcctgtccatcatggtgttaaccagctgaagtttgctcgaagccttcagtccattgcagaggagcgagttggacacaactgtggggctctaagagtcctgaattcttactgggttggtgaagattccacatacagattttttgaggttatcctcattgatccattctatgaagttatcagaagaaatcctgacacccagtcgatcaccaaaccagtccacaagcagagggagatgtgtgggctgacatctgcaggccaaaggagctgtggccttggaaagtgccataagttccaccacactattgatggttcttgctgggcagcttggagaaggcgtgatactctccagctccaccgttaccactaatataagtaaagtttgggccaggcgtggtggctcacgcctgtaatcccagcactttgggaggccgaggcgggcggatcacaaggtcaggagatcgagaccatcctggctaacacgttgaaacctcgtctctactaaaaatacagaaaattagccgggcgtggtggcgggcgcctgtagtcccagctactcgggaggctgaggcaggagaatggcgtgaacccgggaggcggagcttgcagtgagccgagactgcgccactgcactccagcctgggtgacagagcgagactccgtctcaaaaaaaaaaaaaagtttgtaaaattcgtacctaataatttaggacagtcatgtctgcttacaggtgttatttgtctgttaaaactagtctgcagattgtttcatgaatgctttgtcaaattatgaaaattaaagtgcaataatttttgaagacaataagggatggtgtatcttgtttctaataagatgaactttttttgtctttgctttatcttattagggagttatatatcttattagggagttagtgtttaaaacatactgtgtggtataataggcttaaaataaattctttaaaagagaaagagagaaaaaaacacacacatacacagaccggaagactgacagaacagactctttgtggcgatgatacaaattgtcaacaggacctatggccatcccaggcaagaaacccagctaggccagtgaaactgagagccaacctgatgaggcctgccaaatgcaacttctctcacttgtttttagtcacctgcttttagtggattaaaaaacccacatagctaaagtcacagagctaaacaatatatatctaagctcccactatcttccttatggataacatctctgacatctgggttgcttgataaaggtttcttgtttttcaggaagttggggtcagttcttgtacagtttaaaccaccaacctctcaactgggcctgcatgaaagcccgatgggtgtccttctgacattagagggccaaaacctccacactcagatcatgctaaggatgtcattttgtgaccatgtatcctataaagagccatgaagcttgactacctcgcgcagataacccattgccaaacttttccttaccttcaaccgcctttccccacacctcagaccaccctgctcctctatcccataaatatccctaaaacccatctttggggaggcagatttgagatctgttctcccgtctcctcggctggctgcctggtgaataaaccttttctctgctacaaaactcatcatctcagtgactggcgtgcggcacacggcagcatgggccccatacggtacccagcctctaccttgattacacaacacctgattacacaacgctgattacacagcacacctgcggagctttgagagctacaagcctcctttcctccccgcaaagtccagctgggcccaagtagaggatgctgcaaaagcccccacgattcagctgcgaacctagggatgggacccgctgctcatgccgtccttctaggttagagagccctaaactgggctgtgtatcagaatagtccgcgtgctgagaataatatagattcctgggcctcggcccaggtttacagaggcgggatgctcaaggatgcccagccaggctcgggacttgctcttcgcaagtcctgtcgcgcgctcgctgccccgccggttcaggcggtgaggccaacgcgctccgccagggggaacctgggcgctgcgacactgcaagtcgtttccgggcgcgggctgagtgggaggagcggggcggggcgtggcgtgacccggaagtcgacgaggggttccgccccccccgcgcatggggaggtaggctcggaccggcccgcggagctgctgcagtccttcgcgccctcctcgccctccccaccgacatcatgctccagttcctggtgagtggagctgccgaagagcggggcccgctgcctggggcgtgcctcggaccccatttcccccttgaggattgccgaccccgccgacccggcctgggcgtgggtccagcggtcccttcgtcccccgcgcctggtcgtcgcctcgcagggcggccgttttcttcctgcctccccagaaaatccctttgccaatatttttctgcagccatccaagtccctaaaactttgaggtgtcagagagtgtgcgtttccagagcttcttattgtgtgccgcgccccgcgctcattgtgcaatctttgtattaaagcttgtgtggtccttgatgacttttcgtagtccgcccccctttctttctgcgctcctgttccattacatggtcgccggccggtccctgctgtggagccctgctagttggcttttctgaggaggtaggaaggccaggaataaaccgtaaattagccacctcacgaagtccgagaggctaagtcctcatcagacctttcagagttggaggaatgagtcagacactggtatacaagtaatttttcccaaaatgtgtaagcatgagtaagcagactatttctcttggctccttatttgagttaaaaacgagttttctggcaaaactcggtatatttcaaaaattgcttggtgaaggttccaggagttgctcatcaaattttcaagtgaaagtttttctgccaagctattgtgcattcaaaacttgatgctttcaacctctatttttttttattttttattttttttgagacggagtctcgctgtgtcgtcaggctggagtgcagtggcgcaatctgggctcactggaacctccgcctcccgggttcaaccgattctccctcctcggcctccggagtagctgggattacaggcgcgcaccaggatgctcagctaatttttgtatttttagtagagacgggtttcaccaagttggccaggctggtctcgaactcctgacctcaagtgatcaacccacctcagcctacgaaagtgctgggattacaggcgtgagccactgctcccggctcaaatgctgtatctttttttgttgtttgttttaacggagtcccgctctgtcgccaggctggagtgcagtagtgcgatctcggctcactgcagcctccgcttcctgggtttaagcgattctcctgcctcagcctcccgagtagctggtgcgcgccagcaggcccagttaatttttgtatttttagtagagacggggtttcaccccgttggccgggatggtctcgatctcctgacctcgggatctgcctgcctcagcctcccaaagtgctgggattacaggcgtgagcctccgcgcccggccccaaatgctgtatctttatcaaagagattaaaacacaaaatgcaccccttttgtccttttgaaagatggtaatggattgttaaatccaattagtcaaaagagtgtccttaatctgcccagctaagacagggctgctcctttgcctctaggagtttctgtagtgtcctaactagatgttagtttaggagttcagaataatgacagtcattttcttgtagtaaacactagattaaaagcggtatcacttagctccagagtatagaatgatgtaattgagctgcttactatatctgttgctcattgataaactgagaggacatgattcaattacagttaactttctaagtggttttgcagaatgagtctatttggttttgcaataaacttttactgtgtgttaattttttttttttttttttttttttgagacaaggtttctctttgtcacctaggctagagtccagtggcacaatcatggctcagtgcagtctttaattcctggcctcaagcagccttcccacctcagcctctttagtagtagctaggactacaggcccgcactaccacccgtgactaattttaaaattttttttttgtagagacaggtcttgcctaggcctcccacagtgttgggattacaggcatgagccaccactgtgcctggtcagattctgatttgagaattcagaattctcagtatttaaggagagatgctaaagctgtcaattatattcctagctcagaagtaactgaacactgatgttctctcaccctctggtcttccacctcattgcttttttttttaaataagtggatagcattacttttcacattgattccacctctgtcaaatgagccactgactctaaagttgagcaagtatttctctttgtgtgcagtcatttcatctattaaaaaatgaccatcatgttctagtgtttaagagattaaacttcagagtcagacttctcagtttgaactctgcctcccatcattttcaagctgtggtagcgtgggtgggctattgaaattttctgtgctcccatttccacatctgaaaatgcaaacccacctcacagttttgttgtgaggattaaaaatgagttaacacattaaagaacttaaaaacaggagcactcaacaaggattcagatagtgttcagtttttcttttgcagggttcatttgaaccctgattgagtttcttctcaagtggggattagcttctctcagaagggtaggtgataggaaaggtttagaagtgatgaaacagtcacaagttaagcagtgcgttgtcattacacattttggaaccgtctaagatgtttctcctacagttgtgatttgtggacagtttgaaaaccactgcacttgcttgtgttggtgataacgtccttccattattcatattcctcctcagattaaaatattctccctttgtccctatcttgaagagaccttgttcctgtgattttccccttttcttttactgcagtttttcccctttattattactatttttaatagtggagatagggtctcactatattgcccaggctggtcttgaactcccaggctgaagcaatcctcccacctcagtctctcaaagtgttggattatgggcttgagccactgctcctagacttttttttttttttttaaatgcagtgcttcagaaatttgtgtgtcgtctttgcgcaggggccattctgatctctgtatcattccagttttagtgtatgtgctgccaaagtgagcacccccctttcttttactgcagtaaaagacacctctttgcagatagttaccaacactacctgagggaagctactgcttccttaatcgtaaggtttagctagagttattttaaggatgcttttggcgggaagctaacatgatggcactgctgggggctttgtgcacactgcctatgatgttgttattgtgttattgtaatctgttgctttttttttttttgagacagaatctcactctgtcacccaggctggagtgcactggtgccatcttggctcactgcaccctccgcctcctgggttcaagcgattctcctgcctcagcctccgggttcaagcgattctcctgccttagcctccgaagtagctgggattacaggcatatgccaccacacctggctaattttatattttttagtagagatggggtttcaccacgttggccagcctggtcttgaactcctgacctcaagtgatttacccacctcggtctcccaaagtgctgggattacaggcgtgagctcctgcacccggccggttttccctttatcctttttgccttcctaaatactcttcaaggccccactttgatctttttccatgaggccctcctgatgacttatgcttctgaaacctttacactattatgtctttgttttgttttgtttttgagacagcgtctcactctgtcacccaggctggaatgctgtggtgcaatctcggctcactgcaacctctgcctccttggttcgagcgattctcgtgccttaccttcccaagtagctgggattacagacgtgtgccaccatccccggctaatttttgtatttttagtagaaacagccatgttggccaggctggtctcaaactcctggcctcatgtgatctgcccgcctctgcctcccaaagtgctgggattacaggtgtgagccactgtgcctggccttacactattacactattatgatccatttcctcatttatttaggtctcatgtcttttcagtaacttctgaataattatcttgcaccttgtttgttatgtttatttctattttgttaattatttcaccagttgatggacattcaagatgtttccacttttggctatgatgagtaatcctgctaggaatatttgtatacaggcttttgtgtggatatgtatttcagttctgttgagtatataccctaaagtgggattgctgcattatgtggtatctttatgctcaactttttgaggaacttttggactgttttccagagtagctgcaccgttttacattctcactagtaatgtatgacggcttcaatttctccacatcctcgccaacatttcgtgtagctcttattgtttttgttaaagtaattctagtgggtgtgatgtagttcctcattatggttttgctttgcatttcctcagtgactaatgttgagcatcttttcatatgcttgttgactgtttgtagctttggagaaatcctttcacatcatttgtccatttttatttttgttttattttatttatttatttttgagacggagtctcgctttgtcgcccaggctggagtgcagtggtgcagtctcggctcactgcaacctctgcttcctgggttcaagtgatttctcctgcctcagcctcccgagtagctgggactacaggcgtccaccaccatgcctggctaatttttgtatttttagtagaggcggggtttcaccaagttggccaggctggtaattgaactcctgacctcaggtgatcccccaaccttggcctcccaaactgtgggattacaggtgtgaaccactgtgcctggccaatttgcccatttttaaattgagtttttttgtgtcactcagttgtaagaattatttatatattctggatacacatcccttatcagatatatatttgaaaatattttatctcattctgtgtgttgtcttttgactttcttgatggtgttctctgaaacacagatatttttaattttgatgaggtccaatttttttttttggttacttgtacttttggtgtcatatctaagaaaactatttgcccagccccactcatgtgaaacacagatatttttaattttgatgaagtccaatttttttttttggttacttgtacttttggtgtcatatctaagaaaactatttgcccagccccactcatgaaggtttacgcctatgatttctttgaagagttttatagttttagctcttacatttaggtctttaatccattttgagttaatttttgtatatagtgtgagataggagtccacctttattctttgcatgtggatatcccattgtcccagcaccatttcttgaaaagattattcccagcaccatttcttggaaagactattctttctatattgagttccttggcactcttgttgaaaatcacttgaccataagcctttatggtgtattcctgggctctccactttattccattgatctttattgatcaatagaaacaattcagtcttctttccttgccaccacagtaatcggcttttctctaatgggtacttggttttcttaaaaaaaaaattacctttttaaattcctacatcttttcatcatctaataggtccttgtttcctcagccttttctgagtataggtaccgggagagtgcttctcaggcagtgctttcatcatgttgctgagggcagagtagtcagtgtctccctaccatgattgcatgtgaaggaaactcccctgcctctgatgcccaggctggagtgcagtggcatgatctcagctcactgtaacctctacttcctgggctcaagtgatcctcccacctcagcctccagagtagctgggactacagatgtatgccaccacatccggctagttttgcattttgttggtagagatgtagtttcaccatgatgcccaggctggtctcaaactcttgggctcaggccatcctctcaccttgtcttcccaaagtactgggattacaagcgtagccattgtacctggctgccctacctgcttgttgaagcccaacatctagacctttccagctttccatacttaattctctccattctttaactcaaattctcttttccagtcaggcctgtttcttttatgccagtcatcacatgtccattcccatctcgtgccttgattttccacattcagttttcttcgcatttcctttaccagttgtattagtccattttcacactgctgataaagacatacccgagactgggtaatttacatgggaaaaagggtttaatggacttacagttccacatggctggggaggcctcaatcatggcagaaagcaaggaggagcaagtaacgtcttacatggatggcagcagacaaagagagagcttgtgcaggggaactcctctttataaaaccatcagatctcatgagacatattcactatcacgagaacagcatgggaaagacctgccctcatgattcagttacctcccacttggtccctcccacaacgtgggaattcaagatgagatttgggtgggaacacagccaaaccgtatcaccagtttaaatgtgaacacttagcatatttagagcctttaatatcaacctgaggtatatatttttcaagtatgatttcttaaaagaatcacttttgtgtcattcattttgccacttaatgcatggcctagttctgatgtttggagcatctgttaacattgaaaatcctgtgcagggtcctgccttctgttgttatataactattttacctagcatttcttcctattcatcatataagttctctaagggcagagatcatttttaatcctttcatggggctaaatatgaggttgatttttagtataaggtctgcagtcccttaatcaaaatctttaggggccgtatgtatttgggaaatcagatttctttcagattttagaaaagtaataagctgcatcatgtacctcggtacttaacacccccagcagcatctgggatagcactttgtaatctaaatgtattaaaatttttggtgtgagacatattctccagcgaggaggtctaaggcaggatcctatatttaaagatttctatttctatagcaaaaaatgattattcacactaaagtgtgataatgacataaataggctcctgttagttcaggtcagatatgcttttgctaccaaatgagttatgatgaaatgtagtttggattgagttgggtttcagaattgtggataatggattgtggagcttcactgattcttagtgtactctctatttatagagctgacaatgcgattcttctgttgtgtactgctctgtgccagtctagacttctcttttggggctcttagctccagtatcttcttcccatctatttttcctgtacactactttcctcatctcagtcctctgttctcaaacttccagtgttttaccattaattgtccccaatgtttctgtgaaaagaagctactttttcatacccctaaagtctcaaaattattacagagagggtgtgatagtaatgcacttagtgttgagcttgggaagaaagcattgcattccttatgccacagtcttctctacagggaatactaaggggtgaccatgtgatatgggggaagcagggcattgtgatggcattcagctgacagaactactgatgaggtttccaggtcttggcctggaagaagttaggcattcttggcctgcaaggaggtttcagactcatccaagacctcctataatctgactccagtctaacctctcagactgagtcactgttcctcagatttaggttttacttttttgtctaaacttgcctactgaccatcccgtgaatgacatactgtatatgcttttctggcttcatgcatttttcccaacatttctcttagtgatatttttcttgtgtatttaatcttatgatactcctttggtagatccggtagtattctgaaactggaattgatatgacttgtttagaggctcatggtttttgttttgtgttgtgttttaaaataatcatgagttttttatgtgccctgatacctggcaaagttgccaaaatgaagccacgttagacatgccacatttatttagagacacatttggtaagaactaggaatcttagcatgttggagctggacattacattgtgagtataatgttttataaatcaggaagctcaaatcagcaaagctccagagaggttaagtgtcttgctttaggcacaccgcttgatagtggtggaaccagaactcgattattttttttgaaagtttttaaatcccatgttttttctttttggtgttagagctaatactttcatttgacttgttttctcttcttttaagcaaatttaatactccttctggatttggaaaaccatttcctacctggtttcaacccataggagaaaaaaatcattctccttcaaggtgcaacactggtaaaagtaggattttgcaggccatatctgattaatcaaatgatcagaacggtctatgattagagataagatagactcttctatgatttttattgctaacaacaaaccaatttaaattatacttttggtgtttcaagcttagtaagtttaaagtgtactaaggacccagctgcattctgttcccctgtgtgcatctccaacttgaagagaaggttcagtcatgccatgtgattgatggtgttaagtgccacattttatttttttaaacctctctgctgtgtattgattgaataaaagcaacaatgattaaagaaaatgatgaaaaaaattgatgctagtttgaatttataaaatgagaaattaagctccagatggagcctcttagttaccagaggaaaacagggaattattatcattaaaactcaaagctgaggagccttggcccttttggatatatctaaattttcatgaaccagtttttgtttaggaaaatagtcaccaaatgagataaatatttaaagttttctaaaaattgcacccgtctcttgtcactgtgtttcccaccggccttaaatgtcctgcctgttagactgtacctgtggcagagtgctaagccatatgtgctctcagtggtgtgtcagagttttctcttcttttctgtaatcacatattttagtttttgagtgtgaagctaatgtagattgtcctgtcctccttgttgtgaatgtccttggagaagtcccttcccatcctgggcctcaagttcctcttttgaactatgaagagattgtaaactgtgaagagtcccttcattctctgagatcctatgatttgactgaatatttatttatttttattttgagacagtgtcttgctctgtctcccaggctggagtgcagtggcacaatctcagctcactgtaacctctgcctcccggtttcaagtgattctcctgcctcagcctcccgaatagctgggattacagatgtgagccaccacgcctggctaatttttgtatttttagtagagacgaggtttcaccatgttggccaggctggtctgaaactcctgacctcaagtgatctaccactttggcctcccaaagtgctgggatataggcgtgagcctctgtgcccggccttgactgaatactttaaaaactgaataatttttatttgatttaaaaaggaataacaatatccataaccaaaaaaagacaaaaaaattgcagcatgcaacaagaaaaatctgaactttggcttgagtttccaggacttgtagtcaagtatttatgtgggttgtgaggggataagagaatttagggtagctttatacagtgttaccatttattcttttttgttgttttatttttttcttatgtttggttggtgtcaccatttattcttttgtatgctgtttagcctctggagagggcatctctgtgtttatagaagaaaaatgtgactctcatgttgtacacagccctagactggacatcaaggtcttctaactgtcctaagcttcttaggaagatattgtctatgtattttagtttggaatcagactggcacaggactcggcatgcactataactcttactgttctattttttcagcttggatttacactgggcaacgtggttggaatgtatctggctcagaactatgatgtaagtggccatatccatgacttccttgattccatgtaactgttttagagtgacttttctttgtttgaggtaaggtgtgacttccagcataaatgtagtccatatggctgaggcaaaactcctgaatattgtagaatggtttgccattgtttgaaaaagtaatccaactcatgaaacagctgtcctccatatcaccacaagagggcaaaatcccttcagtttagctgggctcatgatcatcctcagctgtggcttgttagccgagagtaatattaagttgggctcttaaaattctttgggaaatcatgtgataagtgagaatttaaaaattaattggataatattttcagccccactatccagtagtagagatgacttagaattttggagatgcatctgggtagggggactgaaaaatatagatctatattccatctccaaaattctaagtgtagcacatttggggacagagttttgtgaagttgtattaacctcactttataggtggtgatgttgaaaaccaggtggtgcagcagcctatgaatggggattctggacaggagtttgaaaaagacaagcaagggaaacatgatactaacttgatttgttataaagcttttcatatgacaaagggaattcctttgtcaaccttcgtttgagtcatacagttttctaggcaaaggctacattatgtcacatttattgtttatatgtacaacctttacttggcaccagatcgagcggaagtgggtatacaaaagtagaagtgggtttcagagaaggagcatacttatctgacaacttctgatatctttcacatgcagttataaggctaagaaataccactattggccgggtgcagtggctcacgcctgtaatcctagcactttgggaggccgaggtgggcagattgctcaagctcaggagttcgagaccagcctgggcagtgtgacgaaagcccatctttacaaaaaatacaaaaattagccaggggtggtggcatgtccgtggtcccaactacttgcgggattgtggcgggaaggatcacttgagctctggaggtcaaggctgcagtgagctgtgtttgcaccattgccctccagcctgagtgacagagtgaaaccctatcttaaaaaagaaagcaaaaagaaataaccactatttagcagatctcttttggaggaagataaaagcttgtaaaaccatttatctttggaggaagagaagtacaagaatattgattaggtagatgcaaaattataataaccttttttctcttcaaagataccaaacctggctaaaaaacttgaagaaattaaaaaggacttggatgccaagaagaaaccccctagtgcatgagactgcctccagcactgccttcaggatatactgattctactgctcttgagggcctcgtttactatctgaaccaaaagcttttgttttcgtctccagcctcagcacttctcttctttgctagaccctgtgttttttgctttaaagcaagcaaaatggggccccaatttgagaactacccgacatttccaacatactcacctcttcccataatccctttccaactgcatgggaggttctaagactggaattatggtgctagattagtaaacatgacttttaatgagtagtgtcttctttatcgtttgcgatttttactaccttttttcaaaagaaaaattgatgagttttgtatagctggtcagatacaaataatagtgacttcacagtttagtaattataatgggtacttgttaaacatttggtactaaattatgttgctgcaaagtaattaaaattagtatctagagctagtttctggtgaattattcatttattttgtactgttgttaggcagctctgtagttgctaatttaaccaataagtcaatttgctattcatgaagaaacgattctgagaatcctgtcaggaattggggaatgaaaaaatacacaaaataatggtctttgtcccagtagagttcatagtctatttagtgtgcatgtttttccttaatgatgtatttgatctgacttttttccttctcaaaagaatcatacttgggattacaggtacatttgatgttatatgatggataagtgaaaagtttttaaaggagattttataccttttcacattaaaaaaggtatttatattattactttgtagtgattgtcttaagaaaaaatatagcccaaatgtatagtaaaatcagcagctcaagaagaatttctgcttctctttgtagttgatgctttgttttttcctgcagtcagaaattccttgtatttgtcaaatgtataatcagcttgtattgtttttaaattaaaaaaaaatttgaataattaacttttgccatgggacaagatacaaaagtaatttcatataaagggcctctcccacccctgttctctggctcctggctcctgtttgacaagttactgttaccacttcgccttatacttttgagaaagagtctgtgcctaaacaaacacgtgtaacacaaatagtaactatacatggaggtctagccctcgccttttttttttctttttttcttttttaatggagatcattctataccagcatgtaagtagcaaggaacctcattctttttttggctgcctaaaatttttttgaatagatataacataattgatttaatctgctactggtgaatgcttaggttgttcttttgctattacagtgataacttcaatcctaatgttattaagcatatcgattcagggtatagctataagatgaagtcctaaaagtataatttagactaaatacaaatacccatttcgctagctgttttgtttcagaggacttgttgagcagcttcactaataatgccatttttgaagacatggcaggttcagaatcaataaactggaagaattgttcagagcatcttttttcagacagtgatgacattgattctgtatatgataaagtgattctgcttctctttgacaacttgcatctctcctacatggaagtaagttttattcctgtcaatgttgtctttgtgtgtgacagattaggattaaattatggtttgacttttcctagcagcgtgatcatgggcaagtggcttttttttttttttttttttgagacagagtctcactctgctgcccaggctggagtgcagtggcacagtcttggctcactgcaactcctgcctcccggtccaagtgattctcgtgctgcagcttctcaagtagctggcatcaccaccacacctggctaatttttgtatttttagtaacgacgaggtttcaccatgttggcaaagctggtctcaaattcctggcctcaagtgatctgcccacttcagcctcccaaagtgttgggattacaggcgtgagccactgcgcccagcttttttaaacttttagattcatttaataggtaaattgcatgtcacgggtttgtagcttattctttcagaaactcttgcattatctgtagacgtggacgtaaatatccacctcatagggttttcataaaaaataattgagataatgtatgtaatgtttcacagtgctttgcagactatctaataaatagtagctattagtacaaacttgttgctttttggctagttctgcagtcacttttggaatcagtttatgaaccacacaaaaagtttgtcattattctgtagtcgctgtttttgatactgatgaaaatttacagtatccacctggattgtaccatctggctcacaagatttgatcttgaattgttagcacaatgaactacagcctcaaaggatgaaagtttaccttgcgggtattaaaaatgctagaagcttagaaagctgtttccaaagagggattccttgttaccaaccatgatagcattgctgtggtgtgtgtggtctcccagagtggctgctttgggattggaggtcacttagctattagaaggataggtctctttatattataccttgtaaacaaggctcatttggacttagggcttcctttagaggtgacacttaaccttggcaaggggatcatttctaccagtgggatccatctagcagatcagttcttaaattcggcttcatcatgtaaactgctacacagtgcaggctgactttgcattcaccattcagataccctctcctagttttatgttcttggcagcagtgaaaaggacctcagagttctcaggcaagatgatcttcactgagctatatgttggaggagcccagaaccatcatagttttagggtgtagttgaaaatccttactgtgaatggggtttatggcactgaaagttaccccttccctgggaattttgtaggaacccttgccacacagatcaatggagtggtgggaagaagcaccacaagacaggccttttttttttcctttaaaaaatgtttgaaagctcctactaatatggttaggaaaacttctgccacatctgaccataaaatgagagttgctcaaaatactagtgcagattactctgacatgtgaaatacaagacttcaggattgatgctagaaaaatacagtgaacttgaaaagcttatccctaaactgtaatcctcccaaacaaaacaaaaacaaagcaagaaaaaaagccttatttaaaattgtcaaaagaggctgaattttatgaagttaccaaaagtatatctttagacttttcttctgatttggtattttatgtatttaagcagttacctgattctgatttaaaggagcaattttatttctctttttctaactcatttacccaatgagtttgtgtgactatagagaaaagtaggctgtgtggtttttctagttgtctatacacattgcctcatagagcatatgtacccatggtacacatgtgtaatgttttcatttccactaacccgttcactccctagatactatgctggtgtttttctgctgaggtctcccacatttttaatgccttttctgagttattcctcctgaaggaacgttatatactgtactggagcaaatgaggcttttcttaaacttccagtcttgggttttgcatgtagcatttccattccttacagatgaatgagttcttttttccctattgactattagggcttcgccttgcggaaaggaatagggtggtgaagggaaaatattttgagatcttgcctgctcttaatagggagtttgtgacacttagattttcaagtgttcattttatacctttcccaaatctggttgcagtgtatttagtcaagcattgaagacctaacatatggcatgttttgcaaggcacagggaaagaaaaatgaactgagaacagtgagaccttgtggaagttcccagggtgggacgttgaaaccctgggtcctgggccttcctgaatcacagcctcggtgggaggattttatgaaaggtacagtgcagttgcatcatcttacacagttgtacaggactgggaccaaaaaaggtgccatgtaaatagtgcatgcaattgtgttcagtgttcgttcagtgaggacagccccactgagctacctgtgtactcaggtggtctcggttccctttgcgttctaatggtttgagtttcttgctgtattgagagtgattctagtgtttagagactttttcaaacatcacggtctccagatgtgaagccaactacctcatttattgtatggttgaacaaacagatttgttctgatgttggaaggaaaactagctgcttttgatttgttaaaagatggtacatgagtgtttgtgatatgtaaatactaatgtgaaagattgtataccatgtacttaatgtctgacttagtggaatttcaaaggcaactttgactaagcaaatttgataaagaattggtttaagaataagatgcaactatacttagtgactttggctcacagtttcatcatccataaaatattagaagataaaattaaattatatttgcaagagacacaagactattaaggagggctagaagttatccaacagtggagtgatttagatgacaaggagttattaatcatatttgccatttcctgtcactggaatgggtaaccttgcttggcttgaatctccagttctgggaaagaacagtgctgtgcttgagagcagtttagtagaccagactgaaataaacgctctttctaaattggctagttgaatgcattcactactcggaacacctggcagcagttgctggcacagctttcccttcaggagtgagtcaggttctcacatagttgaatactggccagtaattctaagtccacttaattctaaatcaacttttctttctctttttacttatactcccagaatcttacagattgcacctttgtcctattgtctgacttaggggcaaaggatctgtgttctgtgatggttcataaatgtgatgtttggctgctctgataataagagtaataccctgactcttgaaaatttaaagcaaatgactttagagatgttggctggatcaaatcagctgtagcgtacctaacattggtatttcatttgggcttgtgaggtcttaactattatattggacaaataattttggtctaactcacagggcagaagcactgatgttcccattaaccatggtcagtcacattctgtggttgaagcacatgtttgtggtggtttcttttgctccaaaagaaattttacaactcacagatgcttgatcaattaagggagtgattttatagataaattattgtgggaaaacagcacgtgctatcagtagtaacttaaaatcacttaaaacagtgcagtaattgtaggggactccctttcccctaagatatatttagcaaacaactgatgggagaaataaaaaatatgtaacactaacaaactagagggattgagagctatctccagtcatgtagcaaggatatctatcatggagaaaacggcatggtcttcaggaggagaaagaagccttgatttccagacagttgttttctcatgggacacgtttattgagcatttacagcttaccacattgtagaagttgctgggggatttcaagagccatagacagctcttctccctcaaggggctcatgtagggatgtgtaggaagtgacagatgtgtttggacctttggctactacctaatactttgttttctcaggaacaaactacttattaaatccagaagcaagccagtctaatataataataatgctgtggtcttaattatagttttggtttcccttataagacacataggaaagttttccctccctcccaaaacttaaccaaggcaatagtatttctggttagtgacaggtggagagagagatgttaatctcaatgtatcttctttcttgacttccctttccttttgagacggagcctcgctctgtcgcccaggctggagtgcagtggtgcgatctcagctcactgcaacctctgcctcctgggttcaagcgattcttctgcctcagcctcctgagtagctgggattacaggcgtgcaccaccacgcccggctaatttttgtatttttagtagagtcggggtttcactctactaaaaccctgttggtcaggctggcctcgaactcctgacctcatggtccgcttgcctcagcctcccaaagtgctgggattataggcgtgagccaccgcgcccggccgacttccctttcctttgactaatagaacaacaccagcctctttctgaaagggaacactatatgggaatttggtgccaaggtaaaggggagccattgaggagagatgcttaaaagggaatgaaagtggtgatgaaagagtactaattgccatgaggaagacctactaagaatgccttcttgtggagagaacttaggatagaagcacttaagagaaaggagctgatggccgggcgtggtggctcacacctgtaatcccagcactttgggaggctgaggcaggtggatcacctgaggtcaggagttggagaccagcctggccaacatggtgaaaccccatctctactaaaaatacagaaaaccagctgggcgttgtggcacatgcctgtagtcccagctactcaggaggttgagacagtagaatcgcttgaacctgggagtggaggttgcagtgagctgagatcgtgtcactgcactccagcctgggcaacagaacgagactccatctcaaaaaaaaaaaaaaaaaaaaagagagagagagaaaggagctgacattaggtttcgaggtctgggtgaggtttcccacaaaaaagatgtcactggtttcactctgccttggtaggatgacttgggtatctttctactcctaagaaggtattaagaataacataaaacctagtgagtttgcaaggcttagcctccgaagggtgactaagacaggctttcttttcctgtcttcagaatgaatggattacaaagactagatacaactttaggttttctttgtcttctttttttttaatttaatttttattttctttttctttttctgtgagcctatggcttgtattgaaactttaggttttcttgagctgtggtgctgagggcaggaagaagacactaacagcgaagcatgtgtttgtggttgttggaggattcctgcagttcacttgaggtggcggaatcttctggtggagggatgccctccggcgtgggtctggggttgtgtgctcctggtggtcctagtggttttctttgcctgtggtccagatactgctggatactggcagctcctgtggttgggccagtttgtacacttggcgttaggcttgatcagtgatgttgctgaccctcgcccatgctgggtaagtgtccaggtggaagaggctaactccccaggtgttgatggccaccattactatcaccagtccaataacgttgactcccaggccagctttcacctgcaggacacaaaccagcacaccctagtcactctgaccagcagcagcacagatccccaagtcccccttctgttcctgcagcctatgccttgtggcccagattagttcccagttgctgcctggggtgctctagcattgccttgtagggtgcacaggactgtggaaaggcgggaaagggacaattagagtcatctccacttcatgcttacagcactctcactagatttcaagtggaagcctagaaggtatgttagttacttggaacacccactctgtggaacgtgaagaggtatttctgcagctcactgatagggaaatgtgctaaacaaaactaaccagatctctgtaaagcaggaacttctcagagccccgatgcgttaatacagggaacttgtacgaggggctatattacatgaagcctttgctactgttacctttttttaaggaacagctcatggaaatagtgtaccttgagttcacttggggaaacggtttagctgcctctgctcagctcttcctcaagaaggggacacaggcatgctagctggaatggagcctttgcttctttcctctgagggaattacacctcagtgactgacccttttcacttctgccttgacattgacacccacaggctgaagtgggggatgtgagagatggtgggctggcagagccagatggaaaaatgtgtcctgtaaggatagctggcatcttcccaaggagtaaccaggcatatagcatgtagccaagacaagtcactagctggcattaagttgggaaaattgcaaggggaaagtggatgctgggaaattgtttcaagcaaatggctagacgtgtcctctctctcctgccgtctggtcggggccccctgggcattttggcatggaggacagcccatgtttccccacaggtgagagggcaggttctagcagggctaaattaaggagtgttagagttgaaagggaccccatggagcatccagctggaacctcatcatttagcagatgaggaaattgaagtcaaggtagtgcccaagctgggaatagtgcccaagactggctcacttcttgtcacctggtacctttgaaaattactgcctctgatcatttgattccagtgtagaaagctaggggtcagtggtgggggcacacagcagaggaagcgggagacaggagagctggggtagagctcagcgtgagcagactgagggttacgcagtaagatgggaggggagagggatggctgggacactccattcaaggaagtatcacggatgacttagatgagcagttcgaacagtcgaaaggacctgaataggctgagagcagtgactcacacctgtaatgccagcactttgggaggccaaggcaggcagatcatctgaagccagtagttcaagaccagcctgaccaacatggtgaaaccccatctctactaaaaatacaaaaattagctgggcatggaggcgcacacctgtagtccaaggtactctggaggttgaggcatgagaatcacttgaacctgggaggcagaggttgcagtgagccgagatcgcaccactgaactccagcctgggtgagagtgagattctgtctcaaaaaaaaaaaaaaaaaaaaaaaaaaaaggataaaaaaggacctgaatatctgccaccagcctgaaactttttcttcctttcctctatttgttactttacagggctgcccgagaacaaatttggctcccatgaaaaaaaattgtcagaccatcagtaattcgtatcagtgttttccatcctctccccccccgctcagccctggatactaagtatagcagcaggtgtataggtagaggagtagaaggtagataggcccagtgctaaagtcagcagcaaaatatctcaaagccatcttttgtgagcccagctgaaaatgaaaatgaatggtggacgcttcattttccattgttgcttcaggggtttcttgcctgggccggggtggagggtgagttgagcagaagaatcctgttttctcaaaatacctttcaccctaggttcatacacttcctcattctcatcttctgtgaataaaccagtagctttctcaatgaatgtctccactggcctaatgaagaaacgaaggcaaaacttgtttgcacaggctcatgccttctggaactgggccacagcctgtctgttccagctttctctacctaggtacccacttccttccagggaagggttggggagagcacattcttgctatccttggtgcttatagaccaacaaaaacctttttataggtgctattcttgctaccacactgggtttagcattgacaggccagggacactgctacaaaataagctttgaaagaaggctgcagagctgtcatccctgtcctcatggcagctgggcctggcagggagagggcaccccaactcaccatatctttgatctggcagtgcccatagctgaagacgatggcattagggggattgcccacaggcagcatcactgcaaaggagatgcacatggtgactgggatcagggtgtagagggggttaatgtgcagcgtttcagactagaagagagaattcacagaaacattcacgagattaggcttgcatttcctgtgcttaatgtcaggagcatgctttaatgatgacttggtgctggctaaccttagcaggacactatgtggaggggtagttaggtctctgccctctggggcatgcagtcccttgacaacctgaataggctgatgctgcttacttgctccacatggaaatatggctttgagttactccctagaagggatgtttgatttttttggtgttattctaaggaattggttcctaaactttattctttagctgagacttcctttccttaaactcaaatctcatgaataggttgctttgattgaagtggggattcctcagtcttagggtacagaactaaggtggttttttttttttttttttcctagggaagataattgttttatatgggatttgaatccatggtcctgctctcaatgttgccaagttttaactgcactctgttgctcgcaggagcaagatgacggaagggagaagttggggtcatctgagcaatgagcagagagtatggctctggggttagaaagagcagtaggagactatgttgctggggtcaggaggggccttcatttcctggattctgcctcccagaatgggacagatgaagcattgggtcagatgcatcacctttccggctgcttggccaggctaagatctggccgttgctcccctcccttattctattcacaacaaattggtctgaggttgtggagcaaagctgaccttgctcccaatcctcagaacttcccggaggacagtgaaactcagtggaggctgtgatgattgtctgagttgtgagctcagtcttttcagctgtgtgtgttttacctccattttctgtggctacctctttggccctactcaggttttatctcaggagagaaaggttgataatgattgcggggaattgaaatgatagggtttgaatattattttgtatgttctttggtttccactcagggctgcccctaaactaatcctactcttttggacaaaggtttctagaactcttgctatgatttattagtgtatctttttatttactagttgctatttgaaaaacttcatttttcaaaataggaattgaacaccaattagtgaaggctgtgggcagtaaccagaggagagcctacagctacggaagctgccctgagccaggcctgccactccaggcccctgggacagcctgtctggatgtctgggagctccgcattactcaccaggctgcacaggatgggcaggaagatggtgatggttgctgggttgctcacaaactcagtgacaatggacacgaggatgcatgccagcagggtgacagcccacggtgggaggctgctcagggacaacatctggttcccaatccatgtagagaggccagagctctgtaggaagaggtgttacagtaagaagaaaggagaagaagcacatcccagctagagatcttagacttcagaaggtcagggatgttggaaggctggacgaccagggatcctctgatctttagtctgactggaaccagaggtgttcctgatttttatggttcgatcatttttgtagttcagaaaattggtttttatgattgcacaacattcagtgtttgggtttcgtcatgggacatggagagccaccctaattgccatgatccttcttaggactttgccatgacaacttgtcagaggttggagatctttgctcctcaacttcagtctcgttaccctttctctagcctctgacatccttaccctgtgcattgattcgacaatgcccttcacagagtgccaagtatagccatttaatctgcttttgaaaccagtttattttgttacctaaaaaagggaaaaatttatcaacatctgtggatagtagcctttcctgtgtttttaaggagatgaacaggcagggctgagcactggaggttttacaggagtaataaagtagagggggataaaagtctatttccacttagtggagaaggtgcctcttaagtgccatagaggctggtcattcagccccaacctgttttaggttcaaatctcatcatggctgacattagttctactatctgtcaggaatttttttttaattaagaggagggtcttgctctattgcccagggtccaggatgagtgcagcagcatgatcacagctcactgcagtctcaacctcctgggttcaagcaatcctcccacctcagccttccaagtagctgggaccacaggcacataccatcacgccctgctctttttatttgtagagacagagtctttccatgttgcccaggcctgtctcaaacagacctgggcttaagcgattctcccacgttggtctcccaaagtgctgggattataggtgtgagccaccgcacctggcctgccagtgctttttgaagacacagatatgaaggcatattgaccgtcctgaatttattccactagcaacagttacacacatccacttaaataaatgaacattctggctgggtgaggtagctcacacctgtaataccagcactttaggaggctgaggaaggtaggtcacttgaagtcaggagtttgagaccagcctggccaatgtggtgaaagcccacctttactaaaaatacaaaaattagctaggtgtggtggcacgcgcctgtaatcccagctattcaggaggcagaggcaggagaattgcttgaacccgggaggcagaggttgcagtgaaccgagattgtgccattgcactccagcctgggtgacagagtgagactctgtctcaaaaaaataaaaaataaatgaataaatattcccacttggaatgattcatttggttggaagatgttcacctgcaaatagggagggctacctccctctcctaaaaaatgaaaggtatcattgagttttgtattaaaaactccaggttgccatatgcatcctgcctcccttcctctctctcttctatggtgacaggctctgccttttcccatggtaccctagaaactgcgaatgcaatgacaatatggagagcagggacagaaaggaaggtgagacatttatgattcctcaagctcttaagtcctgctacacatgttttactattaaataccgccttccctacactgccagcatgttactttttctcctctcttccaaattcatcaaaagctcatttcatctctacagtaccctggccacctcctggcaagctcatagacaactgaggaccaaaggccttcagaattctctctgttgtagccacttcaccttgtagaacttttcttcctttttttcctctcaactcactgagagcttacaaacccaatatgtgcagactgcatcaattaatgcttgtttcagagccccaaggcctccaggaactctttaattgttccctctggtaacacattaaaccttgctcattggacagaggcaggggaggaacagcagttagttgaatggaaatggtacattatataaagagatggagttttacttaacgttatccaaatagtactggtaagtaaagtgaaataattttttttttctttttgagacagagttgtgctctgtcacccaagctggagtgcagtggcaccatctcagctcactgcaacctctgcctcccgggtccaaatgattctcatgcctcagcctcttgggtagctgggattacaggcatgtgccaccatccccggctaaatttttttttttttttgagaggaagtctcactctgtcgcccaggctggagtgcagtggtgcgatctcggctcaccacaacctctgcctcccaggttcaagcaattctctggcctcagcctcccaagtagctgggattacaggcgcgtgccaccacacccggctaatttttgtatttttagtagagacgaggtttcaccatgttggccaggctggtctcaaactcctgatctcaggtgatccacccaccttggcctccccaagtgctgggattacaggcatgatccactgtgcccagtccctggagttttgcttttgtcacccaggctggaatgcagtggcacgatctcggctcactgcaacctcctcctcctgggttcaagtgattctcctgcctcagcctcccaagtagctgggattaaaagtgtccgccaccacgcctaactaatttttgtatttttagtagagacaacgtttcaccatgttggccaggctagcctcaaactcctggcctcaagtgatcctcctgcctcaccctccttaagtgctgggattacaggcgtgagtcaccatgcccggcctagtaaagtgaaataatttattggcaattatttttccagcagaactattttatactatgatttgaaataaattagacttcaaagttagacaaaaacacaatcaaaggaagtcttaattctttaaactcttggtatgaaaacattttacaatggattttcttcactaatgtcaaaactgaaattatttcaaactctgtaggaatcacatatatgctacctaaattattattaaaattagtagaggcagtaggctttacagaaataactgtactatctctaatatgtgcacaattttgtttttattgttttttttttgtttttgttcttgttttttgttttggtggcaggggatagggtctccctctgtctcccaggctggagtgcaatggtgggatcttggctcactgcagctttgaactcctgggctcaagcgatcctctcacttcaacctcctgagtagctgggactacaggtacgcaccaccacactgggctaacttttgtttttgtagagataggatcttactatgttgcccaggctagtctcaaactcctggactcaagcaatcctcccgccatggcctcccaaagtgctgggattacagacatgagccactgtgcccagcccaattttggttaaaaaaaaaaataaaatcttgccaggccaatcttgtcagaggccaaaactaacaactgaaattcaataaagataaatataaatatcccgaacttggaatgagaaatattgatggcatatatataggatggggtgaaggtccagtaatgatgaacagttaaaaagaatgaagggttctaattgactgcagttttaatataattgttgacatgagtgccaaggatgctttataaaagcttccataaaaatgcagggtgctgcttgaatcagaggtgagagatggggcttgggtaatataactactctacttggcagtagactgaccaccccatagactatgtttgaataaggccaagtgtagtgagtcctgaggagagagaccagatggtatgggtcactactacttgattttttcatgttaataattattatctaggagagggtcccagagaacagagctagagccaacagtggacagacaacatgcagaagacctgagctgaaaataaggaacaattattctttgttccctgcccagggagtaccttgtcactggaagtgtccaagccagaagtgccatggaggggaatctcaccatgggcaggagataggactcattgttttctaaggttctccttagtggagtattctgtgaccttattattaataatgcagatagtattttgggccatgaaaagtgaaccaagccgggcacagtggctcatgcctgtaatcccagtactttgggaggccaaggtgggtggatcacttgaggccaggagttcaagaccagcctggccaacatggtgaaactccatctctactaaaaatacaaaaattaagctgggtgtggtggcgtgtgcttgtgatcccagctatttgggaggctgaggcatgagaattgcttgaacctgggaggcaggggttgcagtgagcagagatggcgccactgcactccagcctaggtgacagagtgagactctgtctcaaaaaaaaaaaaaaaaaaaaaaaaaaagagaaccaagtttagcatgggataatgcatgtagatgagatggagggagaaatagtctgaaccttaacctaattcattcttaagattgttttctattttatggagggctaacactttaggaaattcaatcatcccctcacatccccaccccacttaaaagatctcatccaaaatcctcccagtcagaaaatcaagaagcatttacatcagctttagcctagattttccaaaggggctcagtctatgtattgtggacagacagttcaccatgtgtcacttggagcatctctggtcccttacagagatcgcctattaccactcctattacctattacctatgtctatctcctgttaccatctcctattgccactgtcaaatcccagaaggagatggctgcaaactccaataaaccttttgcttgtagcagcatgggccccatagatgaactatcaatgcccatgtgtcatgactgtgtggcacagcccatctgttcctctgttcctagtagcgctgagaagcttagggcagcagaggacatccatcctgctctattgtttcccttcccagggactctgtgaacattctcagctgctttgagtctttcctcccatgcccataatgactcattaggtaaagatgctggaaaagtatttgacccataagaacagaattcaagccgggtgcagtggctcatgcctgtcatcccagcattttgggaggccgaggcaggtggatcacttgaggtcaggagtttgagactagcctggccaacatggtgaaaccgcgtctctactaaaaatacaaaaactagctggcatcatggtgcacgcctgtaatcccagctacttgggaggctgaggcaagagaatcgcttgaacccaggaggcggaggttgcagtgagctgagattgtgccactgcactccagcctgggtgacagagcaagactgtctcaaaaaaaaaaaccccaaaaaaacagaattcaagaattaccttgctaccagaagccagagcatagcctcccccaaccagaatgacaatctcccagggcatggtcttctggaagtccttccacgtgatgatgggctcggtccccagtgagtgctcctggttctctcctggattagagagagatgtaaagccagataaaccctgatggacgtgatttgtggagacaggagcctaattggtgaagtggatgatttgggatactgctatttggggcgggggccgggaggggggtgggaatctgaaggaccttggtaaatctctttgggagtattagtctgatttgggtattcttcaactagagctacccccagtttcttcccttctctcaccatcattctttttcccaaagcagggcttcttcgctggaatgaggaagaggaggaagccaaggaagacagagactgtggcatcagtacggtagcctttcctagtaaagagacaagcatagctgaggagaagggtcaggaattttcaggagtaacatgatgtctcggagcagtctgtccgaacacagtgcctctttttaggacttatctagaggtaacattaagcttagttcttgccagttgctgccacgctatccactttggatttaatgggcaatggcgaatgtctggaggcagggcctggctagagtagatgtcttaatgtgaatttgtgagcccgttaattggggcaagtcctagatggagtgatcaggagtggagttggttagataaaggagaatagaagaggatgagacctctttcctatgaaggccacacctccatgtgcctcccttctcaagtgagaagttgatttcttctttacagttggtactttatagatgtttcctcctttaactttcataaccacctatgaggtaaccaagcttccaagagcttatgtaatgagactacatgcagcactggattccaaccccagaccgggactccaaagagtgtttttccttcctgatttcacactgcagcctccctaaaaagagtggatttcctggggaatgaaatcttcctggcttcatatctgcccctgagagtctatgaagtcttgcctgaagaattggggtgagggggttgttcttaaggaggaaatgatatcacttacttttcaaagaaagaatcccagccagggacaaagccaggctcccgggtaaaccacagtacggtcatcaggatgaagaaaaatccagtcaccatttctgggtagctataaaataaaacagaaagacccactcaggaactgagaaatttctcctagctttttcactttgcttctggaagcttctgtatttagagtttcaataccctagacataaaacacactcactgcctaaccactatgaatggctgacagtgagatgaggagaagactgagtggccccagaaaacatgacattaaccatgttcctggagaaacttcagtagaaagggcaagagaacacagcactgtacatttgtaaaataatttcaaatacagcctacaaggagagtgcagtgaaataggcagccccaaacactgtgctggtgggaggaggaattgctacaacctttttgaggccatttgatgatacacattagaagccttaaagttggtcattccctggctgggtgtggtggcccacgcccgtaatcccagcactttgggaggccgaggtgggcggatcatgaggtcaggagatggagaccatcctggctaacacagtgaaaccccatctctactaaaaatgcaaaaaattagctgggcgtggtgtcacgtgcctgtagtcccagttactcgggaggctgaggcaggagaatcgcttgaacccaggaggtggaggtttcagcgagccaagattgtgccactgcactccagactgggcaaaagggtgagactccatcccaaaaaaaaaaaaaaaaaaaaaaaaatagttggtcattcccatcatctaaccagaaatgcatactgagacattaattacctattaaaaaatggaagcatcctctatatccaatggtatttcatgacactgaatacattttgatacaactctgtgttaaaatgagatgcatccattgataatcccatttccacagcagagttaaggatgtggaacatactgtactgagagtacaaagttgagttgagaaaggcaaggtatacaacagtttcagtgtggactgaattttaccaaaaatacataagagttttgcacaaaaaagcatggaaagaagtgttaacatggggtgctaagaatccagtcaattttgattttttttcttatactgtcttgtatttttgaaatgttctacaatgaacatgtattccttttataacttaaagctttaaataagacagtgtctacctggattgttttatgcaaagttgtatgtgccaattgccataaaaggagaaatagcactaaataggaagctgggtccctgtggaaaagctgatggaagtggctgagggttggttagtgatttcttggacgctctctctccaagtctgtctttgatactgccttataccctaaatatagcacagagtctcccatctccctgggagtgataaccgcaccactgtggtggcgcgtctgagcaatgagaggtggcctggctttggaaggaattcacattttccaagagggtggctgtgctgtctttcccttcttctgcctctgtctgctccatggtcagcactccaccccatgccatggctataccctggactctgaatagagcaaaggggaagggtggccacctgaaatctgggccagcttggctttg

SEQ ID NO: 68 - DNA, 5451 bp, Homo sapiens
tgggggagggggaggccttttttcttagacaaaattgggcattcccaacttacacgcatttagtgcataagctgacatttcaatgagattgtgaaatgacaatgggaagggccctgaaaaaaacatcaagcataaaaggcctgggaggccgggcactgcggctcaggcctgtgaaatcccagcactttgggaggatggggtttcgtctctactaaaaataccaaaatgagttggttgtgatggtaggtgcctatagtcctagctacaagaggctgaagagaatcgcttgaacccgggaggtagaggttgcagtgagcagcggttgagatcatgccattgcactccagcctgggcaacaggattcagtctcaaaaaaaggaaaattcagagagtaactgaagttttatcattaatgggactaaagtgctttagcccagaaacagatttgtaaaaccattactgctaacaaagacatcattataagcacagctgtactgacactgaaattaaaatttgacccatcttggaattttggccccttggtcctgtttccctgggacagattgccttaaaccagttgtgccctttatacccacttttggcatccctaaacacttagtgcttgccaaaaacctatcaccatggctatggattgtagattttcccaattccattaatgcctatcactagttgggcatttctatagtattttctataacttattttcccagcctcagactcaacatgactttctccaagtagctccatcaatactttaagctcaaaaccaattagcacaggggcctatgaaacctgacaatttgctcatatggagaattaggaaatattgcactttaatgcataagacaccttgtaaaatacccataaaattttaactggctgcagtgttaatgaagcaaataccatggagttataatagcttttgaatgtgatttatgcttacagctaatgaacagatgaaaagcactgtccttttttaagtaaacactacacactccagatggattgcaaaaatttattaaaattggagacactgttttaatcttcttgtgccatgagactccatcaggcagtctacaaagaccactgggaggctgaggatcacttgagcccagaagtttgaggctgtagtaagcttcaaaggccactgcactctagcttgggtgaggcaagaccctgtctcaaaaaataagacaagacttttagccacatttacagacttcaattaaaatgctacatgggaatgcagaatatcagatattttattgtcatcaagtaatcagtgagagagttcaagttggattgagatcatcatgtattctgcaattataatagtcccaacatttccaatcctcacctttcaagcagtaagctgcatgcttgcttgttgtggtcattaaaaaccctgaaagcgaagccaacatattaatcagggcaaaatacaaatgtgtgatgcttgttagtagagtaacctcagaatcaaaatggaacggttttacagtgatatcattatatttcatttggcagaatcattacatcattggttacactgaaaatcatcacatgtaccaaaagctgactcacctagtttaggataacaggtctgcctgtttgaagatgaaaaataatacccatttaaaatttgccctactcaatttccttctcagtcacattttaacttttaaacagctaatcactcccatctacagattaaggtgtatatgccaccaaaaccttttgccaccttaaaaatttccttcaaagtttaaactaacctgcatttcttcaatcatgaattctgagtcctgaaattaagggacaatacacagtaagacaacttgcatacaattaggagtaatccaaaatctccaaacattatagaatggctatattccccttttcggggcataaactagaatgttaccgccagatacatcagataggagcgaaagacttaatattgctcatcagcgttagtctgctgaactatgttatcatcattgtatcggctttacaacacttaaaaaccagacttgaggcggggcatggtggctcacgcctgtaatcccagcactttgggaggtcagtgcgggcagtatcacttgaggtcaagagttcgagaccagcctggacaacatagctgtacaaaattagccaggcatggtgtcacatgcctgtaatcccagctactcaagaggctgaggcaggagaattccttgaacctgggaggcagaggttgcagtgaactgatagtgccgctgcactccattctgggcaagagctagactcttgtctaaaaaaaaaaaaaaaatacttggaactgctatacctttgcttctttaaaacttgctccacacagtgtagtcaagccgactctccatacctgtagaaatttaatctcctttaccaataggtagtaataggtggatttctaaaagatttcccaactactgtttcagtttaagattaatctccattttctttctcagagagattcccatctgctttaattttaaaatcatcactaacagtagctcaatcaattaacgttaacatcaataaaacatgtacctttaaaaggtatgacaggaactgtcttcatgtccttacccaagcaagtcatccatggataaaaacgttaccaggagctggaatacaaatgaggacttccattaaaatgctaaacatcattaaactcttttcaaagttgtctgtcacagggttcttcagtgttacctttgtctacatgctcatttcaggtcagacatttgatcaacatcattacacaaaccccgttccaaacatgctcaaggaaaacacataaacactgttccagacatgctcaaggaaaatacagtcagtattcatcagccaagccttaagccattagctcagcagtaagcatatcaccatcagcattttcattaagtctatctactgttttcattaatacttaccagaaccattaagctggtccaggcaagttggactccacctaagaaataagagtggtcttagaatagccacagatgagtgttcaccccctgccaaaactaacaaatgcctttagtcagttagagctaattaagaccttcatgttcagtcagcatttgcttatcatcatccaggctgcatttacaaactttcttattaatcataacaagacaagaatccgccatttatttaatgcattcagcactaggaggtaactaaaaagccatttgtgccgtaggaagtttgcccagttacctgcttacttggatacagtttcacttaccatttcaacttccagctttctgtctaatgcctgtaattttaatacaaaattgtcagcaaaaaatattttaatggtagattggtggtacactgcttaaccattaacagcaaaccacaactaccccagatacatcagacagatagtacatctcttcatgattaaatctgctgaactatgcaaccatcatagtatctgttttcacattttcttcagtattaaacttagtatcaatatgagagcaaaattctcatttgaaaagaggggagagaagcactaacatagataatcatcattgctttggctattttctaatcccttaaaagtgagaagtacaaaatagaggtgtctcacctgtgtgccaatggcttgagttaggcttgctctttaggacctgggaagaaacaacttcatgtgaacttaggtgtactctctatgttccccaactcaccattttgggtgcctcagttaagataatggtggttaagatcctcatcattctagcactcaagagtagcaaataaactgtcatcattgtggcacatccaatggctttaaacctttttgagagggaattttcaacttacttcagtagctattctcatccttccttggggacacaactagaaaacaaaaagatgcaagcaaaacagtgagaatgaacctcacagcagtcacgttaaatataaatgctgttaacattaaagcctcagaatagaatttcagaaatcccttctgtccactactcttaaagcatcacaggctgagaccacctcttagatttcattcttcccaccatactttccccaattcttacctgtccataaggtgctatccagagccacactgcatctgcacccagcaccatacctaaagagatcaggtacagattttaattcattgttccacggttcttttttttaaagagtgttctttaaaaaaacggttcgtctttttaaaccaatgatgcaggtaacataccattcattatctagcgacaggtaaaggatacacttaatgttacaaagaagcactgcacccgcagttaagaaccaccgatggcggccgggcgcggtggctcacggcttgtaatcccagtatgttgggggaccacggctggtggatcacctgaggtcaggagttcaagaccagtctcgccaacatggtgaaaccccgtctctactaaaaatataaaagcctagcatggtggcgcgcgcctgtagtcccagttacttgggaggctgaggcagaactgcttaactcgggacgcggaggttgccattaaccgatgtcgagccactgcattccagcctgggtgacagaacgaggctcggtctcaaaaaagaaaaaagaaacactaatggaaatattggtgacactgcggaatgcaggcacttaagcacgtggactaccaccgacagcctttcatgagagcggacggctgatggaggctcgggtcacggcccttaacaatagcttactcgggtgactcggcatgtgccaccatcagagcggttggcattcatcattactctcagatgtccctaccaacacaggcttcatcagaggcagggcacgagagccgaggcttctccgcagttctactctaacatagtcgcagcttaggtcacgcatgcaccatatgtgcaatcccccaccatcctcaacttgtaggccctgcaaaggcgcgcgactggcttagaagtcccagtcaattcagggtgaccttagcagacaaaggctggagcgccccaaaacccgcaacattcgcaaagataaaacatacctcacaggagtcgactcctacctcgaaaagacagtatggtgcctgggctcctgtatataagagttcttttgatgacgtcacgacgcacgctcccggcagcgtccgcttcggctcctccccctaacctccctcaatcttcctctcatccagtatcaccttcttcccaccccttaagtccgccccttttccccgcccttcctcgccattgtgggcagagacatgaccgtccacgtgatgccgtgctcccgccctcctcccgactgaggaggaagagcagagaggggagggggcgcgaggcaaggaaagctctggggatgggggaagggggggcggtgtaaggctgagtgatttccttccggcacgtcgcccg